(19) United States
(12) Patent Application Publication
(10) Pub. No.: US 2021/0261569 A1
(43) Pub. Date: Aug. 26, 2021

(54) FUSED HETEROCYCLIC DERIVATIVES HAVING SELECTIVE BACE1 INHIBITORY ACTIVITY

(71) Applicant: Shionogi & Co., Ltd., Osaka-shi, Osaka (JP)

(72) Inventors: Syuhei YOSHIDA, Osaka (JP); Genta TADANO, Osaka (JP); Toru YAMADA, Osaka (JP); Takuya OGUMA, Osaka (JP); Ken-ichi KUSAKABE, Osaka (JP); Frederik ROMBOUTS, Beerse (BE); Antonio MISALE, Toledo (ES)

(21) Appl. No.: 17/256,742

(22) PCT Filed: Jul. 4, 2019
(86) PCT No.: PCT/JP2019/026598

§ 371 Date: Dec. 29, 2020

(30) Foreign Application Priority Data
Jul. 6, 2018 (JP) 2018-128673

(51) Int. Cl. C07D 498/04; A61P 25/28

(52) U.S. Cl. CPC C07D 498/04; A61P 25/28

(57) ABSTRACT

The present invention provides a compound which has an effect of inhibiting amyloid β production, especially an effect of inhibiting BACE1, and which is useful as a therapeutic or prophylactic agent for diseases induced by production, secretion and/or deposition of amyloid β proteins.

A compound of Formula (I) wherein Ring A is THP optionally substituted with R³ s or the like;

R² is C1-C3 alkyl optionally substituted with halogen;
R³ is each independently C1-C8 alkyl optionally substituted with one or more group(s) selected from halogen, cyano, C1-C3 alkyloxy, C1-C3 haloalkyloxy, 3- to 6-membered non-aromatic carbocyclyl optionally substituted with halogen, and 3- to 6-membered non-aromatic heterocyclyl optionally substituted with halogen, or the like;
t is integer from 0 to 3;
A₁ is CR⁶ or N;
A₂ is CR¹⁰ or N;
R⁵ is a hydrogen atom or halogen;
R⁶ is a hydrogen atom or the like;
R⁸ is a hydrogen or the like;
R¹⁰ is a hydrogen atom or the like;
Ring B is pyrazine substituted with cyano or the like;
or its pharmaceutically acceptable salt.

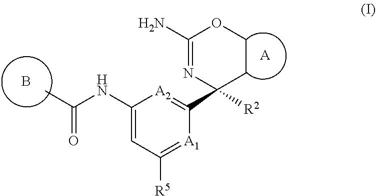

### TECHNICAL FIELD

The present invention relates to a compound which has amyloid β production inhibitory activity, and is useful as an agent for treating or preventing disease induced by production, secretion and/or deposition of amyloid β proteins.

### BACKGROUND ART

In the brain of Alzheimer's patient, the peptide composed of about 40 amino acids residue as is called amyloid β protein, that accumulates to form insoluble specks (senile specks) outside nerve cells is widely observed. It is concerned that these senile specks kill nerve cells to cause Alzheimer's disease, so the therapeutic agents for Alzheimer's disease, such as decomposition agents of amyloid β protein and amyloid vaccine, are under investigation.

Secretase is an enzyme which cleaves a protein called amyloid β precursor protein (APP) in cell and produces amyloid β protein. The enzyme which controls the production of N terminus of amyloid β protein is called as β-secretase (beta-site APP-cleaving enzyme 1, BACE1). It is thought that inhibition of this enzyme leads to reduction of producing amyloid β protein and that the therapeutic or prophylactic agent for Alzheimer's disease will be created due to the inhibition.

Patent Documents 1 to 10 disclose compounds having a structure similar to those of the compounds of the present invention. Each of these documents discloses each compound is useful as therapeutic agent for Alzheimer's disease, Alzheimer's relating symptoms, diabetes or the like, but each of substantially disclosed compound has a structure different from the compounds of the present invention.

### CITATION LIST

### Patent Literature

[PTL 1] JP2017/071603

[PTL 2] WO2015/156421

[PTL 3] JP2014/101354

[PTL 4] WO2014/065434

[PTL 5] WO2014/001228

[PTL 6] WO2013/041499

[PTL 7] US2013/0072478

[PTL 8] JP2012/250933

[PTL 9] WO2012/107371

[PTL 10] WO2011/071135

### SUMMARY OF INVENTION

### Technical Problem

The present invention provides compounds which have reducing effects to produce amyloid β protein, especially selective BACE1 inhibitory activity, and are useful as an agent for treating disease induced by production, secretion and/or deposition of amyloid β protein.

### Advantageous Effects of Invention

The compound of the present invention has selective BACE1 inhibitory activity and is useful as an agent for treating and/or preventing disease induced by production, secretion or deposition of amyloid β□proteins such as Alzheimer dementia.

### Solution to Problem

The present invention, for example, provides the inventions described in the following items.

(1) A compound of Formula (I):

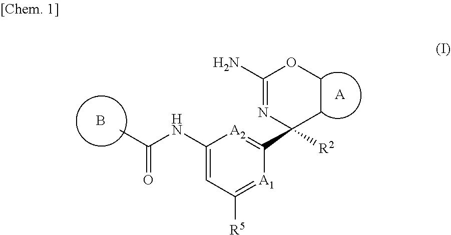

wherein

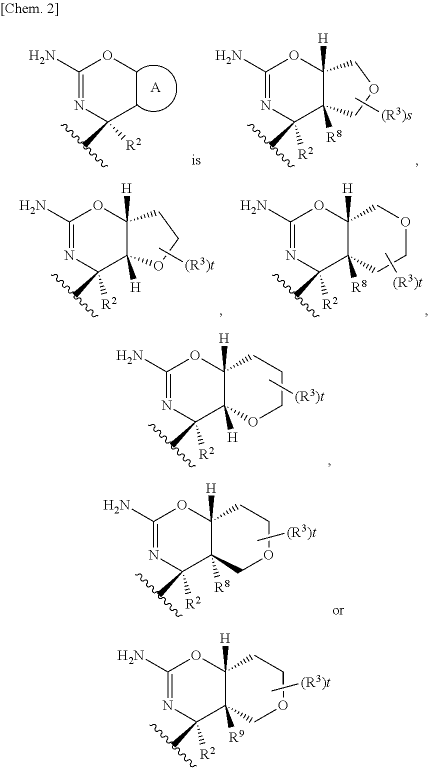

wherein
R² is C1-C3 alkyl optionally substituted with halogen;
R³ is each independently C1-C8 alkyl optionally substituted with one or more group(s) selected from halogen, cyano, C1-C3 alkyloxy, C1-C3 haloalkyloxy, 3- to 6-membered non-aromatic carbocyclyl optionally substituted with halogen, and 3- to 6-membered non-aromatic heterocyclyl optionally substituted with halogen;
aromatic heterocyclyl optionally substituted with one or more group(s) selected from C1-C3 alkyl and C1-C3 haloalkyl; or halogen;
s is an integer from 0 to 3;
t is an integer from 0 to 3;
R⁵ is a hydrogen atom or halogen;
A₁ is CR⁶ or N;
A₂ is CR¹⁰ or N;
R⁶ is a hydrogen atom, halogen, or C1-C3 alkyl optionally substituted with halogen;
R¹⁰ is a hydrogen atom, halogen, or C1-C3 alkyl optionally substituted with halogen;

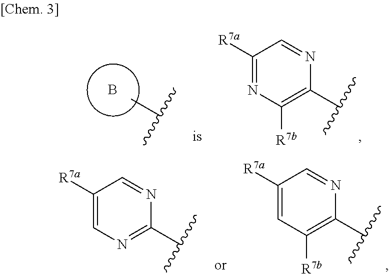

wherein R^{7a} is halogen; cyano; C1-C6 alkyloxy optionally substituted with one or more group(s) selected from cyano, halogen, hydroxy, non-aromatic carbocyclyl and aromatic heterocyclyl; C1-C6 alkyl optionally substituted with one or more group(s) selected from cyano, halogen, and hydroxy; non-aromatic carbocyclyl optionally substituted with one or more group(s) selected from cyano and halogen; non-aromatic heterocyclyl optionally substituted with one or more group(s) selected from cyano and aromatic heterocyclyl; C1-C6 alkenyloxy optionally substituted with one or more group(s) selected from cyano, halogen, and hydroxy; C1-C6 alkynyloxy optionally substituted with one or more group(s) selected from cyano, halogen, and hydroxy; or aromatic heterocyclyl optionally substituted with one or more group(s) selected from C1-C6 alkyl;
R^{7b} is a hydrogen atom, halogen, C1-C3 alky optionally substituted with halogen, or amino;
R⁸ is a hydrogen atom or halogen; and
R⁹ is halogen;
provided that when R⁸ is a hydrogen atom, then s is an integer from 1 to 3;
provided that the following compounds are excluded:

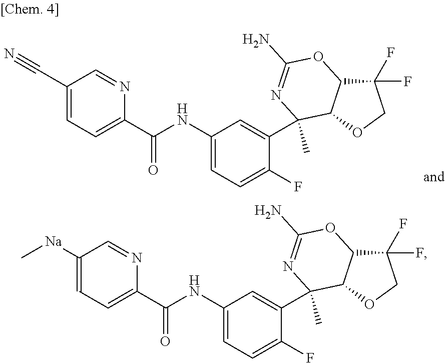

or its pharmaceutically acceptable salt.
(1)′ A compound of Formula (I′):

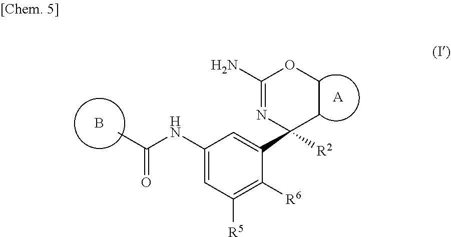

wherein

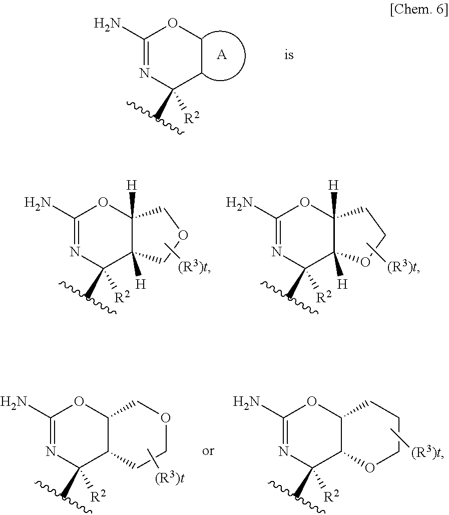

wherein
R² is C1-C3 alkyl optionally substituted with halogen;
R³ is each independently C1-C6 alkyl optionally substituted with one or more substituents selected from the group consisting of halogen, cyano, alkyloxy and haloalkyloxy; or heteroaryl optionally substituted with C1-C3 alkyl;
t is an integer from 0 to 3;
R⁵ is a hydrogen atom or halogen;
R⁶ is a hydrogen atom, halogen, or C1-C3 alkyl optionally substituted with halogen;

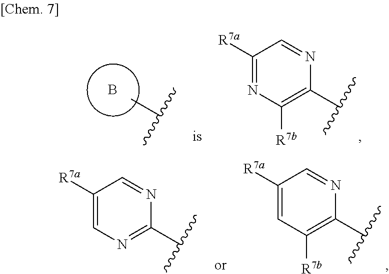

wherein R^{7a} is cyano; C1-C6 alkyloxy optionally substituted with cyano or halogen; or aromatic heterocyclyl; and
R^{7b} is a hydrogen atom, halogen, C1-C3 alky optionally substituted with halogen, or amino;
provided that the following compounds are excluded:

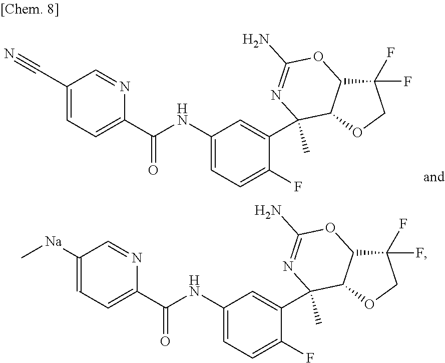

or its pharmaceutically acceptable salt.
(2) The compound according to the item (1) or (1)′, wherein s is an integer from 1 to 3; and R³ is each independently C1-C8 alkyl optionally substituted with one or more group(s) selected from halogen, cyano, C1-C3 alkyloxy, C1-C3 haloalkyloxy, 3- to 6-membered non-aromatic carbocyclyl optionally substituted with halogen, and 3- to 6-membered non-aromatic heterocyclyl optionally substituted with halogen;
or its pharmaceutically acceptable salt.
(3) The compound according to any one of the items (1), (2) and (1)′, wherein
A₁ is CR⁶ or N; A₂ is CR¹⁰ or N;
provided that when A₁ is N, then A₂ is CR¹⁰;
or its pharmaceutically acceptable salt.
(4) The compound according to any one of the items (1) to (3) and (1)′, wherein A₁ is CR⁶; A₂ is CR¹⁰; R⁶ is fluoro or chloro; and R¹⁰ is a hydrogen atom, or its pharmaceutically acceptable salt.
(4)′ The compound according to the item (1), wherein R⁶ is fluoro or chloro, or its pharmaceutically acceptable salt.
(5) The compound according to any one of the items (1) to (4), (1)′, and (4)′, wherein R⁵ is a hydrogen atom or fluoro, or its pharmaceutically acceptable salt.
(6) The compound according to any one of the items (1) to (5), (1)′, and (4)′, wherein R⁵ is a hydrogen atom, or its pharmaceutically acceptable salt.
(7) The compound according to any one of the items (1) to (6), (1)′, and (4)′, wherein

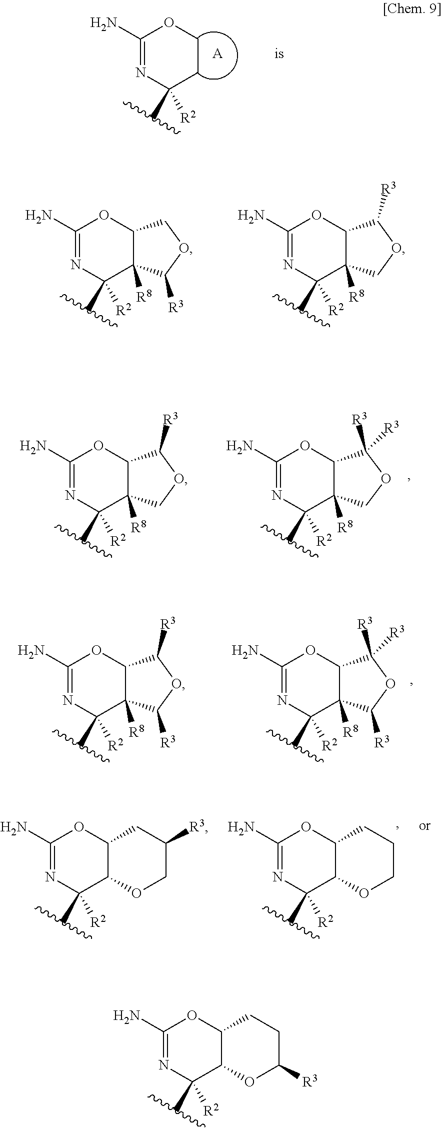

wherein each symbol is the same as defined in the item (1),
or its pharmaceutically acceptable salt.
(7′) The compound according to any one of the items (1) to (7), (1)′, and (4)′, wherein

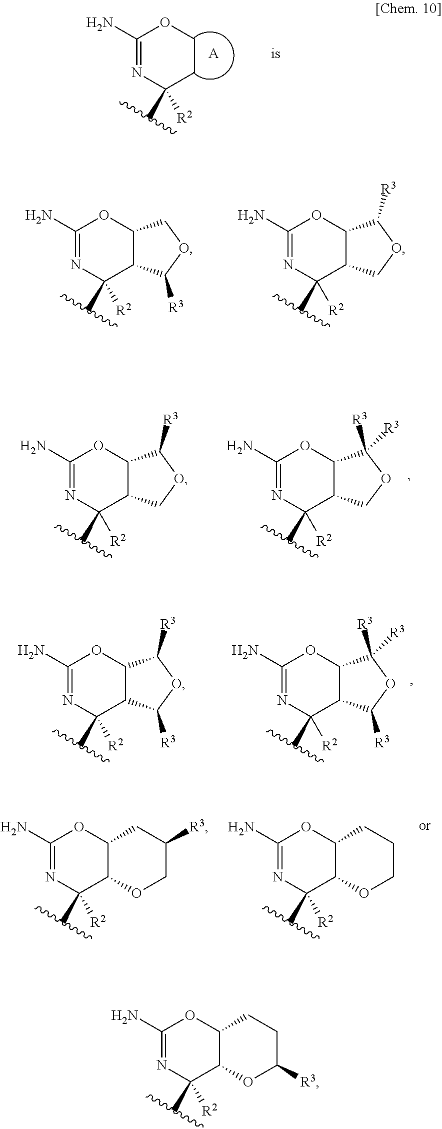

wherein each symbol is the same as defined in the item (1),
or its pharmaceutically acceptable salt.
(7-2) The compound according to any one of the items (1) to (7), (1)′, and (4)′, wherein

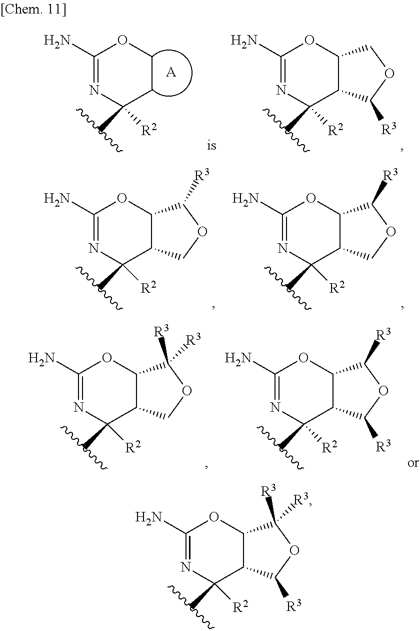

wherein each symbol is the same as defined in the item (1),
or its pharmaceutically acceptable salt.
(8) The compound according to any one of the items (1) to (7), (1)′, and (4)′, wherein

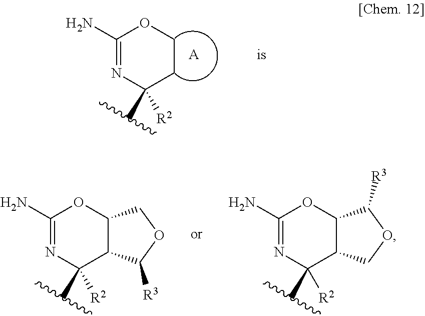

wherein each symbol is the same as defined in the item (1),
or its pharmaceutically acceptable salt thereof.
(9) The compound according to any one items (1) to (7), and (4)′, wherein

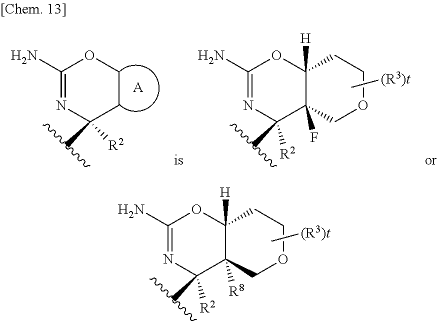

wherein each symbol is the same as defined in the item (1),
or its pharmaceutically acceptable salt.
(10) The compound according to any one of the items (1) to (9), (1)′, (4)′, (7)′, and (7-2), wherein R² is monofluoromethyl or difluoromethyl, or its pharmaceutically acceptable salt.
(11) The compound according to any one of the items (1) to (10), (1)′, (4)′, (7)′, and (7-2), wherein R³ is each independently C1-C6 alkyl optionally substituted with one or more substituents selected from the group consisting of halogen, cyano and alkyloxy, or a pharmaceutically acceptable salt thereof.
(12) The compound according to any one of the items (1) to (11), (1)′, (4)′, (7)′, and (7-2), wherein R³ is each independently methyl or C1-C6 alkyl substituted with fluoro, or a pharmaceutically acceptable salt thereof.
(13) The compound according to any one of the items (1) to (12), (1)′, (4)′, (7)′, and (7-2), wherein

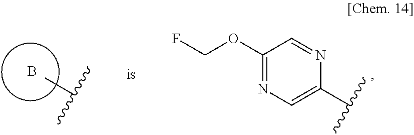

or its pharmaceutically acceptable salt.
(13-2) The compound according to any one of the item (1) to (12), (1)′, (4)′, (7)′, and (7-2), wherein

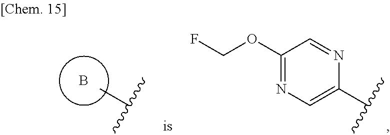

R² is monofluoromethyl,
R³ is methyl or C1-C6 alkyl substituted with fluoro,
R⁵ is a hydrogen atom, and
R⁶ is fluoro,
or its pharmaceutically acceptable salt.
(14) The compound according to the item (1), selected from the group consisting of Compound I-001, I-002, I-003, I-004, I-005, I-006, I-013, I-014, I-015, I-030, I-082, I-112, I-116 and I-117, or its pharmaceutically acceptable salt.
(14-2) The compound according to the item (1), selected from the group consisting of the compounds:

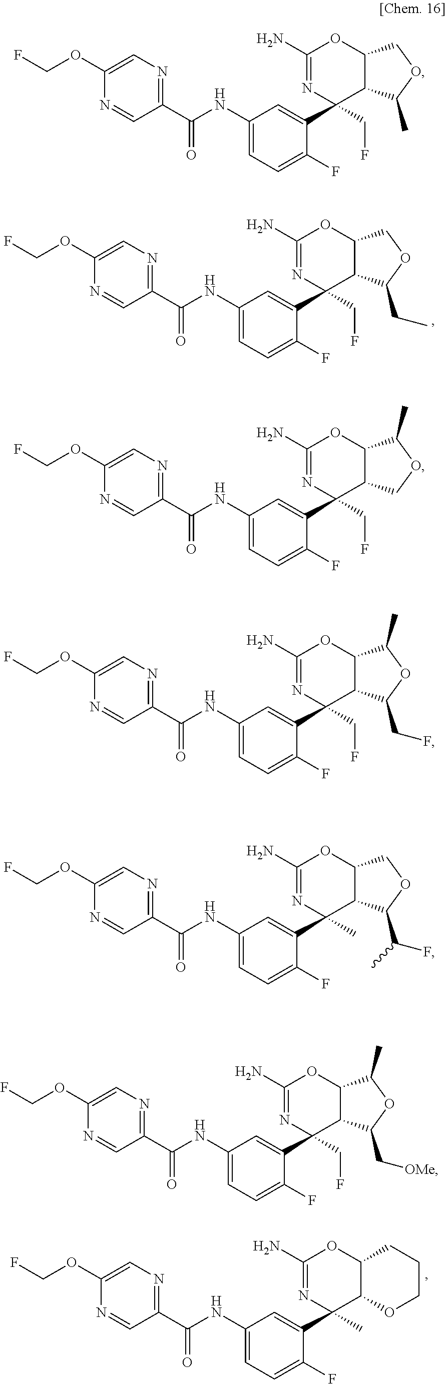

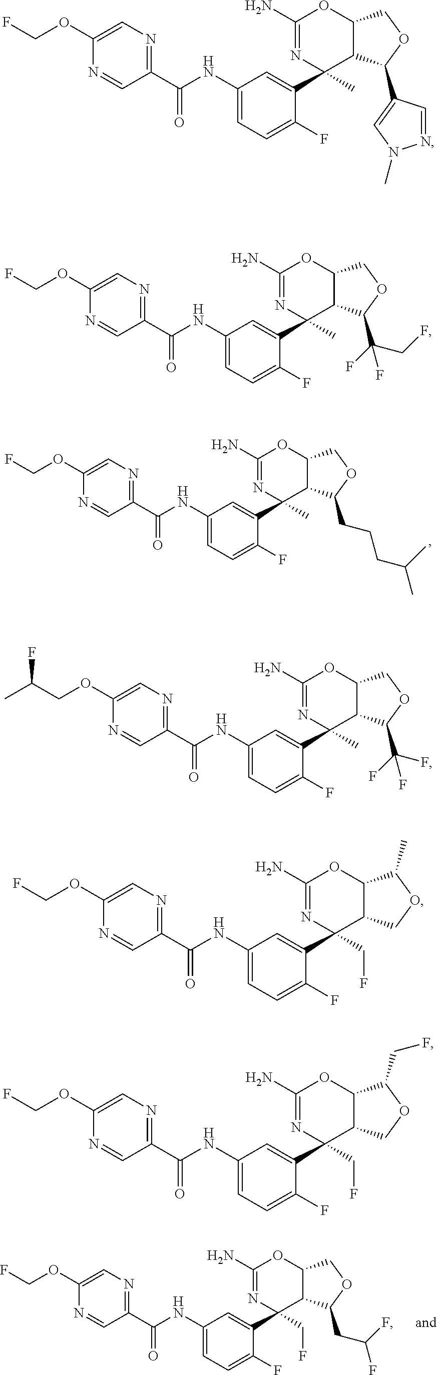

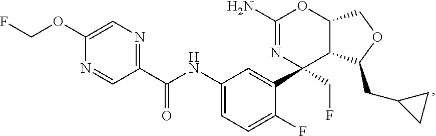 [SMILES: NC1=N[C@](CF)(c2cc(NC(=O)c3cnc(OCF)cn3)ccc2F)[C@H]2[C@H](CC3CC3)OC[C@H]2O1]

or its pharmaceutically acceptable salt.
(15) A pharmaceutical composition comprising the compound according to any one of the items (1) to (14), (1)′, (4)′, (7)′, (7-2), and (13-2), or its pharmaceutically acceptable salt.
(15-2) The pharmaceutical composition having BACE1 inhibitory activity comprising the compound according to the item (15), or a pharmaceutically acceptable salt thereof.
(16) The pharmaceutical composition according to the items (15) or (15-2), for treating or preventing Alzheimer dementia, mild cognitive impairment or prodromal Alzheimer's disease, for preventing the progression of Alzheimer dementia, mild cognitive impairment, or prodromal Alzheimer's disease, or for preventing the progression in a patient asymptomatic at risk for Alzheimer dementia.
(17) A compound according to any one of the items (1) to (14), (1)′, (4)′, (7)′, (7-2), (13-2), and (14-2), or its pharmaceutically acceptable salt for use in a method for inhibiting BACE1 activity.
(18) A compound according to any one of the items (1) to (14), (1)′, (4)′, (7)′, (7-2), (13-2), and (14-2), or its pharmaceutically acceptable salt for use in treating or preventing Alzheimer dementia, mild cognitive impairment or prodromal Alzheimer's disease, for use in preventing the progression of Alzheimer dementia, mild cognitive impairment or prodromal Alzheimer's disease, or for use in preventing the progression in a patient asymptomatic at risk for Alzheimer dementia.
(19) A method for inhibiting BACE1 activity comprising administering the compound according to any one of items (1) to (14), (1)′, (4)′, (7)′, (7-2), (13-2), and (14-2), or its pharmaceutically acceptable salt.
(20) A method for treating or preventing Alzheimer dementia, mild cognitive impairment or prodromal Alzheimer's disease, for preventing the progression of Alzheimer dementia, mild cognitive impairment, or prodromal Alzheimer's disease, or for preventing the progression in a patient asymptomatic at risk for Alzheimer dementia comprising administering the compound according to any one of items (1) to (14), (1)′, (4)′, (7)′, (7-2), (13-2), and (14-2), or its pharmaceutically acceptable salt.
(18) A BACE 1 inhibitor comprising the compound according to any one of items (1) to (14), (1)′, (4)′, (7)′, (7-2), (13-2), and (14-2), or its pharmaceutically acceptable salt.
(21) Use of the compound according to any one of items (1) to (14), (1)′, (4)′, (7)′, (7-2), (13-2), and (14-2), or its pharmaceutically acceptable salt for manufacturing a medicament for inhibiting BACE1 activity.
(22) The pharmaceutical composition according to the item (15) or (15-2) for treating or preventing a disease induced by production, secretion or deposition of amyloid β proteins.
(23) A method for treating or preventing diseases induced by production, secretion or deposition of amyloid β proteins comprising administering the compound according to any one of items (1) to (14), (1)′, (4)′, (7)′, (7-2), (13-2), and (14-2), or its pharmaceutically acceptable salt.
(24) A compound according to any one of items (1) to (14), (1)′, (4)′, (7)′, (7-2), (13-2), and (14-2), or its pharmaceutically acceptable salt for use in treating or preventing diseases induced by production, secretion or deposition of amyloid β proteins.
(25) Use of the compound according to any one of items (1) to (14), (1)′, (4)′, (7)′, (7-2), (13-2), and (14-2), or its pharmaceutically acceptable salt for manufacturing a medicament for treating or preventing diseases induced by production, secretion or deposition of amyloid β proteins.
26) The pharmaceutical composition according to the item (15) or (15-2), for treating or preventing Alzheimer dementia.
(27) A method for treating or preventing Alzheimer dementia comprising administering the compound according to any one of items (1) to (14), (1)′, (4)′, (7)′, (7-2), (13-2), and (14-2), or its pharmaceutically acceptable salt.
(28) A compound according to any one of items (1) to (14), (1)′, (4)′, (7)′, (7-2), (13-2), and (14-2), or its pharmaceutically acceptable salt for use in treating or preventing Alzheimer dementia.
(29) Use of the compound according to any one of items (1) to (14), (1)′, (4)′, (7)′, (7-2), (13-2), and (14-2), or its pharmaceutically acceptable salt for manufacturing a medicament for treating or preventing Alzheimer dementia.
(101) A pharmaceutical composition comprising the compound of any one of items (1) to (14), (1)′, (4)′, (7)′, (7-2), (13-2), and (14-2), or its pharmaceutically acceptable salt, for oral administration.
(102) The pharmaceutical composition of (101), which is a tablet, powder, granule, capsule, pill, film, suspension, emulsion, elixir, syrup, lemonade, spirit, aromatic water, extract, decoction or tincture.
(103) The pharmaceutical composition of (101) or (102), which is a sugar-coated tablet, film-coated tablet, enteric-coated tablet, sustained-release tablet, troche tablet, sublingual tablet, buccal tablet, chewable tablet, orally disintegrated tablet, dry syrup, soft capsule, micro capsule or sustained-release capsule.
(104) A pharmaceutical composition comprising the compound of any one of items (1) to (14), (1)′, (4)′, (7)′, (7-2), (13-2), and (14-2), or its pharmaceutically acceptable salt, for parenteral administration.
(105) The pharmaceutical composition of (104), for dermal, subcutaneous, intravenous, intraarterial, intramuscular, intraperitoneal, transmucosal, inhalation, transnasal, ophthalmic, inner ear or vaginal administration.
(106) The pharmaceutical composition of (104) or (105), which is injection, infusion, eye drop, nose drop, ear drop, aerosol, inhalation, lotion, impregnation, liniment, mouthwash, enema, ointment, plaster, jelly, cream, patch, cataplasm, external powder or suppository.
(107) A pharmaceutical composition comprising the compound of any one of items (1) to (14), (1)′, (4)′, (7)′, (7-2), (13-2), and (14-2), or its pharmaceutically acceptable salt, for a pediatric or geriatric patient.
(108) A pharmaceutical composition consisting of a combination of the compound of any one of items (1) to (14), (1)′, (4)′, (7)′, (7-2), (13-2), and (14-2), or its pharmaceutically acceptable salt and acetylcholinesterase inhibitor, NMDA antagonist, or other medicament for Alzheimer dementia.
(109) A pharmaceutical composition comprising the compound of any one of items (1) to (14), (1)′, (4)′, (7)′, (7-2), (13-2), and (14-2), or its pharmaceutically acceptable salt, for a combination therapy with acetylcholinesterase inhibitor, NMDA antagonist, or other medicament for Alzheimer dementia.

### DESCRIPTION OF EMBODIMENTS

Hereinafter, the present invention is described with reference to embodiments. It should be understood that, throughout the present specification, the expression of a singular form includes the concept of its plural form unless specified otherwise. Accordingly, it should be understood that an article in singular form (for example, in the English language, “a,” “an,” “the,” and the like) includes the concept of its plural form unless specified otherwise. Furthermore, it should be understood that the terms used herein are used in a meaning normally used in the art unless specified otherwise. Thus, unless defined otherwise, all technical and scientific terms used herein have the same meaning as those generally understood by those skilled in the art in the field to which the present invention pertains. If there is a contradiction, the present specification (including definitions) precedes.

Each meaning of terms used herein is described below. Both when used alone and in combination unless otherwise noted, each term is used in the same meaning.

In the specification, the term of “consisting of” means having only components.

In the specification, the term of “comprising” means not restricting with components and not excluding undescribed factors.

In the specification, the “halogen” includes fluorine, chlorine, bromine, and iodine. Fluorine and chlorine are preferable.

In the specification, the “alkyl” includes linear or branched alkyl of a carbon number of 1 to 15, for example, a carbon number of 1 to 10, for example, a carbon number of 1 to 6, and for example, a carbon number of 1 to 4. Examples include methyl, ethyl, n-propyl, isopropyl, n-butyl, isobutyl, sec-butyl, tert-butyl, n-pentyl, isopentyl, neopentyl, hexyl, isohexyl, n-heptyl, isoheptyl, n-octyl, isooctyl, n-nonyl and n-decyl. Examples are methyl, ethyl, n-propyl, isopropyl, n-butyl, isobutyl, sec-butyl, tert-butyl and n-pentyl.

In one embodiment, “alkyl” is methyl, ethyl, n-propyl, isopropyl or tert-butyl.

The term of “haloalkyl” includes a group wherein one or more hydrogen atoms attached to one or more carbon atoms of the above “alkyl” are replaced with one or more above “halogen”. Examples are monofluoromethyl, monofluoroethyl, monofluoropropyl, difluoromethyl, difluoroethyl, difluoropropyl, trifluoromethyl, trifluoroethyl, trifluoropropyl, pentafluoropropyl, monochloromethyl, monochloroethyl, monochloropropyl, dichloromethyl, dichloroethyl, dichloropropyl, trichloromethyl, trichloroethyl, trichloropropyl, pentachloropropyl, 1-fluoroethyl, 2-fluoroethyl, 1,1-difluoroethyl, 2,2-difluoroethyl, 2,2,2-trifluoroethyl, 1-chloroethyl, 2-chloroethyl, 1,1-dichloroethyl, 2,2-dichloroethyl, 2,2,2-trichloroethyl, 1,2-dibromoethyl, 1,1,1-trifluoropropan-2-yl and 2,2,3,3,3-pentafluoropropyl. Examples are monofluoromethyl, difluoromethyl, trifluoromethyl, 1-fluoroethyl, 1,1-difluoroethyl, and 2,2-difluoroethyl. Examples are monofluoromethyl, difluoromethyl, 1-fluoroethyl, 1,1-difluoroethyl and 2,2-difluoroethyl.

The term “alkenyl” includes linear or branched alkenyl of a carbon number or 2 to 15, for example, a carbon number of 2 to 10, for example, a carbon number of 2 to 6, and for example, a carbon number of 2 to 4, having one or more double bonds at any available positions. Examples include vinyl, allyl, propenyl, isopropenyl, butenyl, isobutenyl, prenyl, butadienyl, pentenyl, isopentenyl, pentadienyl, hexenyl, isohexenyl, hexadienyl, heptenyl, octenyl, nonenyl, decenyl, undecenyl, dodecenyl, tridecenyl, tetradecenyl and pentadecenyl. Examples are vinyl, allyl, propenyl, isopropenyl and butenyl.

The term “alkynyl” includes a linear or branched alkynyl of a carbon number of 2 to 15, for example, a carbon number of 2 to 10, for example, a carbon number of 2 to 8, for example, a carbon number of 2 to 6, and for example, a carbon number of 2 to 4 having one or more triple bonds at optionally positions. Specific examples are ethynyl, propynyl, butynyl, pentynyl, hexynyl, heptynyl, octynyl, nonynyl and decynyl. These may have further a double bond at any available position. Examples are ethynyl, propynyl, butynyl and pentynyl.

The term “alkylene” include a linear or branched divalent carbon chain of a carbon number of 1 to 15, for example, a carbon number of 1 to 10, for example, a carbon number of 1 to 6, and for example a carbon number of 1 to 3. Examples are methylene, dimethylene, and trimethylene.

One or more hydrogens of the alkylene in a compound of formula (I) can be replaced with an isotope of hydrogen ²H (deuterium).

The term of “alkyloxy” includes a group wherein an oxygen atom is substituted with the above “alkyl”. Examples are methoxy, ethoxy, n-propyloxy, isopropyloxy, n-butyloxy, tert-butyloxy, isobutyloxy, sec-butyloxy, pentyloxy, isopentyloxy and hexyloxy.

In one embodiment, “alkyloxy” is methoxy, ethoxy, n-propyloxy, isopropyloxy or tert-butyloxy.

The term of “alkenyloxy” includes a group wherein an oxygen atom is substituted with the above “alkenyl”. Examples are vinyloxy, allyloxy, 1-propenyloxy, 2-butenyloxy, 2-pentenyloxy, 2-hexenyloxy, 2-heptenyloxy and 2-octenyloxy.

The term of “alkynyloxy” includes a group wherein an oxygen atom is substituted with the above “alkynyl”. Examples are ethynyloxy, 1-propynyloxy, 2-propynyloxy, 2-butynyloxy, 2-pentynyloxy, 2-hexynyloxy, 2-heptynyloxy and 2-octynyloxy.

In one embodiment, “alkynyloxy” is ethynyloxy, 1-propynyloxy, and 2-propynyloxy.

The term of “carbocycle” includes non-aromatic carbocycle and aromatic carbocycle.

The term of “non-aromatic carbocycle” includes saturated carbocycle or unsaturated non-aromatic carbocycle which is monocyclic or which consists of two or more rings. A “non-aromatic carbocycle” of two or more rings includes a fused cyclic group wherein a non-aromatic monocyclic carbocycle or a non-aromatic carbocycle of two or more rings is fused with a ring of the above “aromatic carbocycle”.

In addition, the “non-aromatic carbocycle” also includes a cyclic group having a bridge or a cyclic group to form a spiro ring as follows:

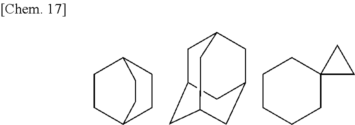

The term “non-aromatic monocyclic carbocycle” includes a group having 3 to 16 carbon atoms, for example, 3 to 12 carbon atoms, for example, 3 to 8 carbon atoms, and for example, 3 to 5 carbon atoms. Examples are cyclopropane, cyclobutane, cyclopentane, cyclohexane, cycloheptane, cyclooctane, cyclononane, cyclodecane, cyclopropenane, cyclobutenane, cyclopentenane, cyclohexenane, cycloheptenane and cyclohexadienane.

Examples of non-aromatic carbocycle consisting of two or more rings include a group having 6 to 14 carbon atoms, and examples are indane, indenane, acenaphthalene, tetrahydronaphthale and fluorenane.

The term of “aromatic carbocycle” includes an aromatic hydrocarbon ring which is monocyclic or which consists of two or more rings. Examples are an aromatic hydrocarbon group of a carbon number of 6 to 14, and specific examples are benzene, naphthalene, anthracene and phenanthrene.

In one embodiment, “aromatic carbocycle” is benzene.

In one embodiment, “carbocycle” is cyclopropane, cyclobutane and cyclopentane.

The term of “heterocycle” includes non-aromatic heterocycle and aromatic heterocycle.

The term of “non-aromatic heterocycle” includes a non-aromatic group which is monocyclic, or which consists of two or more rings, containing one or more of heteroatoms selected independently from oxygen, sulfur and nitrogen atoms.

A “non-aromatic heterocycle” of two or more rings includes a fused cyclic group wherein non-aromatic monocyclic heterocycle or non-aromatic heterocycle of two or more rings is fused with a ring of the above “aromatic carbocycle”, “non-aromatic carbocycle” and/or “aromatic heterocycle”.

In addition, the “non-aromatic heterocycle” also includes a cyclic ring having a bridge or a cyclic group to form a spiro ring as follows:

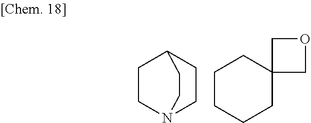

The term “non-aromatic monocyclic heterocycle” includes a 3- to 8-membered ring, and for example, 4-, 5- or 6-membered ring. Examples are dioxane, thiirane, oxirane, oxetane, oxathiolane, azetidine, thiane, thiazolidine, pyrrolidine, pyrroline, imidazolidine, imidazoline, pyrazolidine, pyrazoline, piperidine, piperazine, morpholinyl, morpholine, thiomorpholine, dihydropyridine, tetrahydropyridine, tetrahydrofurane, tetrahydropyrane, dihydrothiazoline, tetrahydrothiazoline, tetrahydroisothiazoline, dihydrooxazine, hexahydroazepine, tetrahydrodiazepine, tetrahydropyridazine, hexahydropyrimidine, dioxolane, dioxazine, aziridine, dioxoline, oxepane, thiolane, thiine and thiazine.

Examples of non-aromatic heterocycle of two or more rings includes a 9 to 14-membered group, and examples are indoline, isoindoline, chromane and isochromane.

The term of “aromatic heterocycle” includes an aromatic ring which is monocyclic, or which consists of two or more rings, containing one or more of heteroatoms selected independently from oxygen, sulfur and nitrogen atoms.

An “aromatic heterocycle” of two or more rings includes a fused cyclic group wherein aromatic monocyclic heterocyclyl or non-aromatic heterocycle consisting of two or more rings is fused with a ring of the above “aromatic carbocycle”.

The term “aromatic monocyclic heterocycle” includes a 5- to 8-membered group, and for example, 5- to 6-membered ring. Examples are pyrrole, imidazole, pyrazole, pyridine, pyridazine, pyrimidine, pyrazine, triazole, triazine, tetrazole, furane, thiophene, isoxazole, oxazole, oxadiazole, isothiazole, thiazole and thiadiazole.

Examples of aromatic bicyclic heterocycle includes a 9- to 10-membered ring, and examples are indoline, isoindoline, indazoline, indolizine, quinoline, isoquinoline, cinnoline, phthalazine, quinazoline, naphthyridine, quinoxaline, purine, pteridine, benzimidazole, benzisoxazole, benzoxazole, benzoxadiazole, benzisothiazole, benzothiazole, benzothiadiazole, benzofurane, isobenzofurane, benzothiophene, benzotriazole, imidazopyridine, triazolopyridine, imidazothiazole, pyrazinopyridazine, oxazolopyridine and thiazolopyridine.

Examples of aromatic heterocycle of three or more rings includes a 13 to 14-membered group, and examples are carbazole, acridine, xanthene, phenothiazine, phenoxathiine phenoxazine and dibenzofurane.

In one embodiment, “hetelocycle” is 1,4-oxathiane.

A “non-aromatic carbocyclyl” and “non-aromatic heterocyclyl” can be substituted with “oxo”. A group wherein two hydrogen atoms attached to the same carbon atom are replaced with oxo as follows is included:

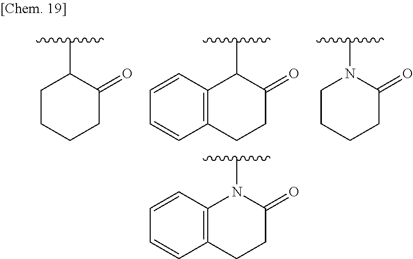

In the following formula, R⁸, R⁹ and “H” which are attached to bridgehead carbons are not substituted with R³.

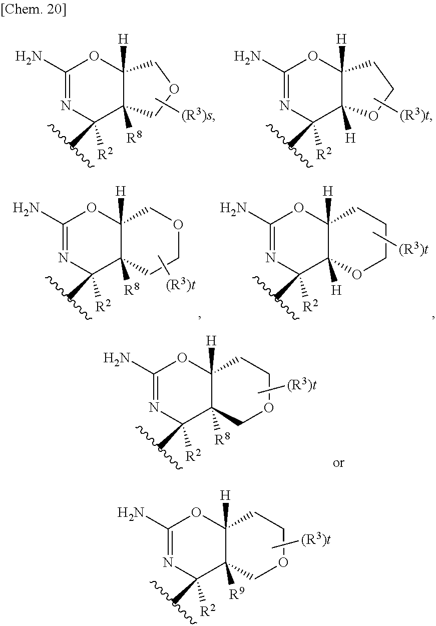

Specific embodiments of each symbol of the formula (I), and (I′) are illustrated bellow.

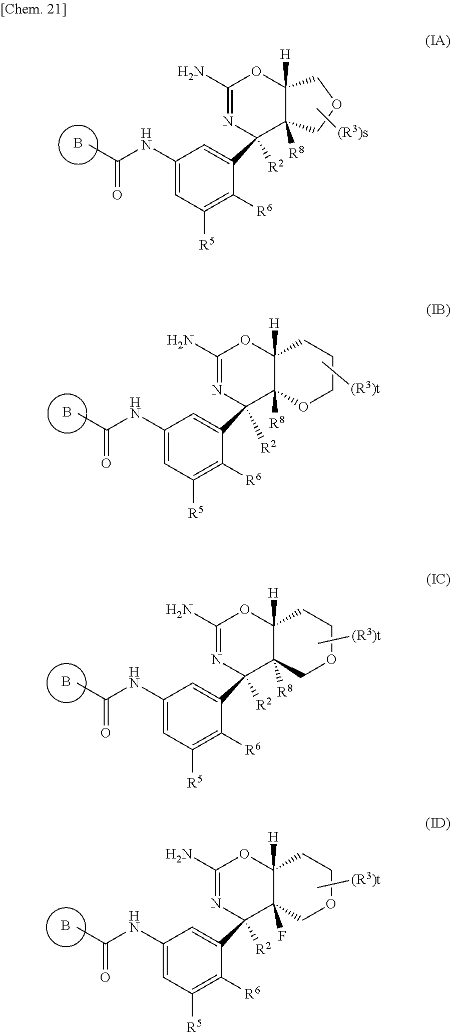

wherein each symbol is the same as defined above.

Specific embodiments of each symbol of the formula (I), (I′), (IA), (IB), (IC), and (ID) are illustrated below. All possible combinations of these embodiments are examples of the compounds of formulas (I), (I′), (IA), (IB), (IC) and (ID).

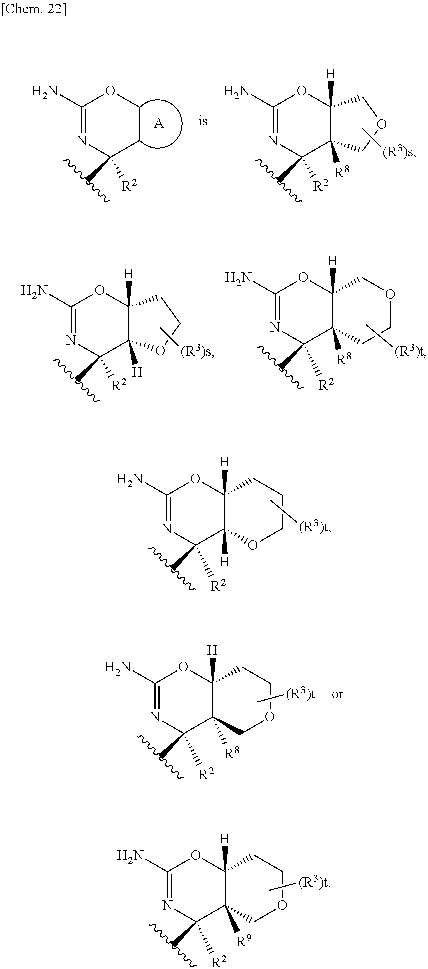

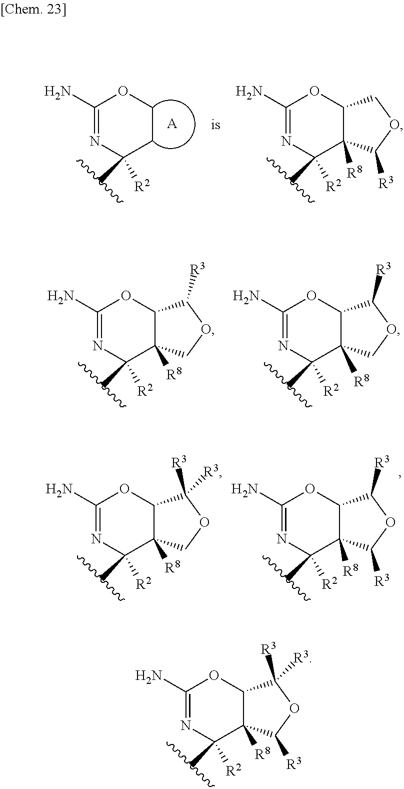

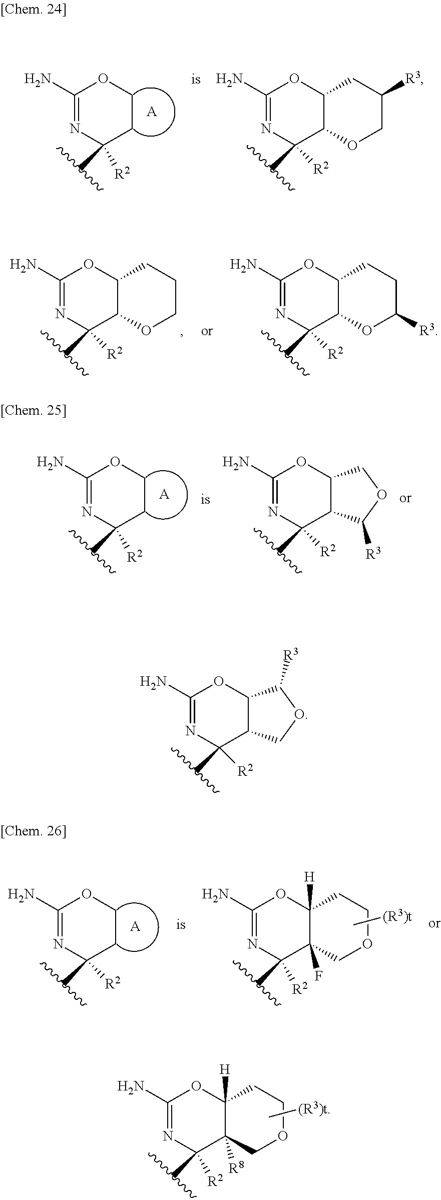

R² is C1-C3 alkyl optionally substituted with halogen.
R² is methyl optionally substituted with halogen.
R² is C1-C3 alkyl substituted with halogen.
R² is monofluoromethyl or difluoromethyl.
R² is monofluoromethyl.
R² is methyl.
R³ is each independently C1-C8 alkyl optionally substituted with one or more group(s) selected from halogen, cyano, C1-C3 alkyloxy, C1-C3 haloalkyloxy, 3- to 6-membered non-aromatic carbocyclyl optionally substituted with halogen, and 3- to 6-membered non-aromatic heterocyclyl optionally substituted with halogen;
aromatic heterocyclyl optionally substituted with one or more group(s) selected from C1-C3 alkyl and C1-C3 haloalkyl; or halogen.
R³ is each independently C1-C6 alkyl optionally substituted with one or more group(s) selected from halogen, cyano, C1-C3 alkyloxy, C1-C3 haloalkyloxy, 3- to 6-membered non-aromatic carbocyclyl optionally substituted with halogen, and 3- to 6-membered non-aromatic heterocyclyl optionally substituted with halogen.
R³ is each independently C1-C6 alkyl optionally substituted with one or more substituents selected from the group consisting of halogen, cyano and alkyloxy.
R³ is each independently C1-C3 alkyl or C1-C3 haloalkyl.
R³ is each independently C1-C3 alkyl.
R³ is each independently C1-C3 haloalkyl.
R³ is each independently methyl or ethyl.
R³ is each independently methyl.
s is an integer from 0 to 3.
s is an integer from 1 to 3.
s is 1 or 2.
s is 1.
t is an integer from 0 to 3.
t is an integer from 1 to 3.
t is 1 or 2.
t is 1.
t is 0 or 1.
R⁵ is a hydrogen atom or halogen.
R⁵ is a hydrogen atom.
R⁵ is fluoro.
A₁ is CR⁶ or N; and A₂ is CR¹⁰ or N.
A₁ is CR⁶ or N; and A₂ is CR¹⁰ or N, provided that when A₁ is N, then A₂ is CR¹⁰.
A₁ is CR⁶; and A₂ is CR¹⁰.
A₁ is CH; and A₂ is CF.
R⁶ is a hydrogen atom, halogen, or C1-C3 alkyl optionally substituted with halogen.
R⁶ is a hydrogen atom or halogen.
R⁶ is fluoro or chloro.
R⁶ is fluoro.
R¹⁰ is a hydrogen atom, halogen, or C1-C3 alkyl optionally substituted with halogen.
R¹⁰ is a hydrogen atom or halogen.
R¹⁰ is a hydrogen atom.

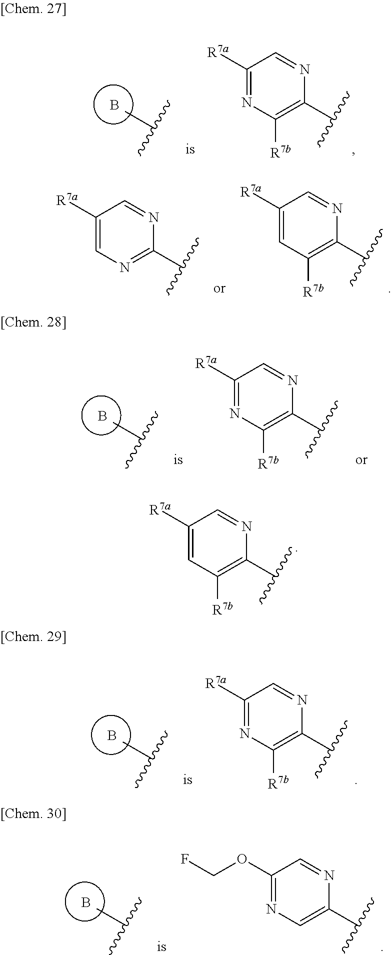

R^{7a} is halogen; cyano; C1-C6 alkyloxy optionally substituted with one or more group(s) selected from cyano, halogen, hydroxy, non-aromatic carbocyclyl and aromatic heterocyclyl; C1-C6 alkyl optionally substituted with one or more group(s) selected from cyano, halogen, and hydroxy; non-aromatic carbocyclyl optionally substituted with one or more group(s) selected from cyano and halogen; non-aromatic heterocyclyl optionally substituted with one or more group(s) selected from cyano and aromatic heterocyclyl; C1-C6 alkenyloxy optionally substituted with one or more group(s) selected from cyano, halogen, and hydroxy; C1-C6 alkynyloxy optionally substituted with one or more group(s) selected from cyano, halogen, and hydroxy; or aromatic heterocyclyl optionally substituted with one or more group(s) selected from C1-C6 alkyl.
R^{7a} is cyano; C1-C6 alkyloxy optionally substituted with cyano or halogen; or aromatic heterocyclyl.
R^{7a} is cyano; or C1-C3 alkyloxy optionally substituted with halogen.
R^{7a} is C1-C3 haloalkyloxy.
R^{7a} is cyano.

### R^{7a} is

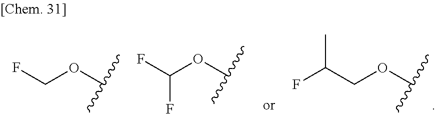

R^{7b} is a hydrogen atom, halogen, C1-C3 alky optionally substituted with halogen, or amino.
R^{7b} is a hydrogen atom or halogen.
R^{7b} is a hydrogen atom.
R⁸ is a hydrogen atom or halogen.
R⁸ is a hydrogen atom.
R⁸ is a fluoro.
R⁹ is halogen.
R⁹ is fluoro.

The compound of formula (I) is not limited to a specific isomer, and includes all possible isomers such as keto-enol isomers, imine-enamine isomers, diastereoisomers, optical isomers and rotation isomers, racemate and the mixture thereof. For example, the compound of formula (I) includes the following tautomers.

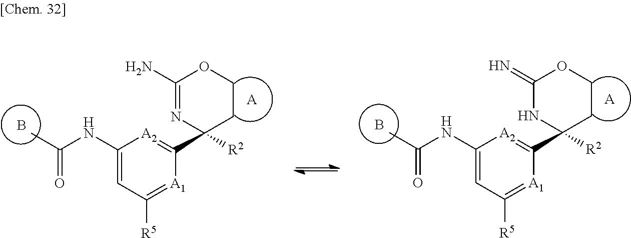

One or more hydrogen, carbon and/or other atoms of a compound of formula (I) can be replaced with an isotope of hydrogen, carbon and/or other atoms, respectively. Examples of isotopes include isotopes of hydrogen, carbon, nitrogen, oxygen, phosphorous, sulfur, fluorine, iodine and chlorine, such as ²H, ³H, ¹¹C, ¹³C, ¹⁴C, ¹⁵N, ¹⁸O, ¹⁷O, ³¹P, ³²P, ³⁵S, ¹⁸F, ¹²³I and ³⁶Cl respectively. The compound of formula (I) also includes the compound replaced with such isotopes. The compound replaced with such isotopes is useful also as a medicament, and includes all the radiolabeled compounds of the compound of formula (I). The invention includes “radiolabelling method” for manufacturing the “radiolabeled compound” and the method is useful as a tool of metabolic pharmacokinetic research, the research in binding assay and/or diagnosis. A radiolabeled compound of the compound of formula (I) can be prepared by methods known in the art. For example, tritiated compounds of formula (I) can be prepared by introducing tritium into the particular compound of formula (I) such as by catalytic dehalogenation with tritium. This method may include reacting a suitably halogenated precursor of a compound of formula (I) with tritium gas in the presence of a suitable catalyst such as Pd/C, in the presence or absence of a base. Other suitable methods for preparing tritiated compounds can be found in Isotopes in the Physical and Biomedical Sciences, Vol. 1, Labeled Compounds (Part A), Chapter 6 (1987). A ¹⁴C-labeled compound can be prepared by employing starting materials having ¹⁴C carbon.

As pharmaceutically acceptable salt of the compound of formula (I), examples include salts with alkaline metals (e.g. lithium, sodium and potassium), alkaline earth metals (e.g. calcium and barium), magnesium, transition metal (e.g. zinc and iron), ammonia, organic bases (e.g. trimethylamine, triethylamine, dicyclohexylamine, ethanolamine, diethanolamine, triethanolamine, meglumine, diethanolamine, ethylenediamine, pyridine, picoline, quinoline), and amino acids, and salts with inorganic acids (e.g. hydrochloric acid, sulfuric acid, nitric acid, carbonic acid, hydrobromic acid, phosphoric acid and hydroiodic acid) and organic acids (e.g. formic acid, acetic acid, propionic acid, trifluoroacetic acid, citric acid, lactic acid, tartaric acid, oxalic acid, maleic acid, fumaric acid, succinic acid, mandelic acid, glutaric acid, malic acid, benzoic acid, phthalic acid, ascorbic acid, benzenesulfonic acid, p-toluenesulfonic acid, methanesulfonic acid and ethanesulfonic acid). Specific Examples are salts with hydrochloric acid, sulfuric acid, phosphoric acid, tartaric acid, or methanesulfonic acid. These salts can be formed by the usual method.

The compounds of the present invention represented by formula (I) or pharmaceutically acceptable salts thereof may form solvates (e.g., hydrates etc.) and/or crystal polymorphs. The present invention encompasses those various solvates and crystal polymorphs. “Solvates” may be those wherein any number of solvent molecules (e.g., water molecules etc.) are coordinated with the compounds represented by formula (I). When the compounds represented by formula (I) or pharmaceutically acceptable salts thereof are allowed to stand in the atmosphere, the compounds may absorb water, resulting in attachment of adsorbed water or formation of hydrates. Recrystallization of the compounds represented by formula (I) or pharmaceutically acceptable salts thereof may produce crystal polymorphs.

The compounds of the present invention represented by formula (I) or pharmaceutically acceptable salts thereof may form prodrugs. The present invention also encompasses such various prodrugs. Prodrugs are derivatives of the compounds of the present invention that have chemically or metabolically degradable groups and are compounds that are converted to the pharmaceutically active compounds of the present invention through solvolysis or under physiological conditions in vivo. Prodrugs include compounds that are converted to the compounds represented by formula (I) through enzymatic oxidation, reduction, hydrolysis and the like under physiological conditions in vivo and compounds that are converted to the compounds represented by formula (I) through hydrolysis by gastric acid and the like. Methods for selecting and preparing suitable prodrug derivatives are described, for example, in the Design of Prodrugs, Elsevier, Amsterdam 1985. Prodrugs themselves may be active compounds.

When the compounds of formula (I) or pharmaceutically acceptable salts thereof have a hydroxy group, prodrugs include acyloxy derivatives and sulfonyloxy derivatives which can be prepared by reacting a compound having a hydroxy group with a suitable acid halide, suitable acid anhydride, suitable sulfonyl chloride, suitable sulfonylanhydride and mixed anhydride or with a condensing agent. Examples are CH₃COO—, C₂H₅COO—, t-BuCOO—, C₁₅H₃₁COO—, PhCOO—, (m-NaOOCPh)COO—, NaOOCCH₂CH₂COO—, CH₃CH(NH₂)COO—, CH₂N(CH₃)₂COO—, CH₃SO₃—, CH₃CH₂SO₃—, CF₃SO₃—, CH₂FSO₃—, CF₃CH₂SO₃—, p-CH₃—O-PhSO₃—, PhSO₃— and p-CH₃PhSO₃—.

The compounds of formula (I) may be prepared by the methods described below, together with synthetic methods known to a person skilled in the art.

The starting materials are commercially available or may be prepared in accordance with known methods.

During any of the following synthesis, it may be necessary or preferable to protect sensitive or reactive groups on any of molecules. In such case, these protections can be achieved by means of conventional protective groups such as those described in Greene's Protective Group in Organic Synthesis, John Wily & Sons, 2007.

It will be understood by a person skilled in the art that the compounds described below will be generated as a mixture of diastereomers and/or enantiomers, which may be separated at relevant stages of the following procedures using conventional techniques such as crystallization, silica gel chromatography, chiral or achiral high performance liquid chromatography (HPLC), and chiral supercritical fluid (SFC) chromatography to provide the single enantiomers of the invention.

During all the following steps, the order of the steps to be performed may be appropriately changed. In each step, an intermediate may be isolated and then used in the next step. All of reaction time, reaction temperature, solvents, reagents, and protecting groups, etc. are mere exemplification and not limited as long as they do not cause an adverse effect on a reaction.

### General Procedure A

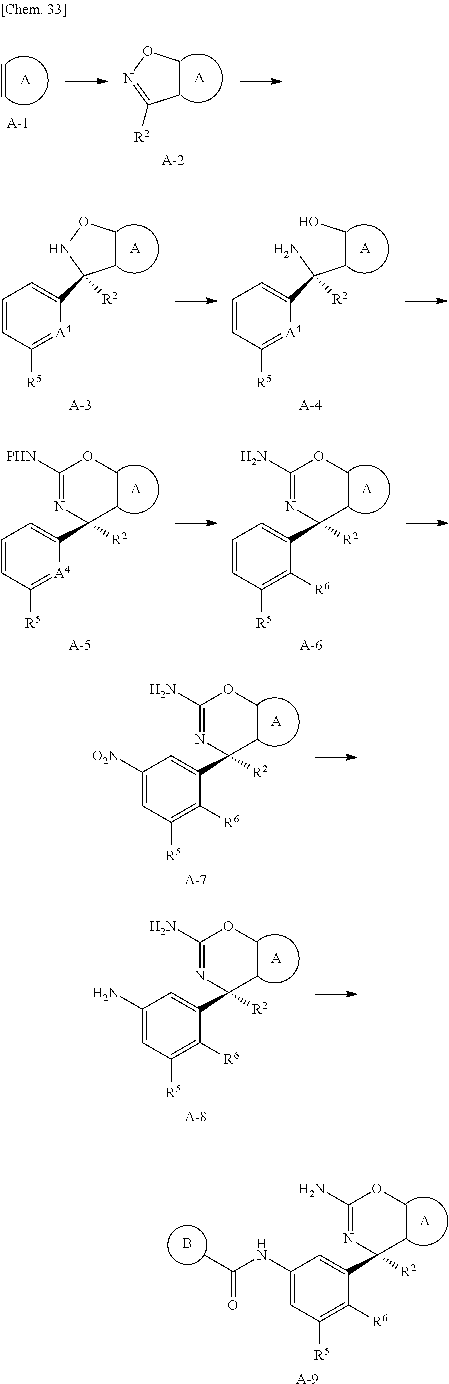

Wherein P is a protective group such as benzoyl or benzyl and the other symbols are the same as defined above (1).

General Procedure A is a method for preparing compounds of Compound A-9 from Compounds A-1 through multiple steps of Step 1 to Step 8. Those skilled in the art will be appreciate that protective groups P can be chosen depending on the reaction conditions used in later steps.

### Step 1

Compound A-2 can be prepared by means of 1,3-dipolar cycloaddition. This type of reactions can be conducted using similar conditions described in J. Am. Chem. Soc., 1960, 82, 5339-5342 or J. Org. Chem. 1998, 63, 5272-5274. This 1,3-dipolar cycloaddition can be conducted with cyclic Compound A-1 and the corresponding nitrile oxides generated in situ from the corresponding nitroalkanes using an appropriate dehydrating agents such as, for example, phenyl isocyanate, phenyl diisocyanate or (Boc)₂O, and an appropriate base such as, for example, triethylamine, dipropylethylamine or N-methylmorpholine. Alternatively, the nitrile oxides can be generated in situ from the corresponding hydroximoly chlorides with an appropriate base such as, for example, triethylamine, dipropylethylamine or N-methylmorpholine. The solvent used in this step is not particularly limited in so far as it does not interfere with the reaction. Examples of the solvent include tetrahydrofuran, 1,4-dioxane, 1,2-dimethoxyethane, diethyl ether, toluene, benzene, and mixed solvents thereof. The reaction temperature is preferably room temperature to 120° C. The reaction time is not particularly limited and is usually 5 minutes to 24 hours, preferably 30 minutes to 24 hours.

### Step 2

Compound A-3 can be prepared by means of the nucleophilic addition of an appropriate aryllithium reagents or Grignard reagents to Compound A-2. This type of reactions can be conducted using similar conditions described in J. Am. Chem. Soc., 2005, 127, 5376-5384. Preferably, the aryllithium reagents or Grignard reagents can be prepared from the corresponding aromatic halides using an appropriate base, such as, for example, n-, sec- or tert-butyl lithium, isopropylmagnesium bromide or metallic magnesium, which can be then reacted to Compound A-2 with Lewis acid such as, for example, BF₃-OEt₂ to give Compound A-3. The solvent used in this step is not particularly limited in so far as it does not interfere with the reaction. Examples of the solvent include tetrahydrofuran, 1,4-dioxane, 1,2-dimethoxyethane, diethyl ether, toluene, benzene, and mixed solvents thereof. The reaction temperature is preferably −78° C. to room temperature. The reaction time is not particularly limited and is usually 5 minutes to 24 hours, preferably 30 minutes to 24 hours.

### Step 3

Compound A-4 can be prepared by reductive cleavage reaction of the N—O bond of compound A-3. This reductive cleavage can be conducted using zinc with an appropriate acid such as acetic acid, formic acid or hydrochloric acid. The solvent used in this step is not particularly limited in so far as it does not interfere with the reaction. Examples of the solvent include methanol, ethanol, tetrahydrofuran, water and mixed solvents thereof. The reaction temperature is preferably −20° C. to solvent reflux temperature. The reaction time is not particularly limited and is usually 5 minutes to 24 hours, preferably 30 minutes to 24 hours.

Alternatively, this reaction can be performed using a metal catalyst such as platinum oxide under hydrogene. The solvent used in this step is not particularly limited in so far as it does not interfere with the reaction. Examples of the solvent include methanol, ethanol, water and mixed solvents thereof. The reaction temperature is preferably room temperature to 50° C. The reaction time is not particularly limited and is usually 5 minutes to 24 hours, preferably 30 minutes to 24 hours.

Furthermore, this type of reaction can also be conducted using lithium aluminum hydride. The solvent used in this step is not particularly limited in so far as it does not interfere with the reaction. Examples of the solvent include tetrahydrofuran, 1,4-dioxane, 1,2-dimethoxyethane, diethyl ether and mixed solvents thereof. The reaction temperature is preferably −20° C. to room temperature. The reaction time is not particularly limited and is usually 5 minutes to 24 hours, preferably 30 minutes to 24 hours.

### Step 4

Compound A-5 can be prepared by formation of the corresponding thioureas from Compound A-4 in situ, followed by cyclization reaction. This type of reactions is known to a person skilled in the art and can be performed under the conditions described in WO2014/065434. The thiourea can be obtained in situ from Compound A-4 using an appropriate isothiocyanates such as, for example benzoyl isothiocyanate or benzyl isothiocyanate, then cyclization can be performed by adding reagents such as, for example m-CPBA, hydrogene peroxide, or carbodiimide reagents (e. g. DCC, DIC or EDC). Alternatively, this cyclization can be performed using alkylating reagents such as methyl iodide, and an appropriate base such as sodium hydride, sodium bicarbonate and potassium carbonate. The solvent used in this step is not particularly limited in so far as it does not interfere with the reaction. Examples of the solvent include chloroform, dichloromethane, dichloroethane, tetrahydrofuran, and mixed solvents thereof. The reaction temperature is usually 0° C. to 60° C. The reaction time is not particularly limited and is usually 5 minutes to 24 hours, preferably 30 minutes to 24 hours.

### Step 5

Compound A-6 can be prepared by deprotection of Compound A-5. This deprotection reaction is known to a person skilled in the art and can be performed under the conditions described in Green's Protective Groups in Organic Synthesis, 4^{th} ed. When the protecting group is benoyl, the deprotecting reaction can be conducted under acidic conditions such as sulfuric acid or hydrochloric acid, or under basic condition such as hydrazine, DBU, or sodium hydroxide. The solvent used in this step is not particularly limited in so far as it does not interfere with the reaction. Examples of the solvent include dichloromethane, tetrahydrofuran, 1,4-dioxane, methanol, toluene, benzene and mixed solvents thereof. The reaction temperature is preferably room temperature to 100° C. The reaction time is not particularly limited and is usually 5 minutes to 24 hours, preferably 30 minutes to 24 hours.

### Step 6

Compound A-7 can be prepared by nitration of Compound A-6. A typical procedure involves the treatment of Compound A₆ dissolved in sulfuric acid and trifluoroacetic acid, with a source of nitronium ion, such as, for example, potassium nitrate or nitric acid. The reaction temperature is preferably −20° C. to 0° C. The reaction time is not particularly limited and is usually 5 minutes to 5 hours, preferably 30 minutes to 2 hours.

### Step 7

Compound A-8 can be prepared by reduction of Compound A-7. The reduction can be conducted by a suitable catalyst, such as, for example, palladium on carbon under hydrogen atmosphere, or the use of a reducing agent such as, for example, iron, zinc or tin(II) chloride. The solvent used in this step is not particularly limited in so far as it does not interfere with the reaction. Examples of the solvent include tetrahydrofuran, methanol, ethanol, water, and mixed solvents thereof. The reaction temperature is usually room temperature to 80° C. and is preferably room temperature to 60° C. The reaction time is not particularly limited and is usually 5 minutes to 24 hours, preferably 30 minutes to 24 hours.

### Step 8

Compound A-9 can be prepared by amide coupling reaction of Compound A-8 with the corresponding carboxylic acids. This reaction can be conducted by a method known to a person skilled in the art, and suitable coupling conditions can be found in Chem. Rev. 2011, 111, 6557-6602, which includes: a) reactions using condensation reagents; b) reactions using acid chlorides or fluorides.

Reaction a) can be conducted by use of condensation reagents such as dicyclohexylcarbodiimide (DCC), diisopropylcarbodiimide (DIC), 1-ethyl-3-(3-dimethylaminopropyl) carbodiimide hydrochloride (EDC hydrochloride), O-(7-aza-1H-benzotriazol-1-yl)-N,N,N′,N′-tetramethyluronium hexafluorophosphate (HATU), and 1H-Benzotriazol-1-yloxy-tri(pyrrolidino) phosphonium hexafluorophosphate (PyBOP). When using uronium or phosphonium salts such as HATU and PyBOP, the reaction can be performed in the presence of bases such as triethylamine and diisopropylethylamine. The reaction may be accelerated by use of catalysts such as 1-hydroxy-benzotriazole (HOBt) and 1-hydroxy-7-aza-benzotriazole (HOAt). The solvent used in the reaction is not particularly limited in so far as it does not interfere with the reaction. Examples of the solvent include dichloromethane, N,N-dimethylformamide (DMF), N-methylpyrrolidone (NMP), and tetrahydrofuran. The reaction temperature is usually 0° C. to 50° C. and is preferably room temperature.

Reaction b) can be performed by use of commercially available acid chlorides or those synthesized by known methods to a person skilled in the art in solvents such as dichloromethane, tetrahydrofuran, and ethyl acetate in the presence of bases such as triethylamine, diisopropylethylamine, pyridine, and N,N-dimethyl-4-aminopyridine. The reaction temperature is usually 0° C. to 60° C. and is preferably 0° C. to room temperature. The reaction time is not particularly limited and is usually 5 minutes to 24 hours, preferably 20 minutes to 6 hours.

### General Procedure B

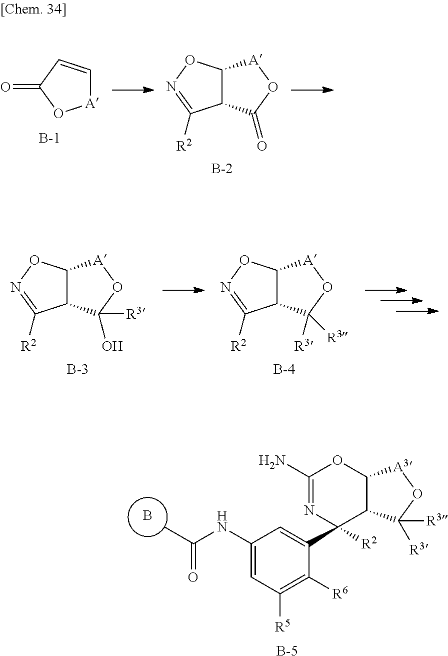

Wherein A′ is substituted or unsubstituted C1-2 alkylene, R³′ and R³″ are each independently selected from the group consisting of alkyl optionally substituted with halogen, cyano, alkyloxy, haloalkyloxy or non-aromatic calbocyclyl, and heteroaryl optionally substituted with alkyl, and other symbols are the same as defined above.

General Procedure B is a method for preparing Compound B-5 from Compound B-1 through multiple steps. Using Compound B-4 and Compound B-5 can be prepared according to the methods described in General procedure A.

### Step 1

Compound B-2 can be prepared by means of 1,3-dipolar cycloaddition. This type of reactions can be conducted using similar conditions described in J. Am. Chem. Soc. 1960, 82, 5339-5342 or J. Org. Chem. 1998, 63, 5272-5274. This 1,3-dipolar cycloaddition can be conducted with cyclic Compound B-1 and the corresponding nitrile oxides generated in situ from the corresponding nitroalkanes using an appropriate dehydrating agents such as, for example, phenyl isocyanate, phenyl diisocyanate or (Boc)₂O, and an appropriate base such as, for example, triethylamine, diisopropylethylamine or N-methylmorpholine. Alternatively, the nitrile oxides can be generated in situ from the corresponding hydroximoly chlorides with an appropriate base such as, for example, triethylamine, diisopropylethylamine or N-methylmorpholine. The solvent used in this step is not particularly limited in so far as it does not interfere with the reaction. Examples of the solvent include tetrahydrofuran, 1,4-dioxane, 1,2-dimethoxyethane, diethyl ether, toluene, benzene, and mixed solvents thereof. The reaction temperature is preferably room temperature to 120° C. The reaction time is not particularly limited and is usually 5 minutes to 24 hours, preferably 30 minutes to 24 hours.

### Step 2

When R³′ is a hydrogen atom, Compound B-3 can be prepared by carbonyl reduction of Compound B-2. This type of reactions can be conducted using an appropriate metal hydrides such as, for example, DIBAL-H, lithium tri-tert-butoxyaluminum hydride or sodium bis(2-methoxyethoxy)aluminum, by means of the nucleophilic addition to Compound B-2. The solvent used in this step is not particularly limited in so far as it does not interfere with the reaction. Examples of the solvent include dichloromethane, tetrahydrofuran, 1,4-dioxane, 1,2-dimethoxyethane, diethyl ether, toluene, benzene, and mixed solvents thereof. The reaction temperature is preferably −78° C. to room temperature. The reaction time is not particularly limited and is usually 5 minutes to 24 hours, preferably 30 minutes to 24 hours.

When R³′ is other than a hydrogen atom, Compound B-3 can be prepared by means of the nucleophilic addition to Compound B-2. This type of reactions can be conducted using an appropriate nucleophiles such as, for example organic llithium, magnesium, zinc or silyl reagents, with or without Leiws acid such as, for example BF₃-OEt₂, AlCl₃ or TiCl₄. The solvent used in this step is not particularly limited in so far as it does not interfere with the reaction. Examples of the solvent include dichloromethane, tetrahydrofuran, 1,4-dioxane, 1,2-dimethoxyethane, diethyl ether, toluene, benzene, and mixed solvents thereof. The reaction temperature is preferably −78° C. to room temperature. The reaction time is not particularly limited and is usually 5 minutes to 24 hours, preferably 30 minutes to 24 hours.

### Step 3

When R³″ is a hydrogen atom, Compound B-4 can be prepared by reduction of Compound B-3. This type of reactions can be conducted using an appropriate reducing agents such as triethylsilane, sodium borohydride with or without Leiws acid such as BF₃-OEt₂. The solvent used in this step is not particularly limited in so far as it does not interfere with the reaction. Examples of the solvent include dichloromethane, acetonitrile, tetrahydrofuran, 1,4-dioxane, 1,2-dimethoxyethane, diethyl ether, toluene, benzene, and mixed solvents thereof. The reaction temperature is preferably −20° C. to room temperature. The reaction time is not particularly limited and is usually 5 minutes to 24 hours, preferably 30 minutes to 24 hours.

When R³″ is other than a hydrogen atom, Compound B-4 can be prepared by means of the nucleophilic addition to Compound B-2. This type of reactions can be conducted using an appropriate nucleophiles such as, for example organic llithium, magnesium, zinc or silyl reagents, with or without Leiws acid such as, for example BF₃-OEt₂, AlCl₃ or TiCl₄. The solvent used in this step is not particularly limited in so far as it does not interfere with the reaction. Examples of the solvent include dichloromethane, tetrahydrofuran, 1,4-dioxane, 1,2-dimethoxyethane, diethyl ether, toluene, benzene, and mixed solvents thereof. The reaction temperature is preferably −78° C. to room temperature. The reaction time is not particularly limited and is usually 5 minutes to 24 hours, preferably 30 minutes to 24 hours.

### General Procedure C

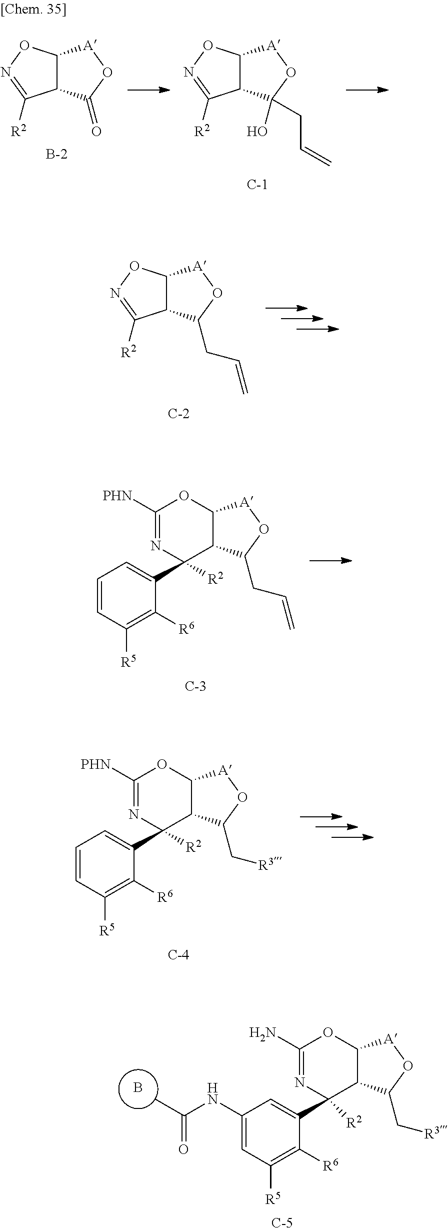

Wherein P is a protective group such as benzoyl or benzyl, R³′″ is ethyl or cyclopropyl, and the other symbols are the same as defined above (1).

General Procedure C is a method for preparing Compound C-5 from Compound B-1 through multiple steps. Compound C-3 and Compound C-5 can be prepared from Compound C-2 and C-5 according to the methods described in General procedure A.

### Step 1

Compound C-1 can be prepared by means of the nucleophilic addition of allyl moiety to carbonyl group of Compound B-2. This type of reactions can be conducted using an appropriate commercially available or in situ generated allyl reagents such as, for example allyl silane, llithium, magnesium, zinc reagents, with or without Leiws acid such as, for example BF₃-OEt₂, AlCl₃ or TiCl₄. The solvent used in this step is not particularly limited in so far as it does not interfere with the reaction. Examples of the solvent include dichloromethane, tetrahydrofuran, 1,4-dioxane, 1,2-dimethoxyethane, diethyl ether, toluene, benzene, and mixed solvents thereof. The reaction temperature is preferably −78° C. to room temperature. The reaction time is not particularly limited and is usually 5 minutes to 24 hours, preferably 30 minutes to 24 hours.

### Step 2

Compound C-2 can be prepared by reduction of Compound C-1. This type of reactions can be conducted using appropriate reducing agents such as triethylsilane or sodium borohydride, with or without Leiws acid such as BF₃-OEt₂. The solvent used in this step is not particularly limited in so far as it does not interfere with the reaction. Examples of the solvent include dichloromethane, acetonitrile, tetrahydrofuran, 1,4-dioxane, 1,2-dimethoxyethane, diethyl ether, toluene, benzene, and mixed solvents thereof. The reaction temperature is preferably −20° C. to room temperature. The reaction time is not particularly limited and is usually 5 minutes to 24 hours, preferably 30 minutes to 24 hours.

### Step 3

When R³′″ is ethyl, Compound C-5 can be obtained by hydrogenation of Compound C-4. The hydrogenation can be performed using suitable catalyst such as, for example palladium on carbon under hydrogene atmosphere. The solvent used in this step is not particularly limited in so far as it does not interfere with the reaction. Examples of the solvent include tetrahydrofuran, methanol, ethanol, water, and mixed solvents thereof. The reaction temperature is usually room temperature to 80° C. and is preferably room temperature to 60° C. The reaction time is not particularly limited and is usually 5 minutes to 24 hours, preferably 30 minutes to 24 hours.

When R³′″ is cyclopropyl, Compound C-5 can be obtained by means of cyclopropanation of Compound C-4. This type of reaction can be performed using an appropriate reagent such as diazomethane with or without a suitable catalyst, or Simmons-Smith reaction condition such as, for example diiodomethane with diethylzinc. The solvent used in this step is not particularly limited in so far as it does not interfere with the reaction. Examples of the solvent include dichloromethane, diethylether, toluene, benzene, or mixed solvents thereof. The reaction temperature is usually −30° C. to room temperature. The reaction time is not particularly limited and is usually 5 minutes to 24 hours, preferably 30 minutes to 24 hours.

### General Procedure D

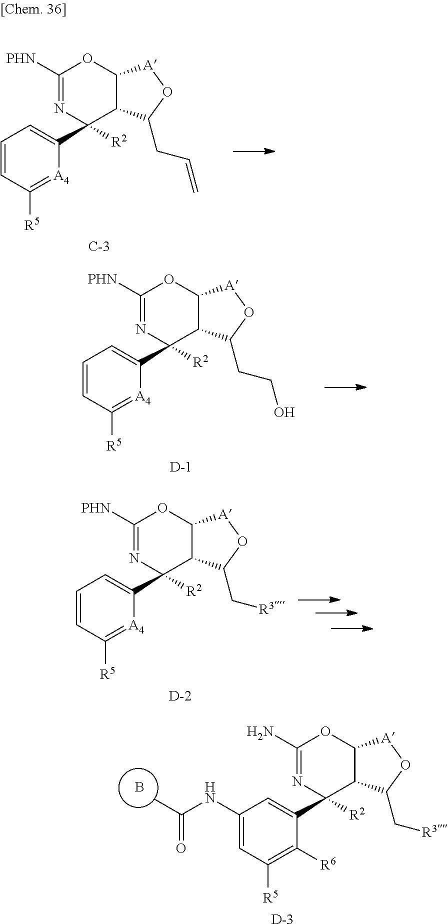

Wherein P is a protective group such as benzoyl or benzyl, R³″″ is alkyl substituted with fluorine or alkyloxy, and the other symbols are the same as defined above (1).

General Procedure D is a method for preparing compounds of Compound D-3 from Compound C-3 through multiple steps. Compound D-3 can be prepared from Compound D-2 according to the methods described in General procedure A.

### Step 1

Compound D-1 can be prepared by ozonolysis of Compound C-3, followed by reduction of the resulting aldehyde. This reaction can be performed by a method known to a person skilled in the art. The ozonolysis can be performed under ozone atmosphere in suitable solvent such as dichloromethane, methanol, and mixed thereof, with an appropriate regents such as triphenylphosphine, pyridine, dimethylsulfide and trimethylamine under nitrogene atmosphere for reductive workup. The temperature for generation of ozonide is preferably −78° C., then the temperature can be allowed to warm to room temperature for reductive workup. The reaction time is not particularly limited and is usually 30 minutes to 5 hours, preferably 30 minutes to 2 hours. The reduction of the resulting aldehyde can be performed in one pot using an appropriate reducing agent such as sodium borohydride or lithium aluminum hydride. The reaction temperature is preferably 0° C. to room temperature. The reaction time is not particularly limited and is usually 30 minutes to 5 hours, preferably 30 minutes to 2 hours.

### Step 2

When R³″″ is CF₃, CHF₂ or CH₂F, Compound D-2 can be obtained by two-step sequence; oxidation of Compound D-1 to the aldehyde or carboxylic acid followed by fluorination, or direct fluorination of Compound D-1. This reaction can be performed by a method known to a person skilled in the art. For example, Compound D-1 can be oxidized to the corresponding aldehyde under an appropriate oxidation condition such as, for example TEMPO, Dess-Martin or Swern oxidation. The corresponding carboxylic acid can be obtained by oxidation of the resulting aldehyde, or oxidizing Compound D-1 directly using an appropriate condition such as for example, Pinnick, TEMPO or Jonse oxadation. The solvent used in this step is not particularly limited in so far as it does not interfere with the reaction. The reaction temperature is usually −78° C. to room temperature. The reaction time is not particularly limited and is usually 5 minutes to 24 hours, preferably 30 minutes to 24 hours. The flurorination reaction can be performed using an appropriate reagent such as, for example DAST, Deoxofluor or sulfur tetrafluoride. The solvent used in this step is not particularly limited in so far as it does not interfere with the reaction. Examples of the solvent include dichloromethane, tetrahydrofuran, 1,4-dioxane, 1,2-dimethoxyethane, diethyl ether, toluene, benzene, and mixed solvents thereof. The reaction temperature is preferably −78° C. to 50° C. The reaction time is not particularly limited and is usually 5 minutes to 24 hours, preferably 30 minutes to 24 hours.

When R³″″ is alkyloxy, Compound D-2 can be obtained by means of alkylation of the terminal alcohol of Compound D-1. This reaction can be performed using an appropriate base such as sodium hydride with the corresponding electrophiles such as alkyl halide, mesylate or triflate. The solvent used in this step is not particularly limited in so far as it does not interfere with the reaction. Examples of the solvent include acetone, acetonitrile, tetrahydrofuran, DMF, DMA, DMSO, toluene, and mixed solvents thereof. The reaction temperature is preferably 0° C. to 100° C. The reaction time is not particularly limited and is usually 5 minutes to 24 hours, preferably 30 minutes to 24 hours.

### General Procedure E

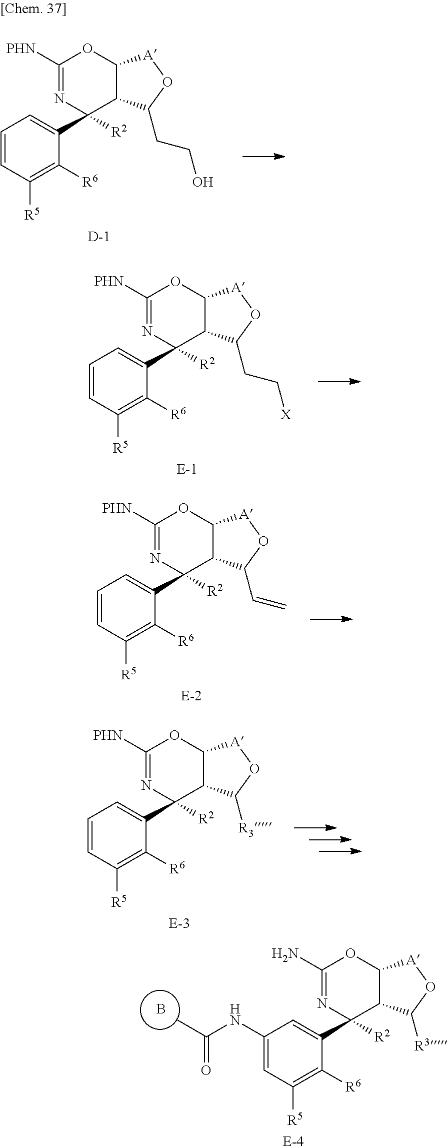

Wherein P is a protective group such as benzoyl or benzyl, R³′″″ is ethyl or cyclopropyl, X is leaving group such as halogene, mesylate or triflate, and other symbols are the same as defined above (1).

General Procedure E is a method for preparing compounds of Compound E-4 from Compound D-1 through multiple steps. Compound E-4 can be prepared from Compound E-2 according to the methods described in General procedure A. Step 1

Compound E-1 can be prepared by converting the terminal alcohol of Compound D-3 to leaving group. This reaction can be performed by a method known to a person skilled in the art. Compound E-1 can be obtained under suitable halogenation conditions such as, for example using SOX₂, POX₃ (X=Cl or Br), or Appel reaction conditions such as triphenylphosphine with CX₄ (X=Cl or Br) or iodine. The solvent used in this step is not particularly limited in so far as it does not interfere with the reaction. Examples of the solvent include dichloromethane, tetrahydrofuran, toluene, and mixed solvents thereof. The reaction temperature is preferably 0° C. to 100° C. The reaction time is not particularly limited and is usually 5 minutes to 24 hours, preferably 30 minutes to 24 hours.

### Step 2

Compound E-2 can be prepared by converting the terminal alcohol of Compound D-3 to a leaving group. This reaction can be performed by a method known to a person skilled in the art. Compound E-1 can be obtained under suitable halogenation conditions such as, for example using SOX₂, POX₃ (X=Cl or Br), or Appel reaction conditions such as triphenylphosphine with CX₄ (X=Cl or Br) or iodine. The solvent used in this step is not particularly limited in so far as it does not interfere with the reaction. Examples of the solvent include dichloromethane, tetrahydrofuran, toluene, and mixed solvents thereof. The reaction temperature is preferably 0° C. to 100° C. The reaction time is not particularly limited and is usually 5 minutes to 24 hours, preferably 30 minutes to 24 hours.

### Step 3

Compound E-2 can be prepared by means of elimination reaction of compound E-1. This reaction can be performed by a method known to a person skilled in the art. Compound E-2 can be obtained using an appropriate base such as for example, sodium or potassium tert-butoxide, triethyamine, diisopropylethylamine, DBU or pyridine. The solvent used in this step is not particularly limited in so far as it does not interfere with the reaction. Examples of the solvent include dichloromethane, tetrahydrofuran, toluene, and mixed solvents thereof. The reaction temperature is preferably 0° C. to 60° C. The reaction time is not particularly limited and is usually 5 minutes to 24 hours, preferably 30 minutes to 24 hours.

### Step 4

When R³′″″ is ethyl, Compound E-3 can be obtained by hydrogenation of Compound E-2. The hydrogenation can be performed using suitable catalysts such as, for example palladium on carbon under hydrogene atmosphere. The solvent used in this step is not particularly limited in so far as it does not interfere with the reaction. Examples of the solvent include, tetrahydrofuran, methanol, ethanol, water, mixed solvents thereof. The reaction temperature is usually room temperature to 80° C. and is preferably room temperature to 60° C. The reaction time is not particularly limited and is usually 5 minutes to 24 hours, preferably 30 minutes to 24 hours.

When R³′″″ is cyclopropyl, Compound E-3 can be obtained by means of cyclopropanation of Compound C-4. This type of reaction can be performed using an appropriate reagent such as diazomethane with or without a suitable catalyst, or Simmons-Smith reaction conditions such as, for example diiodomethane with diethylzinc. The solvent used in this step is not particularly limited in so far as it does not interfere with the reaction. Examples of the solvent include dichloromethane, diethylether, toluene, benzene, or mixed solvents thereof. The reaction temperature is usually −30° C. to room temperature. The reaction time is not particularly limited and is usually 5 minutes to 24 hours, preferably 30 minutes to 24 hours.

General Procedure F

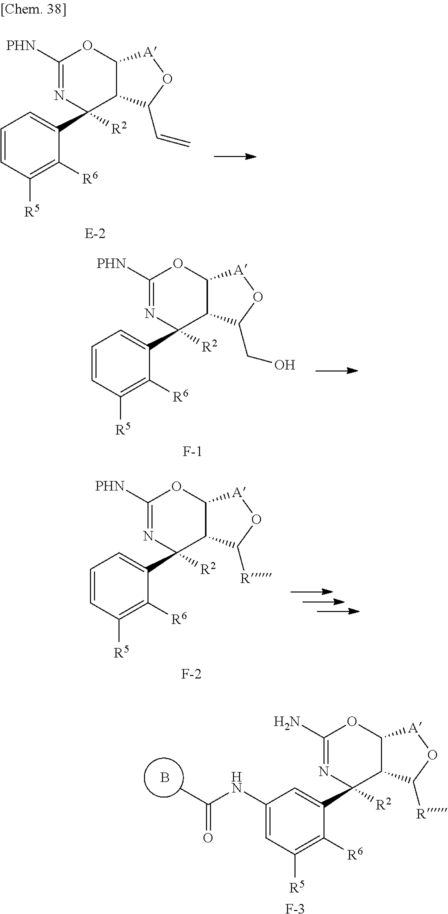

Wherein P is a protective group such as benzoyl or benzyl, R³″″″ is alkyl substituted with fluorine or alkyloxy, and other symbols are the same as defined above.

General Procedure F is a method for preparing Compound F-3 from Compounds E2 through multiple steps. Compounds F-3 can be prepared from Compound F-2 according to the methods described in General procedure A.

### Step 1

Compound F-1 can be prepared by ozonolysis of Compound F-3, followed by reduction of the resulting aldehyde. This reaction can be performed by a method known to a person skilled in the art. The ozonolysis can be performed under ozone atmosphere in a suitable solvent such as dichloromethane, methanol, and mixed thereof, with appropriate reagents such as triphenylphosphine, pyridine, dimethylsulfide and trimethylamine under nitrogene atmosphere for reductive workup. The temperature for generation of ozonide is preferably −78° C., then the temperature can be allowed to warm to room temperature for reductive workup. The reaction time is not particularly limited and is usually 30 minutes to 5 hours, preferably 30 minutes to 2 hours. The reduction of the resulting aldehyde can be performed in one pot using an appropriate reducing agent such as sodium borohydride or lithium aluminum hydride. The reaction temperature is preferably 0° C. to room temperature. The reaction time is not particularly limited and is usually 30 minutes to 5 hours, preferably 30 minutes to 2 hours.

### Step 2

When R³″″″ is CF₃, CHF₂ or CH₂F, Compound F-2 can be obtained by two-step sequence; oxidation of Compound F-1 to the aldehyde or carboxylic acid followed by fluorination, or direct fluorination of Compound F-1. This reaction can be performed by a method known to a person skilled in the art. For example, Compound F-1 can be oxidized to the corresponding aldehyde under an appropriate oxidation condition such as, for example TEMPO, Dess-Martin or Swern oxidation. The corresponding carboxylic acid can be obtained by oxidation of the resulting aldehyde, or oxidizing Compound F-1 directly using an appropriate condition such as for example, Pinnick, TEMPO or Jonse oxadation. The solvent used in this step is not particularly limited in so far as it does not interfere with the reaction. The reaction temperature is usually −78° C. to room temperature. The reaction time is not particularly limited and is usually 5 minutes to 24 hours, preferably 30 minutes to 24 hours. The flurorination reaction can be performed using an appropriate reagent such as, for example DAST, Deoxofluor or sulfur tetrafluoride. The solvent used in this step is not particularly limited in so far as it does not interfere with the reaction. Examples of the solvent include dichloromethane, tetrahydrofuran, 1,4-dioxane, 1,2-dimethoxyethane, diethyl ether, toluene, benzene, and mixed solvents thereof. The reaction temperature is preferably −78° C. to 50° C. The reaction time is not particularly limited and is usually 5 minutes to 24 hours, preferably 30 minutes to 24 hours.

When R³″″″ is alkyloxy, Compound F-2 can be obtained by means of alkylation of the terminal alcohol of Compound F-1. This reaction can be performed using an appropriate base such as sodium hydride with the corresponding electrophiles such as alkyl halide, mesylate or triflate. The solvent used in this step is not particularly limited in so far as it does not interfere with the reaction. Examples of the solvent include acetone, acetonitrile, tetrahydrofuran, DMF, DMA, DMSO, toluene, and mixed solvents thereof. The reaction temperature is preferably 0° C. to 100° C. The reaction time is not particularly limited and is usually 5 minutes to 24 hours, preferably 30 minutes to 24 hours.

The compounds of the present invention have BACE1 inhibitory activity and are effective in treatment and/or prevention, symptom improvement, and prevention of the progression of disease induced by the production, secretion or deposition of—amyloid β protein, such as Alzheimer's disease, Alzheimer dementia, senile dementia of Alzheimer type, mild cognitive impairment (MCI), prodromal Alzheimer's disease (e.g., MCI due to Alzheimer's disease), Down's syndrome, memory impairment, prion disease (Creutzfeldt-Jakob disease), Dutch type of hereditary cerebral hemorrhage with amyloidosis, cerebral amyloid angiopathy, other type of degenerative dementia, mixed dementia such as coexist Alzheimer's disease with vascular type dementia, dementia with Parkinson's Disease, dementia with progressive supranuclear palsy, dementia with Cortico-basal degeneration, Alzheimer's disease with diffuse Lewy body disease, age-related macular degeneration, Parkinson's Disease, amyloid angiopathy or the like.

Furthermore, the compounds of the present invention are effective in preventing the progression in a patient asymptomatic at risk for Alzheimer dementia (preclinical Alzheimer's disease).

“A patient asymptomatic at risk for Alzheimer dementia” includes a subject who is cognitively and functionally normal but has potential very early signs of Alzheimer's disease or typical age related changes (e.g., mild white matter hyper intensity on MRI), and/or have evidence of amyloid deposition as demonstrated by low cerebrospinal fluid Aβ₁₋₄₂ levels. For example, “a patient asymptomatic at risk for Alzheimer dementia” includes a subject whose score of the Clinical Dementia Rating (CDR) or Clinical Dementia Rating-Japanese version (CDR-J) is 0, and/or whose stage of the Functional Assessment Staging (FAST) is stage 1 or stage 2.

The compound of the present invention has not only BACE1 inhibitory activity but the beneficialness as a medicament. The compound has, preferably, any one or more of the following superior properties.

a) The compound has weak inhibitory activity for CYP enzymes such as CYP1A₂, CYP2C9, CYP2C19, CYP2D6, CYP3A₄.
b) The compound shows excellent pharmacokinetics profiles such as high bioavailability or low clearance.
c) The compound has a high metabolic stability.
d) The compound does not show irreversible inhibitions to CYP enzymes such as CYP3A₄ in the range of the concentrations of the measurement conditions described in this description.
e) The compound does not show a mutagenesis.
f) The compound is at a low risk for cardiovascular systems.
g) The compound shows a high solubility.
h) The compound shows a high brain distribution.
i) The compound has a high oral absorption.
j) The compound has a long half-life period.
k) The compound has a high protein unbinding ratio.
l) The compound is negative in the Ames test.
m) The compound has a high BACE1 selectivity over BACE2.
n) The compound has a weak mechanism based inhibition against CYP enzymes. For example, the reactive metabolites of the compound have week inhibition against CYP enzymes.
o) The compound generates little reactive metabolites.
p) The compound is a weak P-gp substrate.

Since the compound of the present invention has high inhibitory activity on BACE1 and/or high selectivity on other enzymes, for example, BACE2, it can be a medicament with reduced side effect. Further, since the compound has high effect of reducing amyloid β production in a cell system, particularly, has high effect of reducing amyloid β production in brain, it can be an excellent medicament. In addition, by converting the compound into an optically active compound having suitable stereochemistry, the compound can be a medicament having a wider safety margin on the side effect.

When a pharmaceutical composition of the present invention is administered, it can be administered orally or parenterally. The composition for oral administration can be administered in usual dosage forms such asoral solid formulations (e.g., tablets, powders, granules, capsules, pills, films or the like), oral liquid formulations (e.g., suspension, emulsion, elixir, syrup, lemonade, spirit, aromatic water, extract, decoction, tincture or the like) and the like may prepared according to the usual method and administered. The tablets can be sugar-coated tablets, film-coated tablets, enteric-coating tablets, sustained-release tablets, troche tablets, sublingual tablets, buccal tablets, chewable tablets or orally disintegrated tablets. Powders and granules can be dry syrups. Capsules can be soft capsules, micro capsules or sustained-release capsules.

The composition for parenteral administration can be administered suitably in usual parenteral dosage forms such as dermal, subcutaneous, intravenous, intraarterial, intramuscular, intraperitoneal, transmucosal, inhalation, transnasal, ophthalmic, inner ear or vaginal administration and the like. In case of parenteral administration, any forms, which are usually used, such as injections, drips, external preparations (e.g., ophthalmic drops, nasal drops, ear drops, aerosols, inhalations, lotion, infusion, liniment, mouthwash, enema, ointment, plaster, jelly, cream, patch, cataplasm, external powder, suppository or the like) and the like can be preferably administered. Injections can be emulsions whose type is O/W, W/O, O/W/O, W/O/W or the like.

The compounds of the present invention can be preferably administered in an oral dosage form because of their high oral absorbability.

A pharmaceutical composition can be formulated by mixing various additive agents for medicaments, if needed, such as excipients, binders, disintegrating agents, and lubricants which are suitable for the formulations with an effective amount of the compound of the present invention. Furthermore, the pharmaceutical composition can be for pediatric patients, geriatric patients, serious cases or operations by appropriately changing the effective amount of the compound of the present invention, formulation and/or various pharmaceutical additives. The pediatric pharmaceutical compositions are preferably administered to patients under 12 or 15 years old. In addition, the pediatric pharmaceutical compositions can be administered to patients who are under 27 days old after the birth, 28 days to 23 months old after the birth, 2 to 11 years old, 12 to 16 years old, 17 years old or 18 years old. The geriatric pharmaceutical compositions are preferably administered to patients who are 65 years old or over.

The dosage of a pharmaceutical composition of the present invention should be determined in consideration of the patient's age and body weight, the type and degree of diseases, the administration route and the like. The usual oral dosage for adults is in the range of 0.05 to 100 mg/kg/day and preferable is 0.1 to 10 mg/kg/day. For parenteral administration, the dosage highly varies with administration routes and the usual dosage is in the range of 0.005 to 10 mg/kg/day and preferably 0.01 to 1 mg/kg/day. The dosage may be administered once or several times per day.

The compound of the present invention can be used in combination with other drugs for treating Alzheimer's disease, Alzheimer dementia or the like such as acetylcholinesterase inhibitor (hereinafter referred to as a concomitant medicament) for the purpose of enforcement of the activity of the compound or reduction of the amount of medication of the compound or the like. In this case, timing of administration of the compound of the present invention and the concomitant medicament is not limited and these may be administered to the subject simultaneously or at regular intervals. Furthermore, the compound of the present invention and concomitant medicament may be administered as two different compositions containing each active ingredient or as a single composition containing both active ingredient.

The dose of the concomitant medicament can be suitably selected on the basis of the dose used on clinical. Moreover, the mix ratio of the compound of the present invention and a concomitant medicament can be suitably selected in consideration of the subject of administration, administration route, target diseases, symptoms, combinations, etc. For example, when the subject of administration is human, the concomitant medicament can be used in the range of 0.01 to 100 parts by weight relative to 1 part by weight of the compounds of the present invention.

Examples of a concomitant medicament are Donepezil hydrochloride, Tacrine, Galanthamine, Rivastigmine, Zanapezil, Memantine and Vinpocetine.

### EXAMPLE

Following examples and test examples illustrate the present invention in more detail, but the present invention is not limited by these examples.

In examples, the meaning of each abbreviation is as follows:

### Ac: Acetyl

Et: ethyl
Bz: benzoyl
DCC: dicyclohexylcarbodiimide
DIC: diisopropylcarbodiimide
iPr: isopropyl
Me: methyl
Ph: phenyl
t-Bu: tert-butyl
TBS: tert-butyldimethylsilyl
AIBN: azobisisobutyronitrile

### ADDP: 1,1′-(Azodicarbonyl)dipiperidine

AIBN: 2,2′-azobis(isobutyronitrile)
(Boc)₂O: di-tert-butyl Dicarbonate
BOMCl: benzyl chloromethyl etheroxymethyl chloride
DAST: N,N-diethylaminosulfur trifluoride
DBU: 1,8-Diazabicyclo[5.4.0]-7-undecene
DCM: dichloromethane
DEAD: diethyl azodicarboxylate
DIAD: diisopropyl azodicarboxylate
DIBAL: diisobutylaluminum Hydride

### DIPEA: N,N-diisopropylethylamine

### DMA: N,N-dimethylacetamide

DMAP: 4-dimethylaminopyridine

### DMF: N,N-dimethylformamide

DMSO: dimethylsulfoxide
EDC: 1-ethyl-3-(3-dimethylaminopropyl)carbodiimide
HATU: O-(7-aza-1H-benzotriazol-1-yl)-N,N,N′,N-tetramethyluronium hexafluorophosphate
HOAt: 1-hydroxy-7-aza-benzotriazole
HOBt: 1-hydroxy-benzotriazole
LDA: lithium diisopropylamide
LHMDS: lithium bis(trimethylsilyl)amide
mCPBA: m-chloroperoxybenzoic acid

### NCS: N-chlorosuccinimide

### NMP: N-methylpyrrolidone

PPTS: pyridinium p-toluenesulfonate
PyBOP: 1H-Benzotriazol-1-yloxy-tri(pyrrolidino) phosphonium hexafluorophosphate
TEMPO: 2,2,6,6-tetramethylpiperidine 1-oxyl free radical
TFA: trifluoroacetic acid
THF: tetrahydrofuran
THP: 2-tetrahydropyranyl

¹H NMR spectra were recorded on Bruker Advance 400 MHz spectrometer with chemical shift reported relative to tetramethylsilane or the residual solvent peak (CDCl₃=7.26 ppm, DMSO-d₆=2.50 ppm).

Analytical LC/MS (ESI positive or negative, retention time (RT)) data were recorded on Shimadzu UFLC or Waters UPLC system under the following conditions:

### Method A

Column: XBridge (Registered trademark) C18 (5 μm, i.d.4.6×50 mm) (Waters) Flow rate: 3 mL/min
UV detection wavelength: 254 nm
Mobile phase: [A] is 0.1% formic acid solution, and [B] is 0.1% formic acid in acetonitrile solvent.
Gradient: linear gradient of 10% to 100% solvent [B] for 3 minutes was performed, and 100% solvent [B] was maintained for 1 minute.

### Method B

Column: Shim-pack XR-ODS (2.2 μm, i.d. 50×3.0 mm) (Shimadzu)
Flow rate: 1.6 mL/min
Column oven: 50° C.
UV detection wavelength: 254 nm
Mobile phase: [A] 0.1% formic acid-containing aqueous solution; [B] 0.1% formic acid-containing acetonitrile solution
Gradient: linear gradient from 10% to 100% solvent [B] for 3 minutes and 100% solvent [B] for 1 minute

### Method C

Column: BEH C18 (1.7 μm, 2.1×50 mm) (Waters)
Flow rate: 0.8 mL/min
UV detection wavelength: 254 nm
Mobile phase: [A] is 10 mM CH₃COONH₄ in 95% H₂O+5% CH₃CN, and [B] is acetonitrile.
Gradient: linear gradient of 5% to 95% solvent [B] for 1.3 minutes was performed, and 95% solvent [B] was maintained for 0.7 minutes.

### Method D

Column: HSS T3 (1.8 μm, 2.1×100 mm) (Waters)
Flow rate: 0.7 mL/min
UV detection wavelength: 254 nm
Mobile phase: [A] is 10 mM CH₃COONH₄ in 95% H₂O+5% CH₃CN, and [B] is acetonitrile.
Gradient: linear gradient of 0% to 95% solvent [B] for 2.1 minutes was performed, and 95% solvent [B] was maintained for 0.5 minutes.

### Example 1

### Synthesis of Compound I-001

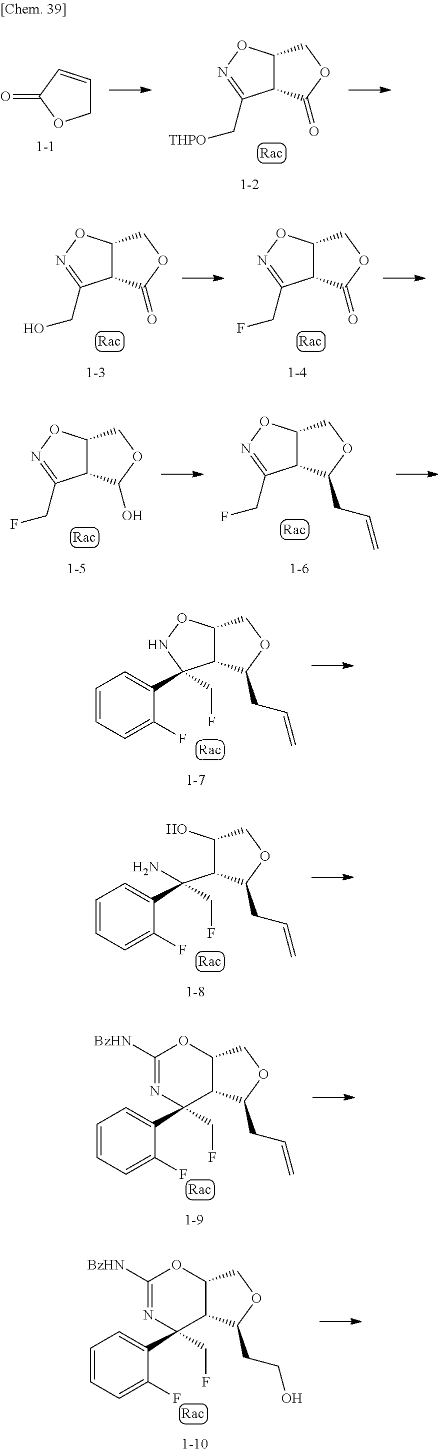

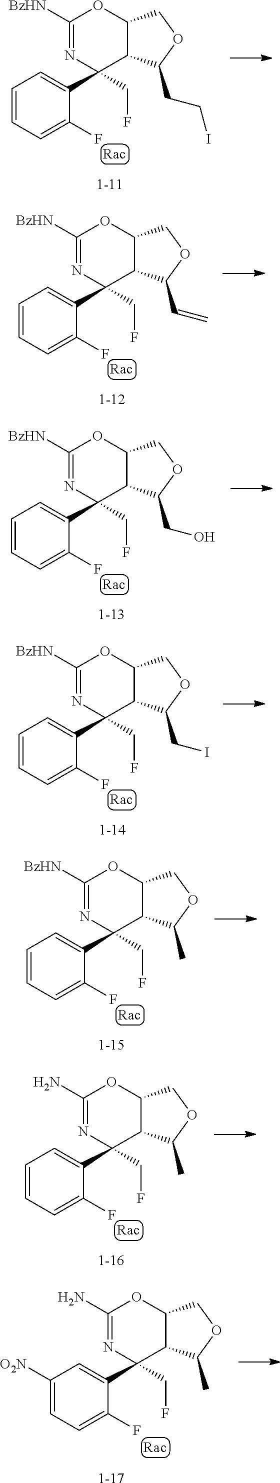

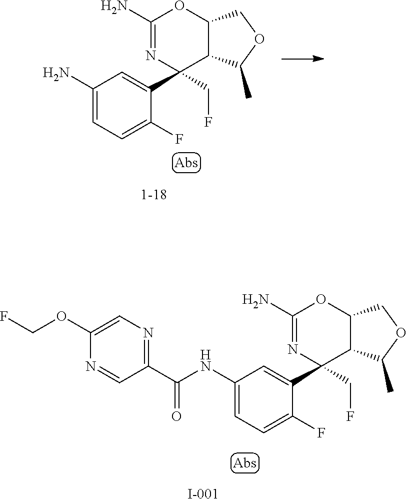 [SMILES: C[C@@H]1OC[C@H]2OC(N)=N[C@](CF)(c3cc(N)ccc3F)[C@@H]12 ; "1-18" ; C[C@@H]1OC[C@H]2OC(N)=N[C@](CF)(c3cc(NC(=O)c4cnc(OCF)cn4)ccc3F)[C@@H]12 ; "I-001"]

### Step 1: Synthesis of Compound 1-2

To a solution of gamma-Crotonolactone 1-1 (4.80 g, 57.1 mmol) and phenyl isocyanate (12.4 ml, 114 mmol) in toluene (72 ml) were added 2-(2-nitroethoxy)tetrahydro-2H-pyran (15.0 g, 86.0 mmol) and DIPEA (0.499 ml, 2.85 mmol) in toluene (24 ml) at 110° C. After stirring for 3 hours at reflux temperature, the reaction mixture was added to DIPEA (0.499 ml, 2.85 mmol). After stirring for 1 h at reflux temperature, the reaction mixture was cooled to room temperature. The mixture was filtered through Celite (Registered trademark) pad and the filtrate was concentrated in vacuo. The crude product was added to a silica gel column and eluted with hexane/EtOAc 20% to 50%. Collected fractions were evaporated to afford Compound 1-2 (6.50 g, 26.9 mmol, 47%) as a brown oil.

¹H NMR (CDCl₃) δ: 1.57-1.90 (6H, m), 3.51-3.62 (1H, m), 3.90 (1H, dt, J=38.1, 9.9 Hz), 4.33-4.48 (2H, m), 4.54-4.79 (4H, m), 5.56-5.49 (1H, m).

### Step 2: Synthesis of Compound 1-3

To a solution of Compound 1-2 (6.50 g, 26.9 mmol) in MeOH (65 ml) was added TsOH-H₂O (0.513 g, 2.69 mmol) at room temperature. After stirring for 2 hours at the same temperature, Et₃N (0.373 ml, 2.69 mmol) was added to the reaction mixture, and then concentrated in vacuo. The crude product was added to a silica gel column and eluted with hexane/EtOAc 30% to 100%. Collected fractions were evaporated to afford compound 1-3 (2.78 g, 17.7 mmol, 66%) as a brown oil.

¹H NMR (CDCl₃) δ: 2.38 (1H, br s), 4.44 (1H, d, J=9.7 Hz), 4.74-4.58 (4H, m), 5.57-5.52 (1H, m).

### Step 3: Synthesis of Compound 1-4

To a solution of compound 1-3 (2.78 g, 17.7 mmol) in CH₂Cl₂ (28 ml) was added 90% DAST (3.12 ml, 21.2 mmol) at −78° C. The reaction mixture was stirred for 5 hours at room temperature and was treated with aqueous potassium carbonate. The mixture was extracted with EtOAc, and the organic layer was washed with water. The organic layer was concentrated in vacuo. The crude product was added to a silica gel column and eluted with hexane/EtOAc 30%. Collected fractions were evaporated to afford compound 1-4 (1.58 g, 9.93 mmol, 56%) as a brown oil.

¹H NMR (CDCl₃) δ: 4.41 (1H, d, J=9.7 Hz), 4.62 (1H, d, J=11.3 Hz), 4.69 (1H, dd, J=11.3, 5.3 Hz), 5.20-5.41 (2H, m), 5.63-5.57 (1H, m).

### Step 4: Synthesis of Compound 1-5

To a solution of Compound 1-4 (1.58 g, 9.93 mmol) in CH₂Cl₂ (12 ml) and toluene (24 ml) was added to DIBAL (1.02 M in hexane, 10.7 ml, 10.9 mmol) at −78° C. After stirring for 30 min at the same temperature, the reaction mixture was added to MeOH (1.33 ml, 32.8 mmol), THF (24 ml) and H₂O (0.885 ml, 49.2 mmol). After stirring for 30 min at room temperature, the mixture was filtered through Celite (Registered trademark) pad and the filtrate was concentrated in vacuo. The crude product was added to a silica gel column and eluted with hexane/EtOAc 30%. Collected fractions were evaporated to afford Compound 1-5 (829 mg, 5.14 mmol, 52%) as a yellow solid.

¹H NMR (CDCl₃) δ: 2.60 (1H, d, J=2.3 Hz), 3.95 (1H, d, J=8.9 Hz), 4.25-4.31 (2H, m), 5.15 (1H, dd, J=18.4, 11.7 Hz), 5.26 (1H, dd, J=18.4, 11.7 Hz), 5.40-5.36 (1H, m), 5.72 (1H, d, J=2.1 Hz).

### Step 5: Synthesis of Compound 1-6

To a solution of Compound 1-5 (770 mg, 4.78 mmol) and allyltrimethylsilane (3.80 ml, 23.9 mmol) in DCM (15 ml) and MeCN (15 ml) was added BF₃-OEt₂ (3.03 ml, 23.9 mmol) at 0° C. After being stirred for 1 hour at room temperature, the reaction was quenched with aqueous sodium carbonate solution. The mixture was extracted with ethyl acetate and the combined organic layers were washed with water. The organic layer was concentrated in vacuo. The crude product was added to a silica gel column and eluted with hexane/EtOAc 30%. Collected fractions were evaporated to afford Compound 1-6 (711 mg, 3.84 mmol, 80%) as a colorless oil.

¹H NMR (CDCl₃) δ: 2.25-2.44 (2H, m), 3.74 (1H, d, J=9.5 Hz), 4.03-4.14 (2H, m), 4.24-4.29 (1H, m), 5.08-5.26 (4H, m), 5.35-5.29 (1H, m), 5.86-5.75 (1H, m).

### Step 6: Synthesis of Compound 1-7

To a solution of 1-bromo-2-fluorobenzene (1.68 g, 9.60 mmol) in toluene (28 mL) and THF (7 mL) was added n-BuLi (1.64 M in n-hexane, 5.85 mL, 9.60 mmol) at −78° C. and stirred for 10 minutes at the same temperature. To the reaction mixture were added BF₃-OEt₂ (0.487 ml, 3.84 mmol) and a solution of Compound 1-6 in toluene (7 mL) at −78° C. and stirred for 1 hour at the same temperature. To the reaction mixture was added aqueous NH₄Cl solution and the aqueous layer was extracted with EtOAc. The organic layer was washed with water and concentrated in vacuo. The crude product was added to a silica gel column and eluted with hexane/EtOAc 30%. Collected fractions were evaporated to afford Compound 1-7 (883 mg, 3.14 mmol, 82%) as a colorless oil.

¹H NMR (CDCl₃) δ: 2.34 (2H, t, J=6.7 Hz), 3.23-3.29 (1H, m), 3.96 (2H, d, J=3.3 Hz), 4.52-4.76 (3H, m), 4.95 (1H, dd, J=10.4, 47.1 Hz), 5.14 (1H, s), 5.18 (1H, d, J=5.1 Hz), 5.80-5.93 (1H, m), 6.18 (1H, brs), 7.03-7.12 (1H, m), 7.15-7.20 (1H, m), 7.28-7.34 (1H, m), 7.69 (1H, brs).

### Step 7: Synthesis of Compound 1-8

To a solution of Compound 1-7 (883 mg, 3.14 mmol) in AcOH (8.8 ml) was added Zn (2.05 g, 31.4 mmol) at room temperature. After stirring for 1 hour at 60° C., the reaction mixture was cooled to room temperature, and aqueous potassium carbonate was added to this mixture. The mixture was filtered through Celite (Registered trademark) pad and the filtrate was extracted with EtOAc. The organic layer was washed with water and concentrated in vacuo. The crude product was added to a silica gel column and eluted with Hexane/EtOAc 30% to 100%. Collected fractions were evaporated to afford Compound 1-8 (783 mg, 2.76 mmol, 88%) as a colorless oil.

¹H NMR (CDCl₃) δ: 2.21-2.31 (1H, m), 2.50-2.58 (1H, m), 2.65 (1H, dd, J=8.5, 4.6 Hz), 3.63-3.73 (2H, m), 3.96 (1H, t, J=3.3 Hz), 4.34-4.40 (1H, m), 4.50 (1H, dd, J=47.8, 9.3 Hz), 4.90 (1H, ddd, J=47.8, 9.3, 2.8 Hz), 5.12 (1H, s), 5.15 (1H, s), 5.84-5.96 (1H, m), 7.09 (1H, dd, J=12.7, 8.2 Hz), 7.15-7.20 (1H, m), 7.21-7.28 (1H, m), 7.32-7.38 (1H, m), 7.62-7.68 (1H, m).

### Step 8: Synthesis of Compound 1-9

To a solution of Compound 1-8 (783 mg, 2.76 mmol) in CH₂Cl₂ (7.8 ml) was added benzoyl isothiocyanate (0.417 ml, 3.04 mmol) at room temperature. After stirring for 1 day at the same temperature, the reaction mixture was added to EDC-HCl (1.06 g, 5.53 mmol). After stirring for 1 day at the same temperature, the reaction mixture was concentrated in vacuo. The crude product was added to a silica gel column and eluted with Hexane/EtOAc 10% to 50%. Collected fractions were evaporated to afford Compound 1-9 (864 mg, 2.10 mmol, 76%) as a white amorphous.

¹H NMR (CDCl₃) δ: 2.31-2.40 (1H, m), 2.65-2.73 (1H, m), 3.15 (1H, dd, J=8.8, 4.2 Hz), 3.86 (1H, dd, J=10.7, 2.5 Hz), 4.19 (1H, d, J=10.7 Hz), 4.41-4.47 (1H, m), 4.57-4.61 (1H, m), 4.85 (2H, dt, J=9.5, 46.6 Hz), 5.22-5.29 (2H, m), 5.87-5.98 (1H, m), 7.14-7.25 (2H, m), 7.33-7.55 (5H, m), 8.27 (2H, d, J=7.4 Hz), 12.16 (1H, brs).

### Step 9: Synthesis of Compound 1-10

A solution of Compound 1-9 (864 mg, 2.10 mmol) in CH₂Cl₂ (17 ml) was stirred under ozone atmosphere at −78° C. After stirring for 20 minutes at the same temperature, to the reaction mixture was added PPh₃ (1.26 g, 4.82 mmol) under Na atmosphere. After stirring for 1.5 hours at room temperature, the reaction mixture was added MeOH (8.6 ml) and NaBH₄ (238 mg, 6.28 mmol). After stirring for 2 h at the same temperature, to the reaction mixture was added aqueous NH₄Cl solution, and the aqueous layer was extracted with EtOAc. The organic layer was washed with water and was concentrated in vacuo. The crude product was added to a silica gel column and eluted with hexane/EtOAc 30% to 100%. Collected fractions were evaporated to afford Compound 1-10 (872 mg, 2.10 mmol, 100%) as a white amorphous.

¹H NMR (CDCl₃) δ: 1.87-1.97 (1H, m), 1.99-2.08 (1H, m), 2.29-2.34 (1H, m), 3.11 (1H, dd, J=9.1, 4.2 Hz), 3.83-3.97 (3H, m), 4.21 (1H, d, J=10.5 Hz), 4.51 (1H, t, J=8.8 Hz), 4.58-4.62 (1H, m), 4.86 (2H, ddd, J=46.7, 20.7, 9.3 Hz), 7.14-7.25 (2H, m), 7.35-7.51 (5H, m), 8.27 (2H, d, J=7.3 Hz), 12.18 (1H, brs).

### Step 10: Synthesis of Compound 1-11

To a solution of Compound 1-10 (872 mg, 2.10 mmol), PPh₃ (1.10 g, 4.19 mmol) and imidazole (285 mg, 4.19 mmol) in THF (17 ml) was added 12 (1.06 g, 4.19 mmol) at 0° C. After stirring for 1.5 hours at the same temperature, to the reaction mixture was added aqueous NaHSO₃ solution, and the aqueous layer was extracted with EtOAc. The organic layer was washed with water and was concentrated in vacuo. The crude product was added to a silica gel column and eluted with hexane/EtOAc 10% to 50%. Collected fractions were evaporated to afford Compound 1-11 (911 mg, 1.73 mmol, 83%) as a white amorphous.

¹H NMR (CDCl₃) δ: 2.12-2.29 (2H, m), 3.04 (1H, dd, J=8.7, 4.4 Hz), 3.29 (1H, q, J=8.7 Hz), 3.38-3.44 (1H, m), 3.86 (1H, dd, J=10.8, 2.2 Hz), 4.20 (1H, d, J=10.8 Hz), 4.37 (1H, t, J=8.7 Hz), 4.59-4.63 (1H, m), 4.85 (2H, ddd, J=46.7, 22.6, 9.5 Hz), 7.13-7.26 (2H, m), 7.33-7.56 (5H, m), 8.28 (2H, d, J=7.5 Hz), 12.18 (1H, brs).

### Step 11: Synthesis of Compound 1-12

To a solution of KO^{t}Bu (1.0 M in THF, 6.92 ml, 6.92 mmol) in THF (9 ml) was added Compound 1-11 (911 mg, 1.73 mmol) in THF (9 ml) at 0° C. After stirring for 30 min at the same temperature, the reaction mixture was treated with aqueous NH₄Cl solution, and the aqueous layer was extracted with AcOEt. The combined organic layers were washed with H₂O and brine, dried over Na₂SO₄ and filtered. The filtrate was concentrated under vacuum to give Compound 1-12 (677 mg, 1.70 mmol, 98%) as a white solid that was used for the next step without purification.

¹H NMR (CDCl₃) δ: 3.07 (1H, dd, J=9.6, 4.1 Hz), 4.02 (1H, dd, J=10.7, 2.9 Hz), 4.24 (1H, d, J=10.7 Hz), 4.61-5.00 (4H, m), 5.42 (1H, d, J=10.2 Hz), 5.54 (1H, d, J=16.9 Hz), 5.91-6.01 (1H, m), 7.13-7.25 (2H, m), 7.34-7.56 (5H, m), 8.29 (2H, d, J=7.5 Hz), 12.23 (1H, brs).

### Step 12: Synthesis of Compound 1-13

A solution of Compound 1-12 (452 mg, 1.14 mmol) in CH₂Cl₂ (23 ml) was stirred under ozone atmosphere at −78° C. After stirring for 20 minutes at the same temperature, to the reaction mixture was added PPh₃ (684 mg, 2.61 mmol) under N₂ atmosphere. After stirring for 1.5 hours at room temperature, to the reaction mixture were added MeOH (11 ml) and NaBH₄ (129 mg, 3.40 mmol). After stirring for 1.5 hours at the same temperature, to the reaction mixture was added aqueous NH₄Cl solution, and the aqueous layer was extracted with EtOAc. The organic layer was washed with water and was concentrated in vacuo. The crude product was added to a silica gel column and eluted with hexane/EtOAc 30% to 100%. Collected fractions were evaporated to afford Compound 1-13 (457 mg, 1.14 mmol, 100%) as a white amorphous.

¹H NMR (CDCl₃) δ: 1.98 (1H, dd, J=5.4, 7.8 Hz), 3.50 (1H, dd, J=8.9, 4.3 Hz), 3.70-3.78 (1H, m), 3.92 (1H, dd, J=10.5, 2.4 Hz), 3.99-4.05 (1H, m), 4.24 (1H, d, J=10.5 Hz), 4.46-4.40 (1H, m), 4.65 (1H, t, J=3.2 Hz), 4.86 (2H, ddd, J=10.2, 12.5, 47.2 Hz), 7.14-7.25 (2H, m), 7.34-7.52 (5H, m), 8.27 (2H, d, J=7.4 Hz), 12.17 (1H, brs).

### Step 13: Synthesis of Compound 1-14

To a solution of Compound 1-13 (457 mg, 1.14 mmol), PPh₃ (596 mg, 2.27 mmol) and imidazole (155 mg, 2.27 mmol) in THF (9 ml) was added 12 (576 mg, 2.27 mmol) at 0° C. After stirring for 1.5 hours at room temperature, to the reaction mixture was added aqueous NaHSO₃ and the aqueous layer was extracted with EtOAc. The organic layer was washed with water and concentrated in vacuo. The crude product was added to a silica gel column and eluted with hexane/EtOAc 10% to 50%. Collected fractions were evaporated to afford Compound 1-14 (418 mg, 0.816 mmol, 72%) as a white amorphous.

¹H NMR (CDCl₃) δ: 3.42 (1H, dd, J=8.6, 4.2 Hz), 3.51 (1H, dd, J=11.5, 2.8 Hz), 3.78 (1H, dd, J=11.5, 2.8 Hz), 4.06-4.16 (2H, m), 4.21 (1H, d, J=10.7 Hz), 4.62-5.01 (3H, m), 7.17-7.27 (2H, m), 7.35-7.57 (5H, m), 8.27 (2H, d, J=6.8 Hz), 12.19 (1H, brs).

### Step 14: Synthesis of Compound 1-15

To a solution of Compound 1-14 (418 mg, 0.816 mmol) in toluene (4 ml) were added Bu₃SnH (0.263 ml, 0.979 mmol) and AIBN (6.70 mg, 0.0410 mmol) at room temperature. After stirring for 1 hour at 80° C., the reaction mixture was concentrated. The resulting residue was added to an amino silica gel column and eluted with Hexane/EtOAc 10% to 50%. Collected fractions were evaporated to afford Compound 1-15 (280 mg, 0.725 mmol, 89%) as a white amorphous.

¹H NMR (CDCl₃) δ: 1.48 (3H, d, J=5.9 Hz), 2.89 (1H, dd, J=9.2, 4.1 Hz), 3.98 (1H, dd, J=10.8, 2.9 Hz), 4.17 (1H, d, J=10.8 Hz), 4.35-4.44 (1H, m), 4.61 (1H, t, J=3.5 Hz), 4.79 (1H, dd, J=10.0, 46.5 Hz), 4.94 (1H, dd, J=10.0, 46.5 Hz), 7.13-7.26 (2H, m), 7.34-7.55 (5H, m), 8.28 (2H, d, J=7.4 Hz), 12.18 (1H, brs).

### Step 15: Synthesis of Compound 1-16

To a solution of Compound 1-15 (280 mg, 0.725 mmol) in EtOH (3 ml) and THF (3 ml) was added hydrazine hydrate (0.352 ml, 7.25 mmol) at room temperature. After stirring for 14 hours at the same temperature, the reaction mixture was concentrated. The resulting residue was added to an amino silica gel column and eluted with Hexane/EtOAc 40% to 60%. Collected fractions were evaporated to afford Compound 1-16 (205 mg, 0.725 mmol, 100%) as a white amorphous.

¹H NMR (CDCl₃) δ: 1.44 (3H, d, J=5.9 Hz), 2.77 (1H, dd, J=8.9, 4.4 Hz), 3.81-3.88 (2H, m), 4.24-4.37 (3H, m), 4.54-4.76 (2H, m), 7.06 (1H, dd, J=12.5, 8.2 Hz), 7.20-7.15 (1H, m), 7.27-7.33 (1H, m), 7.42-7.47 (1H, m).

### Step 16: Synthesis of Compound 1-18

To a solution of Compound 1-16 (205 mg, 0.725 mmol) in TFA (3 ml) was added sulfuric acid (0.774 ml, 14.5 mmol) at −8° C. After stirring for 5 minutes at the same temperature, to the reaction mixture was added HNO₃ (0.0490 ml, 1.09 mmol). After stirring for 10 minutes at the same temperature, the reaction mixture was treated with aqueous K₂CO₃ solution. The aqueous layer was extracted with AcOEt, and the organic layer was dried over Na₂SO₄ and filtered. The filtrate was concentrated under vacuum to give Compound 1-17 as a white amorphous that was used for the next step without purification.

A solution of Compound 1-17 and 10% Pd—C (245 mg, 0.109 mmol) in MeOH (2 ml) was stirred under H2 atmosphere at room temperature. After stirring for 2 hours at the same temperature, the mixture was filtered through Celite (Registered trademark) pad. The filtrate was concentrated under vacuum. The resulting residue was purified by supercritical fluid chromatography (SFC) (Chiralpak (Registered trademark) IC; 40% isopropylalcohol with 0.1% diethylamine) to give Compound 1-18 (78.0 mg, 0.262 mmol, 36%).

¹H NMR (CDCl₃) δ: 1.42 (3H, d, J=6.0 Hz), 2.75 (1H, dd, J=9.0, 4.2 Hz), 3.61 (1H, brs), 3.84 (2H, dd, J=14.2, 10.4 Hz), 4.25-4.34 (1H, m), 4.34-4.37 (1H, m), 4.50-4.74 (2H, m), 6.59-6.53 (1H, m), 6.72 (1H, dd, J=6.7, 2.9 Hz), 6.85 (1H, dd, J=11.9, 8.6 Hz).

### Step 17: Synthesis of Compound I-001

To a solution of Compound 1-18 (10.0 mg, 0.0340 mmol) in MeOH (0.66 ml) were added 5-(fluoromethoxy)pyrazine-2-carboxylic acid (5.79 mg, 0.0340 mmol) and 2 mol/L HCl (0.0168 ml, 0.0340 mmol) at room temperature. To the reaction mixture was added EDC-HCl (9.86 mg, 0.0370 mmol) at the same temperature. After stirring for 30 minutes, the reaction mixture was treated with aqueous NaHCO₃. The aqueous layer was extracted with AcOEt and the organic layer was dried over Na₂SO₄, filtered and concentrated to afford Compound I-001 (10.0 mg, 0.0220 mmol, 66%) as a white solid.

¹H NMR (CDCl₃) δ: 1.45 (3H, d, J=6.0 Hz), 2.80 (1H, dd, J=8.7, 4.1 Hz), 3.87 (2H, s), 4.29-4.42 (2H, m), 4.59 (1H, dd, J=13.8, 8.7 Hz), 4.71 (1H, dd, J=13.8, 8.7 Hz), 6.15 (2H, dd, J=51.2, 5.0 Hz), 7.12 (1H, dd, J=11.6, 9.0 Hz), 7.48-7.53 (1H, m), 8.03-7.98 (1H, m), 8.30 (1H, s), 9.08 (1H, s), 9.52 (1H, s).

### Example 2

### Synthesis of Compound I-002

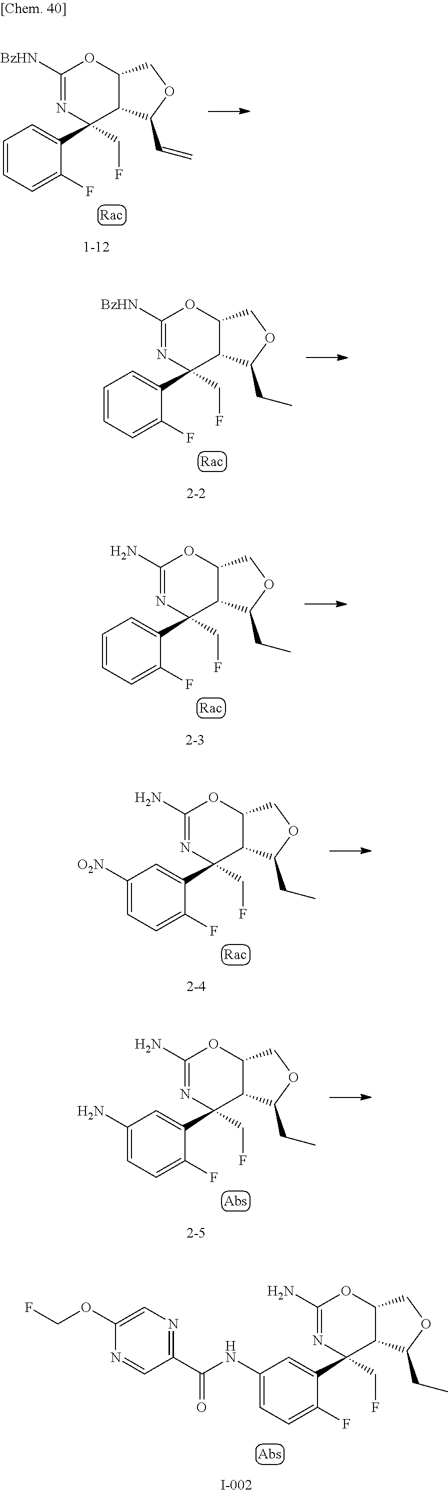

### Step 1: Synthesis of Compound 2-3

A solution of Compound 1-12 (200 mg, 0.502 mmol) and 10% Pd—C (203 mg, 0.0900 mmol) in THF (4 ml) was stirred under H2 atmosphere at room temperature. After stirring for 3 hours at the same temperature, the reaction mixture was filtered through Celite (Registered trademark) pad. The filtrate was concentrated under vacuum to give Compound 2-2 as a white amorphous that was used for the next step without purification.

To a solution of Compound 2-2 in EtOH (4 ml) was added hydrazine hydrate (0.244 ml, 5.02 mmol) at room temperature. After stirring for 30 minutes at 50° C., the reaction mixture was concentrated. The resulting residue was added to an amino silica gel column and eluted with Hexane/EtOAc 50% to 60%. Collected fractions were evaporated to afford Compound 2-3 (124 mg, 0.418 mmol, 83%) as a white amorphous.

¹H NMR (CDCl₃) δ: 1.07 (3H, t, J=7.3 Hz), 1.52-1.63 (1H, m), 1.78-1.90 (1H, m), 2.89 (1H, dd, J=8.7, 4.4 Hz), 3.73 (1H, dd, J=10.3, 2.0 Hz), 3.87 (1H, d, J=10.3 Hz), 4.14-4.20 (1H, m), 4.27-4.30 (1H, m), 4.33 (1H, brs), 4.57 (1H, dd, J=16.3, 8.9 Hz), 4.69 (1H, dd, J=16.3, 8.9 Hz), 7.06 (1H, dd, J=12.5, 7.9 Hz), 7.17 (1H, t, J=7.6 Hz), 7.27-7.33 (1H, m), 7.44 (1H, t, J=7.9 Hz).

### Step 2: Synthesis of Compound 2-5

To a solution of Compound 2-3 (124 mg, 0.418 mmol) in TFA (1.8 ml) was added sulfuric acid (0.446 ml, 8.37 mmol) at −8° C. After stirring for 5 minutes at the same temperature, to the reaction mixture was added HNO₃ (0.0280 ml, 0.628 mmol). After stirring for 10 minutes at the same temperature, the reaction mixture was treated with aqueous K₂CO₃ solution. The aqueous layer was extracted with AcOEt and the organic layer was dried over Na₂SO₄ and filtered. The filtrate was concentrated under vacuum to give Compound 2-4 as a white amorphous that was used for the next step without purification.

A solution of Compound 2-4 and 10% Pd—C (141 mg, 0.0630 mmol) in MeOH (6 ml) was stirred under H2 atmosphere at room temperature. After stirring for 2 hours at the same temperature, the reaction mixture was filtered through Celite (Registered trademark) pad. The filtrate was concentrated under vacuum. The resulting residue was purified by supercritical fluid chromatography (SFC) (Chiralpak (Registered trademark) IC; 40% isopropylalcohol with 0.1% diethylamine) to give Compound 2-5 (52.0 mg, 0.167 mmol, 40%).

¹H NMR (CDCl₃) δ: 1.06 (3H, t, J=7.3 Hz), 1.51-1.63 (1H, m), 1.77-1.89 (1H, m), 2.87 (1H, dd, J=8.7, 4.3 Hz), 3.62 (1H, brs), 3.74 (1H, dd, J=10.2, 2.2 Hz), 3.87 (1H, d, J=10.2 Hz), 4.11-4.18 (1H, m), 4.33-4.37 (1H, m), 4.54 (1H, dd, J=25.9, 8.7 Hz), 4.66 (1H, dd, J=25.9, 8.7 Hz), 6.59-6.54 (1H, m), 6.72 (1H, dd, J=6.7, 2.9 Hz), 6.84 (1H, dd, J=11.9, 8.5 Hz).

### Step 3: Synthesis of Compound I-002

To a solution of Compound 2-5 (17.0 mg, 0.0550 mmol) in MeOH (1 ml) were added 5-(fluoromethoxy)pyrazine-2-carboxylic acid (9.40 mg, 0.0550 mmol) and 2 mol/L HCl (0.0273 ml, 0.0550 mmol) at room temperature. To the reaction mixture was added EDC-HCl (16.0 mg, 0.0600 mmol) at the same temperature. After stirring for 1 hour, the reaction mixture was treated with aqueous NaHCO₃. The aqueous layer was extracted with AcOEt and the organic layer was dried over Na₂SO₄, filtered and concentrated to afford Compound I-002 (20.0 mg, 0.0430 mmol, 79%) as a white solid.

¹H NMR (CDCl₃) δ: 1.08 (3H, t, J=7.3 Hz), 1.78-1.90 (1H, m), 2.90-2.96 (1H, m), 3.76 (1H, dd, J=10.3, 2.1 Hz), 3.90 (1H, d, J=10.3 Hz), 4.17 (1H, t, J=8.5 Hz), 4.35-4.39 (1H, m), 4.63 (2H, d, J=47.1 Hz), 6.15 (2H, dd, J=51.2, 5.0 Hz), 7.11 (1H, dd, J=11.6, 8.8 Hz), 7.47-7.51 (1H, m), 7.98-8.03 (1H, m), 8.29 (1H, s), 9.08 (1H, s), 9.51 (1H, s).

### Example 3

### Synthesis of Compound I-003

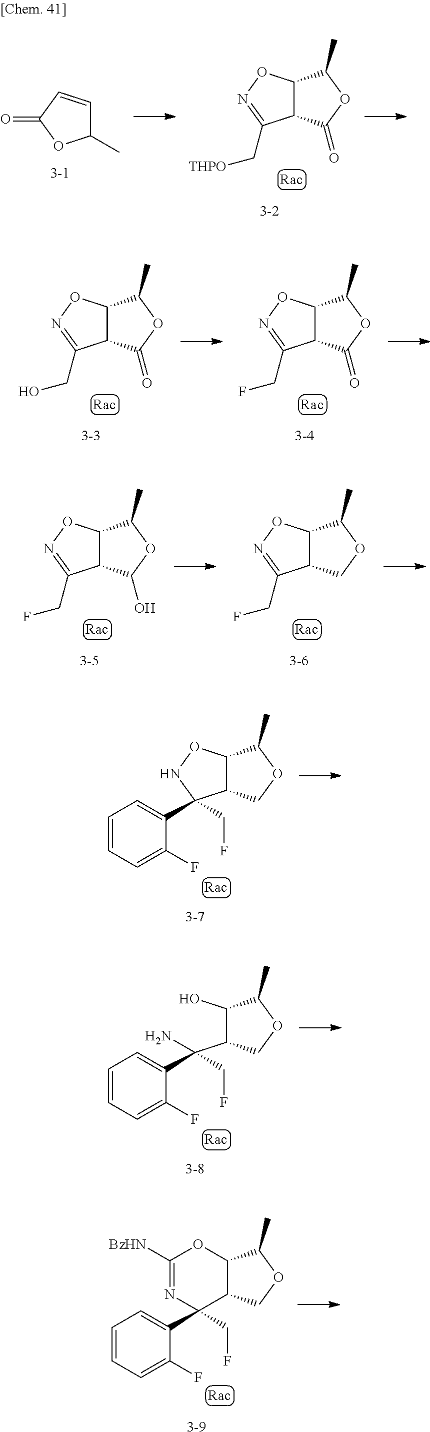

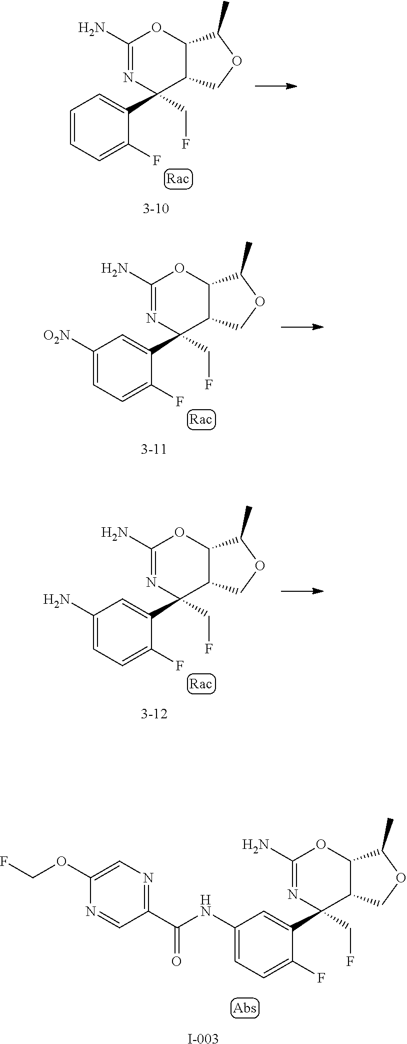

For the synthesis of I-003, racemic 3-1 was used, so compound 3-2 was a mixture of four diasteromers. Two diastereomers could be separated by silica gel chromate at step 3, so compound 3-3 was enantiomer mixture.

### Step 1: Synthesis of Compound 3-2

To a solution of 5-Methylfuran-2(5H)-one 3-1 (racemate) (10.0 g, 102 mmol) and phenyl isocyanate (22.2 ml, 204 mmol) in toluene (150 ml) were added 2-(2-nitroethoxy)tetrahydro-2H-pyran (26.8 g, 153 mmol) and DIPEA (0.890 ml, 5.10 mmol) in toluene (50 ml) at 110° C. After stirring for 3 hours at reflux temperature, the reaction mixture was cooled to room temperature. The mixture was filtered through Celite (Registered trademark) pad and the filtrate was concentrated in vacuo. The crude product was added to a silica gel column and eluted with hexane/EtOAc 20% to 50%. Collected fractions were evaporated to afford Compound 3-2 (a mixture of four diasteromers) (14.4 g, 56.4 mmol, 55%) containing impurities as a brown oil.

### Step 2: Synthesis of Compound 3-3

To a solution of Compound 3-2 (14.4 g, 56.4 mmol) in EtOH (43 ml) was added PPTS (2.84 g, 11.3 mmol) at room temperature. After stirring for 3.5 hours at 60° C., the reaction mixture was concentrated in vacuo. The crude product was added to a silica gel column and eluted with hexane/EtOAc 50% to 100%. Collected fractions were evaporated to afford Compound 3-3 (enantiomer mixture) (3.69 g, 21.6 mmol, 38%) as a brown oil.

¹H NMR (CDCl₃) δ: 1.51 (3H, d, J=6.8 Hz), 2.52-2.58 (1H, m), 4.50 (1H, d, J=9.5 Hz), 4.60-4.64 (2H, m), 4.82 (1H, dq, J=2.0, 6.8 Hz), 5.09 (1H, dd, J=9.5, 2.0 Hz).

### Step 3: Synthesis of Compound 3-4

To a solution of Compound 3-3 (3.69 g, 21.6 mmol) in CH₂Cl₂ (37 ml) was added 90% DAST (3.80 ml, 25.9 mmol) at −78° C. The reaction mixture was stirred for 2 hours at room temperature and was treated with aqueous potassium carbonate solution. The mixture was extracted with EtOAc, and the organic layer was washed with water. The organic layer was concentrated in vacuo. The crude product was added to a silica gel column and eluted with hexane/EtOAc 20% to 50%. Collected fractions were evaporated to afford Compound 3-4 (3.31 g, 19.1 mmol, 89%) as a yellow oil. ¹H NMR (CDCl₃) δ: 1.51 (3H, d, J=6.8 Hz), 4.46 (1H, dd, J=9.5, 1.6 Hz), 4.80-4.86 (1H, m), 5.16 (1H, d, J=9.5 Hz), 5.24 (1H, dd, J=19.3, 11.5 Hz), 5.36 (1H, dd, J=19.3, 11.5 Hz).

### Step 4: Synthesis of Compound 3-5

To a solution of Compound 3-4 (3.31 g, 19.1 mmol) in CH₂Cl₂ (66 ml) was added DIBAL (1.02 M in hexane, 22.5 ml, 22.9 mmol) at −78° C. After stirring for 20 minutes at the same temperature, to the reaction mixture was added aqueous Rochelle's salt. After stirring for 3 hours at room temperature, to the mixture was added 2 mol/L HCl (pH=4). The mixture was extracted with EtOAc, and the organic layer was washed with water. The organic layer was concentrated in vacuo. The crude product was added to a silica gel column and eluted with hexane/EtOAc 20% to 80%. Collected fractions were evaporated to afford Compound 3-5 (2.19 g, 12.5 mmol, 65%) containing diastereomer as a yellow solid.

### Step 5: Synthesis of Compound 3-7

To a solution of Compound 3-5 (1.96 g, 11.2 mmol) and triethylsilane (8.94 ml, 55.9 mmol) in DCM (14 ml) and MeCN (14 ml) was added BF₃-OEt₂ (7.09 ml, 55.9 mmol) at 0° C. After stirring for 15 minutes at the same temperature, the reaction mixture was treated with aqueous sodium carbonate. The aqueous layer was extracted with AcOEt and the organic layer was dried over Na₂SO₄ and filtered. The filtrate was concentrated under vacuum to give Compound 3-6 as an yellow oil that was used for the next step without purification.

To a solution of 1-bromo-2-fluorobenzene (4.90 g, 28.0 mmol) in toluene (80 mL) and THF (10 mL) was added n-BuLi (1.62 M in n-hexane, 17.1 mL, 28.0 mmol) at −78° C. and the reaction mixture was stirred for 10 minutes at the same temperature. To the reaction mixture were added BF₃-OEt₂ (1.42 ml, 11.2 mmol) and a solution of Compound 3-6 in THF (10 mL) and toluene (18 ml) at −78° C. and the reaction mixture was stirred for 30 minutes at the same temperature. To the reaction mixture was added aqueous NH₄Cl solution, and the aqueous layer was extracted with EtOAc. The organic layer was washed with water and was concentrated in vacuo. The crude product was added to a silica gel column and eluted with hexane/EtOAc 0% to 20%. Collected fractions were evaporated to afford Compound 3-7 (1.31 g, 5.13 mmol, 46%) as a yellow oil.

¹H NMR (CDCl₃) δ: 1.18 (3H, d, J=6.8 Hz), 3.52-3.59 (1H, m), 4.03 (1H, dd, J=10.6, 7.3 Hz), 4.21-4.38 (3H, m), 4.68 (1H, dd, J=47.4, 9.8 Hz), 4.85-5.03 (1H, m), 6.16 (1H, s), 7.07 (1H, dd, J=11.9, 8.2 Hz), 7.18 (1H, t, J=7.2 Hz), 7.28-7.35 (1H, m), 7.67 (1H, brs).

### Step 7: Synthesis of Compound 3-8

To a solution of Compound 3-7 (1.31 g, 5.13 mmol) in AcOH (13 ml) was added Zn (2.01 g, 30.8 mmol) at room temperature. After stirring for 2 hours at 60° C., the reaction mixture was cooled to room temperature, and to this mixture was added aqueous potassium carbonate solution. The mixture was filtered through Celite (Registered trademark) pad and the filtrate was extracted with EtOAc. The organic layer was washed with water and concentrated in vacuo. The crude product was added to a silica gel column and eluted with Hexane/EtOAc 30% to 100%. Collected fractions were evaporated to afford Compound 3-8 (1.08 g, 4.20 mmol, 82%) as a colorless oil.

¹H NMR (CDCl₃) δ: 1.06 (3H, d, J=6.8 Hz), 2.84-2.92 (1H, m), 3.55 (1H, d, J=4.3 Hz), 3.95 (1H, q, J=6.8 Hz), 4.00-4.06 (1H, m), 4.09-4.19 (1H, m), 4.34 (1H, dd, J=48.2, 9.1 Hz), 4.90 (1H, ddd, J=48.2, 9.1, 2.5 Hz), 7.08-7.15 (1H, m), 7.23-7.27 (1H, m), 7.33-7.40 (1H, m), 7.70-7.64 (1H, m).

Step 8: Synthesis of compound 3-9

To a solution of Compound 3-8 (1.08 g, 4.20 mmol) in CH₂Cl₂ (11 ml) was added benzoyl isothiocyanate (0.633 ml, 4.62 mmol) at room temperature. After stirring for 3 hours at the same temperature, to the reaction mixture was added EDC-HCl (1.61 g, 8.40 mmol). After stirring for 14 hours at the same temperature, the reaction mixture was concentrated in vacuo. The crude product was added to a silica gel column and eluted with Hexane/EtOAc 10% to 50%. Collected fractions were evaporated to afford Compound 3-9 (1.04 g, 2.69 mmol, 64%) as a white solid.

¹H NMR (CDCl₃) δ: 1.18 (3H, d, J=6.8 Hz), 3.27-3.35 (1H, m), 3.98 (1H, t, J=10.0 Hz), 4.24-4.31 (2H, m), 4.42 (1H, q, J=6.8 Hz), 4.71 (1H, dd, J=46.4, 9.4 Hz), 4.85 (1H, dd, J=46.4, 9.4 Hz), 7.15-7.26 (2H, m), 7.56-7.35 (5H, m), 8.27 (2H, d, J=7.4 Hz), 12.14 (1H, s).

### Step 8: Synthesis of Compound 3-10

To a solution of Compound 3-9 (1.04 g, 2.69 mmol) in MeOH (10 ml) and THF (10 ml) was added hydrazine hydrate (1.31 ml, 26.9 mmol) at room temperature. After stirring for 1 hour at 50° C., the reaction mixture was concentrated. The resulting residue was added to an amino silica gel column and eluted with Hexane/EtOAc 50% to 80%. Collected fractions were evaporated to afford Compound 3-10 (734 mg, 2.60 mmol, 97%) as a white solid.

¹H NMR (CDCl₃) δ: 1.11 (3H, d, J=6.8 Hz), 3.16-3.24 (1H, m), 3.89 (1H, t, J=9.9 Hz), 3.95 (1H, d, J=4.4 Hz), 4.17-4.30 (3H, m), 4.51 (1H, dd, J=9.2, 47.1 Hz), 4.66 (1H, ddd, J=47.1, 9.0, 1.5 Hz), 7.07 (1H, dd, J=12.3, 7.7 Hz), 7.18 (1H, t, J=7.2 Hz), 7.27-7.35 (1H, m), 7.51-7.44 (1H, m).

### Step 9: Synthesis of Compound 3-11

To a solution of Compound 3-10 (734 mg, 2.60 mmol) in TFA (5.6 ml) was added sulfuric acid (1.39 ml, 26.0 mmol) at −8° C. After stirring for 5 minutes at the same temperature, to the reaction mixture was added HNO₃ (0.174 ml, 3.90 mmol). After stirring for 10 minutes at the same temperature, the reaction mixture was treated with aqueous K₂CO₃ solution, and the aqueous layer was extracted with EtOAc. The organic layer was washed with water and was concentrated in vacuo. The crude product was added to an amino silica gel column and eluted with hexane/EtOAc 60%. Collected fractions were evaporated to afford Compound 3-11 (820 mg, 2.51 mmol, 96%) as a white amorphous.

¹H NMR (CDCl₃) δ: 1.14 (3H, d, J=6.7 Hz), 3.13-3.20 (1H, m), 3.88 (1H, t, J=9.8 Hz), 3.97 (1H, d, J=4.8 Hz), 4.09-4.23 (2H, m), 4.34 (2H, s), 4.48 (1H, dd, J=20.6, 8.2 Hz), 4.60 (1H, dd, J=20.6, 8.2 Hz), 7.23-7.28 (1H, m), 8.26-8.20 (1H, m), 8.45 (1H, dd, J=6.8, 2.8 Hz).

### Step 10: Synthesis of Compound 3-12

A solution of Compound 3-11 (820 mg, 2.51 mmol) and 10% Pd—C (169 mg, 0.0750 mmol) in MeOH (16 ml) was stirred under H2 atmosphere at room temperature. After stirring for 2 hours at the same temperature, the mixture was filtered through Celite (Registered trademark) pad. The filtrate was concentrated under vacuum. The resulting residue was purified by supercritical fluid chromatography (SFC) (Chiralpak (Registered trademark) ID; 20% isopropylalcohol with 0.1% diethylamine) to give Compound 3-12 (300 mg, 1.01 mmol, 40%).

¹H NMR (CDCl₃) δ: 1.11 (3H, d, J=6.8 Hz), 3.14-3.21 (1H, m), 3.61 (2H, s), 3.86 (1H, t, J=9.9 Hz), 4.01 (1H, d, J=4.3 Hz), 4.08-4.20 (2H, m), 4.47 (1H, dd, J=46.9, 8.8 Hz), 4.63 (1H, ddd, J=46.9, 8.8, 1.6 Hz), 6.60-6.55 (1H, m), 6.75 (1H, dd, J=6.7, 2.9 Hz), 6.86 (1H, dd, J=11.9, 8.7 Hz).

### Step 11: Synthesis of Compound I-003

To a solution of Compound 3-12 (20.0 mg, 0.0670 mmol) in MeOH (1 ml) were added 5-(fluoromethoxy)pyrazine-2-carboxylic acid (11.6 mg, 0.0670 mmol) and 2 mol/L HCl (0.0336 ml, 0.0670 mmol) at room temperature. To the reaction mixture was added EDC-HCl (19.7 mg, 0.0740 mmol) at the same temperature. After stirring for 1 hour, the reaction mixture was treated with aqueous NaHCO₃. The aqueous layer was extracted with AcOEt, and the organic layer was dried over Na₂SO₄, filtered and concentrated to afford Compound I-003 (22.0 mg, 0.0490 mmol, 72%) as a white solid.

¹H NMR (CDCl₃) δ: 1.13 (3H, d, J=6.7 Hz), 3.18-3.25 (1H, m), 3.89 (1H, t, J=9.9 Hz), 4.03 (1H, d, J=4.5 Hz), 4.13 (1H, q, J=6.7 Hz), 4.17-4.24 (1H, m), 4.33 (2H, br s), 4.46-4.71 (2H, m), 6.15 (2H, ddd, J=51.1, 7.1, 2.0 Hz), 7.13 (1H, dd, J=11.7, 8.9 Hz), 7.54 (1H, dd, J=6.9, 2.8 Hz), 7.97-8.02 (1H, m), 8.29 (1H, d, J=1.3 Hz), 9.08 (1H, d, J=1.3 Hz), 9.51 (1H, s).

### Example 4

### Synthesis of Compound I-004

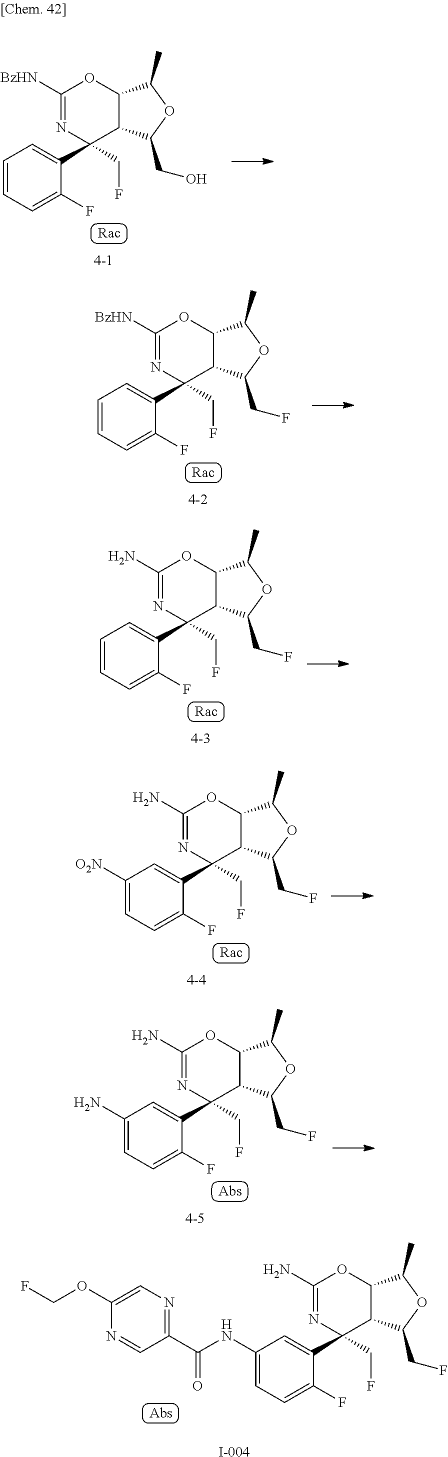

### Step 1: Synthesis of Compound 4-1

Compound 4-1 was synthesized from Compound 3-1 in a manner similar to the above protocols (Example 1, step 1 to step 12)

¹H NMR (CDCl₃) δ: 1.18 (3H, d, J=6.8 Hz), 3.49 (1H, dd, J=9.9, 4.3 Hz), 3.68-3.74 (1H, m), 4.02 (1H, d, J=10.4 Hz), 4.28 (1H, d, J=4.3 Hz), 4.39 (1H, d, J=9.9 Hz), 4.48 (1H, q, J=6.8 Hz), 4.75-4.86 (1H, m), 4.87-4.96 (1H, m), 7.14-7.26 (2H, m), 7.34-7.58 (4H, m), 7.62-7.72 (1H, m), 8.27 (2H, d, J=7.0 Hz).

### Step 2: Synthesis of Compound 4-2

To a solution of Compound 4-1 (165 mg, 0.40 mmol) in CH₂Cl₂ (10 ml) was added DAST (128 mg, 0.79 mmol) at 0° C. under nitrogen. The mixture was stirred at 0° C. for 2 hours. After stirring for 1 hour at room temperature, the reaction mixture was quenched by saturated aqueous NaHCO₃ solution, and the mixture was extracted with EtOAc. The organic layer was washed with water and brine, dried over Na₂SO₄ and concentrated in vacuo. The crude product was added to a silica gel column and eluted with hexane/EtOAc 50%. Collected fractions were evaporated to afford Compound 4-2 as white solid, which was used for the next step without purification.

MS (method B): m/z=417 [M+H]⁺

### Step 3: Synthesis of Compound 4-3

The Compound 4-3 was prepared in a manner similar to the above protocols (Example 1, step 15). (yield; crude)

MS (method B): m/z=315 [M+H]⁺

### Step 4 Synthesis of Compound 4-4

The Compound 4-4 was prepared in a manner similar to the above protocols (Example 1, step 16). (yield; crude)

MS (method B): m/z=360 [M+H]⁺

### Step 5 Synthesis of Compound 4-5

The Compound 4-5 was prepared in a manner similar to the above protocols (Example 1, step 17, SFC; Chiralpak (Registered trademark) ID, 10% methanol with 0.1% diethylamine). (yield; 20%, 4 steps).

MS (method B): m/z=330 [M+H]⁺

### Step 5: Synthesis of Compound I-004

The Compound I-004 was prepared in a manner similar to the above protocols (Example 1, step 18). (yield; 70%)

MS (method B): m/z=484 [M+H]⁺

¹H NMR (CDCl₃) δ: 1.13 (3H, d, J=6.8 Hz), 3.39 (1H, dd, J=9.7, 4.5 Hz), 4.10 (1H, d, J=4.5 Hz), 4.38-4.49 (3H, m), 4.55 (1H, d, J=9.3 Hz), 4.64-4.70 (1H, m), 4.72-4.89 (2H, m), 6.09 (1H, d, J=3.9 Hz), 6.22 (1H, d, J=3.9 Hz), 7.10-7.16 (1H, m), 7.56 (1H, dd, J=6.8, 2.7 Hz), 7.94-7.98 (1H, m), 8.30 (1H, s), 9.08 (1H, s), 9.52 (1H, s).

### Example 5

### Synthesis of Compound I-005

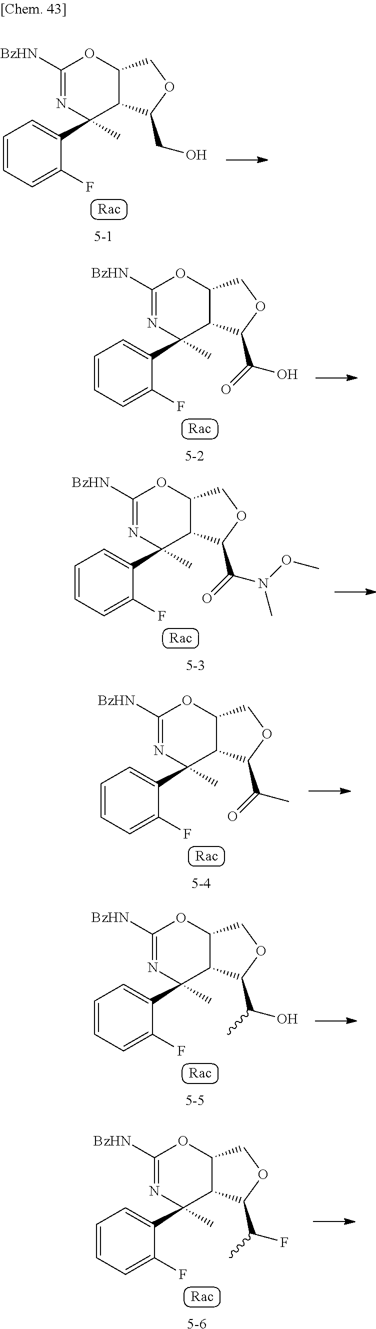

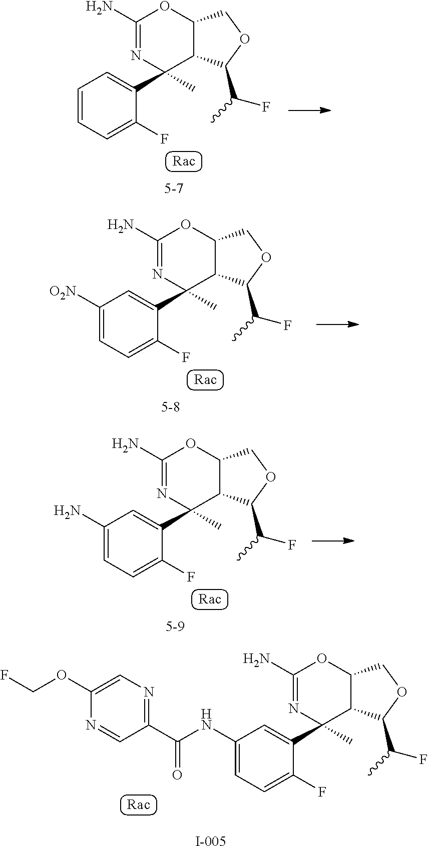

### Step 1: Synthesis of Compound 5-1

Compound 5-1 was synthesized from Compound 1-1 in a manner similar to the above protocols (Example 1, step 1 to step 12)

¹H NMR (CDCl₃) δ: 1.78 (3H, s), 2.01 (1H, t, J=6.5 Hz), 3.39 (1H, dd, J=8.8, 4.5 Hz), 3.74-3.81 (1H, m), 3.92 (1H, dd, J=10.5, 2.5 Hz), 4.00-4.07 (1H, m), 4.24 (1H, d, J=10.5 Hz), 4.28-4.34 (1H, m), 4.58 (1H, dd, J=4.5, 2.5 Hz), 7.10-7.19 (2H, m), 7.25-7.30 (1H, m), 7.32-7.38 (1H, m), 7.44 (2H, t, J=7.4 Hz), 7.52 (1H, t, J=7.4 Hz), 8.23-8.29 (2H, m), 11.70 (1H, s).

### Step 2: Synthesis of Compound 5-2

To a solution of Compound 5-1 (165 mg, 0.59 mmol) in EtOAc (6 ml) were added (1r,3r,5r,7r)-2-(11-oxidanyl)-2-azaadamantane (AZADO, 0.9 mg, 6 μmol), potassium bromide (14.1 mg, 0.12 mmol), and NaHCO₃(74.8 mg, 0.89 mmol) in H₂O (3 ml) at 0° C. After stirring for at 0° C. for 30 minutes, the reaction mixture was quenched by Na₂S₂O₃ 5H₂O (294 mg, 1.19 mmol, 2.0 eq) and NaCl (295 mg, 5.04 mmol, 8.5 eq) in H₂O (10 ml), and the mixture was extracted with EtOAc. The organic layer was washed with brine, dried over MgSO₄ and concentrated in vacuo to afford Compound 5-2 as a white solid, which was used for the next step without purification.

MS (method B): m/z=399 [M+H]⁺

### Step 3: Synthesis of Compound 5-3

To a solution of Compound 5-2 in CH₂Cl₂ (8.6 ml) were added N,O-dimethylhydroxylamine hydrochloride (105 mg, 1.075 mmol), N-ethyl-N-isopropylpropan-2-amine (188 μl, 1.075 mmol), and 3-(((ethylimino)methylene)amino)-N,N-dimethylpropan-1-amine hydrochloride (113 mg, 0.591 mmol) at room temperature. After stirring for at room temperature for 30 minutes, the reaction mixture was quenched by H₂O, and the mixture was extracted with EtOAc. The organic layer was washed with brine, dried over Na₂SO₄ and concentrated in vacuo to afford Compound 5-3 as an yellow oil, which was used for the next step without purification.

MS (method B): m/z=442 [M+H]⁺

### Step 4: Synthesis of Compound 5-4

To a solution of Compound 5-3 in THF (10.5 ml) was added methylmagnesium bromide (0.789 mL, 2.367 mmol) at 0° C. under nitrogen. After stirring for at 0° C. for 1 hour, the reaction mixture was quenched by saturated aqueous NH₄Cl solution, and the mixture was extracted with EtOAc. The organic layer was washed with brine, dried over Na₂SO₄ and concentrated in vacuo to afford Compound 5-4 as an yellow oil, which was used for the next step without purification.

MS (method B): m/z=397 [M+H]⁺

### Step 5: Synthesis of Compound 5-5

To a solution of Compound 5-34 in MeOH (8 ml) was added sodium borohydride (38.8 mg, 1.027 mmol) at 0° C. After stirring for at 0° C. for 1 hour, the reaction mixture was quenched by saturated aqueous NH₄Cl solution, and the mixture was extracted with CH₂C12. The organic layer was washed with brine, dried over Na₂SO₄ and concentrated in vacuo to afford Compound 5-5 as an yellow oil, which was used for the next step without purification.

MS (method B): m/z=399 [M+H]⁺

### Step 6: Synthesis of Compound 5-6

The Compound 6-6 was prepared in a manner similar to the above protocols (Example 3, step 2). (yield; 34%)

MS (method B): m/z=401 [M+H]⁺

¹H NMR (CDCl₃) δ: 1.53 (3H, d, J=6.3 Hz), 1.75 (3H, s), 3.46 (1H, t, J=6.3 Hz), 3.83 (1H, d, J=10.7 Hz), 4.22 (1H, d, J=22.1 Hz), 4.55-4.82 (2H, m), 5.44 (1H, dd, J=50.3, 13.7 Hz), 7.11-7.17 (2H, m), 7.23-7.28 (1H, m), 7.32-7.36 (1H, m), 7.42-7.45 (2H, m), 7.50-7.52 (1H, m), 8.26-8.27 (2H, m), 11.54 (1H, s).

Step 7: Synthesis of compound 5-7

The Compound 5-7 was prepared in a manner similar to the above protocols (Example 1, step 15). (yield; crude)

MS (method B): m/z=297 [M+H]⁺

Step 8: Synthesis of compound 5-8

The compound 5-8 was prepared in a manner similar to the above protocols (Example 1, step 16). (yield; crude)

MS (method B): m/z=342 [M+H]⁺

### Step 9: Synthesis of Compound 5-9

The Compound 5-9 was prepared in a manner similar to the above protocols (Example 1, step 17). (yield; 80%, 3 steps)

MS (method B): m/z=312 [M+H]⁺

### Step 9: Synthesis of Compound I-005

The Compound I-005 was prepared in a manner similar to the above protocols (Example 1, step 18, SFC; Chiralpak (Registered trademark) IE, 35% MeOH with 0.1% diethylamine). (yield; 25%). This compound was diastereomer mixture (ratio: 1:0.3).

MS (method B): m/z=466 [M+H]⁺

¹H NMR (CDCl₃) δ: 1.44 (0.9H, d, J=6.4 Hz), 1.50 (3H, d, J=6.4 Hz), 1.58 (3.9H, s), 1.59-1.96 (0.3H, br m), 2.88 (0.3H, dd, J=8.6, 4.3 Hz), 3.14 (1H, dd, J=8.6, 4.3 Hz), 3.76 (1.3H, d, J=10.3 Hz), 3.90 (0.3H, d, J=10.3 Hz), 3.97 (1H, d, J=10.3 Hz), 4.05-4.07 (0.3H, m), 4.21-4.29 (1H, m), 4.29-4.32 (0.3H, br m), 4.37-4.39 (1H, br m), 4.74-4.86 (1H, m), 6.08 (1.3H, d, J=2.7 Hz), 6.21 (1.3H, d, J=2.7 Hz), 7.08-7.11 (1.3H, m), 7.37-7.40 (1.3H, m), 7.93-7.97 (1.3H, m), 8.29 (1.3H, d, J=1.3 Hz), 9.08 (1.3H, s), 9.49 (1.3H, s).

### Example 6

### Synthesis of Compound I-006

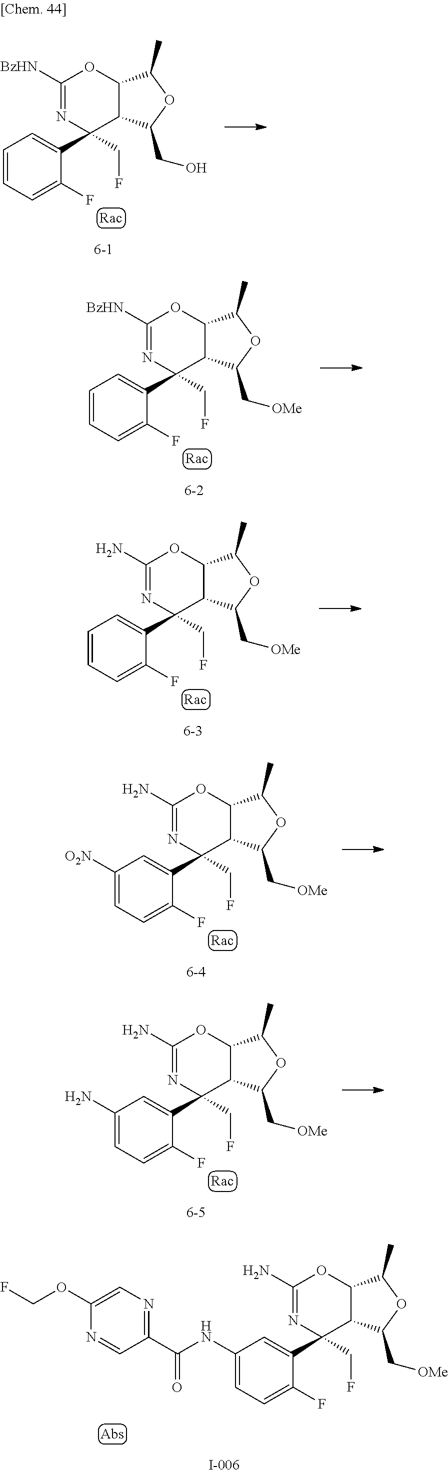

### Step 1: Synthesis of Compound 6-2

To a solution of compound 6-1 (198 mg, 0.48 mmol) in DMF (6 ml) was added NaH (48 mg, 1.19 mmol) at room temperature. After stirring for 30 minutes at room temperature, MeI (68 mg, 0.48 mmol) was added to the mixture. After stirring overnight at room temperature, the reaction mixture was quenched by water, and the mixture was extracted with EtOAc. The organic layer was washed with brine, dried over Na₂SO₄ and concentrated in vacuo. The crude product was added to a NH silica gel column and eluted with hexane/EtOAc 40%. Collected fractions were evaporated to afford Compound 6-2 as a colorless oil, which was used for the next step without purification.

MS (method B): m/z=431 [M+H]⁺

### Step 2: Synthesis of Compound 6-3

The Compound 6-3 was prepared in a manner similar to the above protocols (Example 1, step 15) (yield; crude)

MS (method B): m/z=327 [M+H]⁺

### Step 3: Synthesis of Compound 6-4

The Compound 6-4 was prepared in a manner similar to the above protocols (Example 1, step 16)(yield; crude)

MS (method B): m/z=372 [M+H]⁺

### Step 4: Synthesis of Compound 6-5

The Compound 6-5 was prepared in a manner similar to the above protocols (Example 1, step 17, SFC; Chiralpak (Registered trademark) IC, 10% MeOH with 0.1% diethylamine)(yield; 36%, 4 steps)

MS (method B): m/z=330 [M+H]⁺

### Step 5: Synthesis of Compound I-006

The Compound 6-6 was prepared in a manner similar to the above protocols (Example 1, step 18)(yield; 80%)

MS (method B): m/z=496 [M+H]⁺

¹H NMR (CDCl₃) δ: 1.13 (3H, d, J=6.8 Hz), 3.27 (1H, dd, J=10.7, 4.3 Hz), 3.47 (3H, s), 3.55 (1H, dd, J=10.7, 4.3 Hz), 3.73 (1H, d, J=10.7 Hz), 4.07 (1H, d, J=4.3 Hz), 4.44-4.64 (5H, m), 6.09 (1H, d, J=4.9 Hz), 6.21 (1H, d, J=4.9 Hz), 7.11-7.14 (1H, m), 7.51 (1H, dd, J=6.7, 2.8 Hz), 7.96-8.00 (1H, m), 8.29 (1H, s), 9.08 (1H, s), 9.51 (1H, s).

### Example 7

### Synthesis of Compound I-013

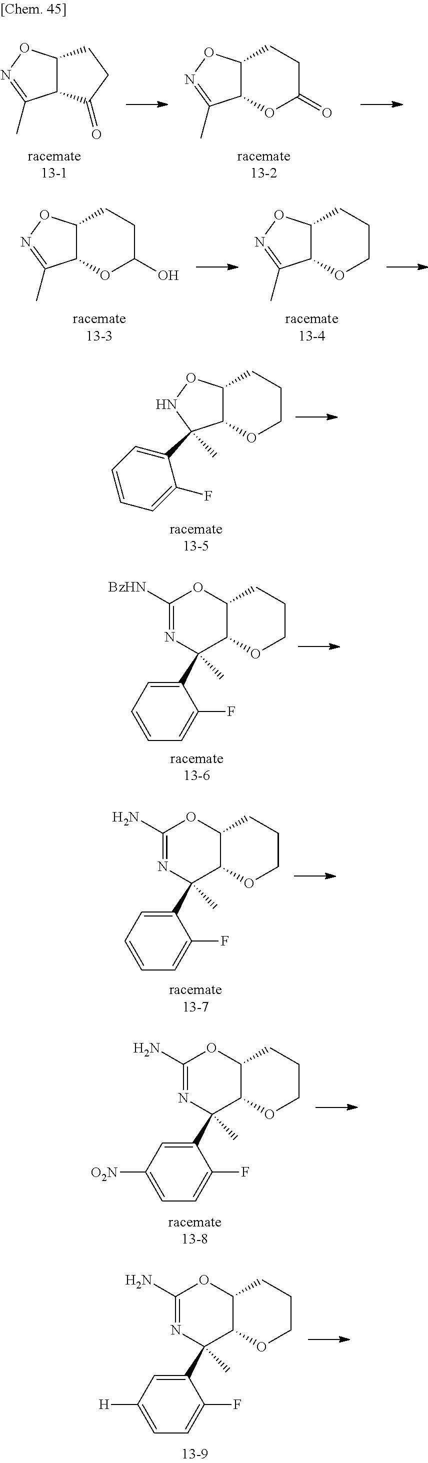

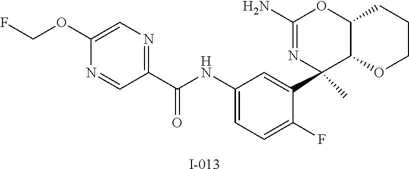

### Step 1: Synthesis of Compound 13-2

To a solution of Compound 13-1 (42.3 g, 304 mmol), which was prepared according to the known procedure (WO2014001228), in CH₂Cl₂ (423 ml) was added mCPBA (70 wt. %, 74.9 g, 304.0 mmol) at 0° C. After stirring overnight at room temperature, aqueous Na₂S₂O₃ solution was added to the reaction mixture. The reaction mixture was concentrated in vacuo. The residue was extracted with EtOAc. The organic layer was washed with brine, dried over MgSO₄ and concentrated in vacuo. The crude product was added to a silica gel column and eluted with hexane/EtOAc. Collected fractions were evaporated to afford Compound 13-2 (42.0 g, 271 mmol, 89%) as a colorless oil.

¹H NMR (CDCl₃) δ: 2.08-2.17 (4H, m), 2.34 (1H, ddd, J=14.7, 7.5, 3.8 Hz), 2.43-2.57 (2H, m), 4.80-4.76 (1H, m), 5.54 (1H, d, J=9.0 Hz).

### Step 2: Synthesis of Compound 13-3

To a solution of Compound 13-2 (13.4 g, 86 mmol) in toluene (134 ml) was added DIBAL (1.0 M in n-hexane, 95.0 mL, 95.0 mmol) at −78° C. The mixture was stirred for 1 hour at the same temperature. The reaction mixture was treated with aqueous Rochelle's salt solution. The insoluble materials were filtered off through a pad of Celite (Registered trademark). To the filtrate was added sodium chloride, and the aqueous layer was extracted with EtOAc. The organic layer was extracted with EtOAc and dried over MgSO₄ and concentrated in vacuo to afford the crude Compound 13-3 (4.05 g, 30%) as diastereomer mixtures.

### Step 3: Synthesis of Compound 13-4

To a solution of Compound 13-3 (3.0 g, 19.1 mmol) in CH₂C12/CH₃CN (4.5 mL/4.5 mL) were added triethylsilane (6.66 g, 57.3 mmol) and BF₃-OEt₂ (8.13 g, 57.3 mmol) at 0° C., and the reaction mixture was stirred for 30 minutes at the same temperature. To the reaction mixture was added aqueous NaHCO₃ solution, and the aqueous layer was extracted with CH₂C12. The organic layer was washed with saturated aqueous NaCl solution, dried over MgSO₄ and concentrated in vacuo. The crude product was added to a silica gel column and eluted with hexane/EtOAc. Collected fractions were evaporated to afford Compound 13-4 (720 mg, 5.16 mmol, 27%).

¹H NMR (CDCl₃) δ: 1.54-1.62 (1H, m), 1.72-1.95 (2H, m), 2.09 (3H, s), 2.21-2.15 (1H, m), 3.46 (1H, ddd, J=12.2, 8.1, 3.2 Hz), 3.68-3.73 (1H, m), 4.15-4.18 (1H, m), 4.54 (1H, d, J=6.0 Hz).

Step 4: Synthesis of compound 13-5

To a solution of 1-bromo-2-fluorobenzene (2.23 g, 12.8 mmol) in toluene/THF (28.8 mL/5.76 mL) was added n-BuLi (1.55 M in n-hexane, 8.23 mL, 12.8 mmol) at −78° C., and the reaction mixture was stirred for 30 minutes at the same temperature. To the reaction mixture were added BF₃-OEt₂ (724 mg, 5.10 mmol) and a solution of Compound 13-4 in THF/toluene (0.72 mL/3.60 mL) at −78° C., and the reaction mixture was stirred for 1 hour at the same temperature. To the reaction mixture was added aqueous NH₄Cl solution, and the aqueous layer was extracted with EtOAc. The organic layer was washed with saturated aqueous NaHCO₃ and saturated aqueous NaCl solution, dried over MgSO₄ and concentrated in vacuo. The crude product was added to a silica gel column and eluted with hexane/EtOAc. Collected fractions were evaporated to afford Compound 13-5 (720 mg, 5.16 mmol, 27%).

¹H NMR (CDCl₃) δ: 1.42-1.38 (1H, m), 1.58 (3H, s), 1.60-1.69 (1H, m), 1.75-1.87 (1H, m), 1.99-2.05 (1H, m), 3.41-3.48 (1H, m), 3.57-3.59 (1H, m), 3.99-4.03 (1H, m), 4.25-4.27 (1H, m), 6.20 (1H, br s), 6.99-7.04 (1H, m), 7.13-7.18 (1H, m), 7.23-7.28 (1H, m), 7.91 (1H, td, J=7.9, 1.8 Hz).

### Step 5: Synthesis of Compound 13-6

To a solution of Compound 13-5 (1.2 g, 5.10 mmol) in acetic acid (12.1 mL) was added zinc (3.33 g, 51.0 mmol), and the reaction mixture was stirred for 2 hours at 60° C. The insoluble materials were filtered off through a pad of Celite (Registered trademark). The filtrate was concentrated in vacuo. To the residue was added aqueous NaHCO₃ solution, and the mixture was extracted with EtOAc. The organic layer was washed with brine, dried over MgSO₄ and concentrated in vacuo. To a solution of the residue in CH₂Cl₂ (10 mL) was added benzoyl isothiocyanate (641 mg, 3.93 mmol) at room temperature, and the reaction mixture was stirred for 1 hour. To the reaction mixture was added EDC (753 mg, 3.93 mmol) at room temperature and the reaction mixture was stirred for 1 hour. The reaction mixture was concentrated in vacuo. The crude product was added to a silica gel column and eluted with hexane/EtOAc. Collected fractions were evaporated to afford Compound 13-6 (1.1 g, 3.0 mmol, 59%). LC/MS: Method A, M+1=369, tR=1.80 min.

### Step 6: Synthesis of Compound 13-7

To a solution of Compound 13-6 (1.1 g, 3.0 mmol) in THF/MeOH (11 mL/11 mL) was added hydrazine hydrate (1.50 g, 30.0 mmol), and the reaction mixture was stirred at room temperature overnight. The reaction mixture was concentrated in vacuo, and the residue was added to silica gel column and eluted with hexane/EtOAc. Collected fractions were concentrated in vacuo to afford Compound 13-7 (1.3 g), which was used for the next reaction without further purification. LC/MS: Method A, M+1=265, tR=0.88 min.

### Step 7: Synthesis of Compound 13-8

To a solution of crude Compound 13-7 (1.3 g) in trifluoroacetic acid (9.7 mL) were added sulfuric acid (2.4 mL, 45 mmol) and nitric acid (0.40 mL, 9.00 mmol) at 0° C., and the reaction mixture was stirred for 30 minutes at the same temperature. The reaction mixture was poured into K₂CO₃ (1.6 g, 120 mmol) in ice water. The aqueous layer was extracted with EtOAc. The organic layer was washed with water, brine and dried over MgSO₄. The solvent was evaporated, added to a silica gel column and eluted with hexane/EtOAc. Collected fractions were evaporated to afford Compound 13-8 (927 mg, 3.0 mmol, quant).

¹H NMR (CDCl₃) δ: 1.39-1.42 (1H, m), 1.53-1.62 (1H, m), 1.59 (3H, d, J=1.9 Hz), 1.88-2.02 (2H, m), 3.53-3.60 (1H, m), 3.66-3.67 (1H, m), 3.86 (1H, s), 4.10-4.14 (2H, m), 4.23 (1H, br s), 7.17 (1H, dd, J=10.7, 8.9 Hz), 8.16 (1H, ddd, J=8.9, 4.0, 2.9 Hz), 8.41 (1H, dd, J=6.8, 2.9 Hz).

### Step 8: Synthesis of Compound 13-9

To a solution of crude Compound 13-8 (996 mg, 3.2 mmol) in MeOH (15 mL) was added Pd—C (5 wt. %, 343 mg, 0.16 mmol), and the reaction mixture was stirred for 1 hour at room temperature under hydrogen atmosphere. The insoluble materials were filtered off through a pad of Celite (Registered trademark). The filtrate was concentrated in vacuo. The crude was purified by supercritical fluid chromatography (SFC) (Chiralpak (Registered trademark) IC; 50% ethanol with 0.1% diethylamine) to afford Compound 13-9 (235 mg, 26%) as a single enantiomer. LC/MS: Method A, M+1=280, tR=0.77 min.

### Step 9: Synthesis of Compound I-013

To a solution of Compound 13-9 (50.0 mg, 0.179 mmol) in MeOH (1.0 mL) was added 2M aqueous hydrogen chloride (0.09 mL, 0.179 mmol). To the solution were added 5-(fluoromethoxy)pyrazine-2-carboxylic acid (30.8 mg, 0.179 mmol) and EDC (37.7 mg, 0.179 mmol) at room temperature and the reaction mixture was stirred for 1 hour. The reaction mixture was quenched with aqueous NaHCO₃ solution. The aqueous layer was extracted with EtOAc. The organic layer was washed with water and brine, dried over MgSO₄ and concentrated in vacuo. The crude product was triturated with MeOH/water to give Compound I-013 (58.0 mg, 75%).

¹H NMR (CDCl₃) δ: 1.25-1.42 (2H, m), 1.61 (3H, s), 1.87-2.01 (2H, m), 3.55-3.62 (1H, m), 3.81 (1H, s), 3.94 (1H, s), 4.09-4.13 (1H, m), 6.15 (3H, ddd, J=51.1, 9.4, 2.0 Hz), 7.07 (1H, dd, J=11.4, 8.8 Hz), 7.42 (1H, dd, J=6.8, 2.7 Hz), 8.01-8.05 (1H, m), 8.29 (1H, s), 9.08 (1H, s), 9.52 (1H, br s).

### Example 8

### Synthesis of Compound I-014

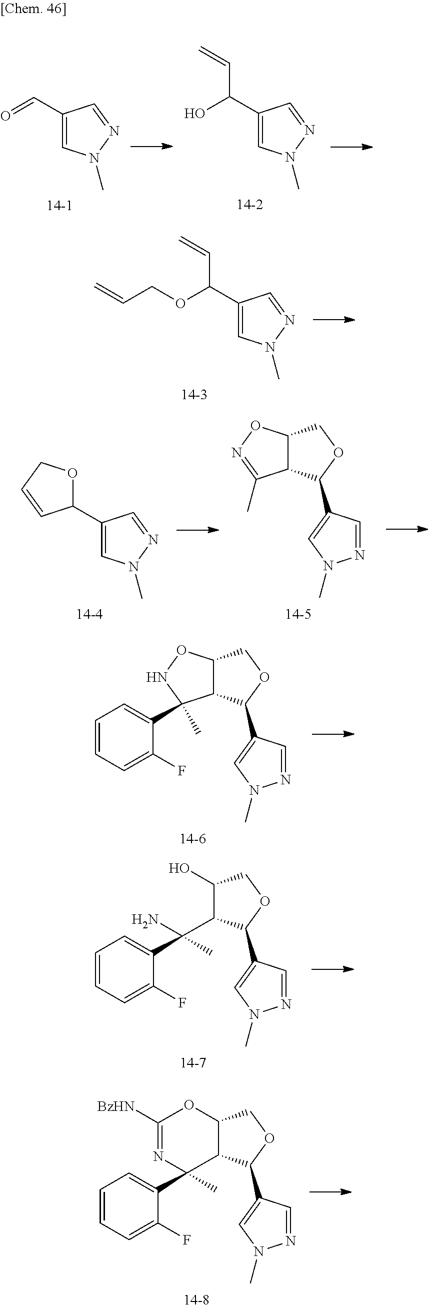

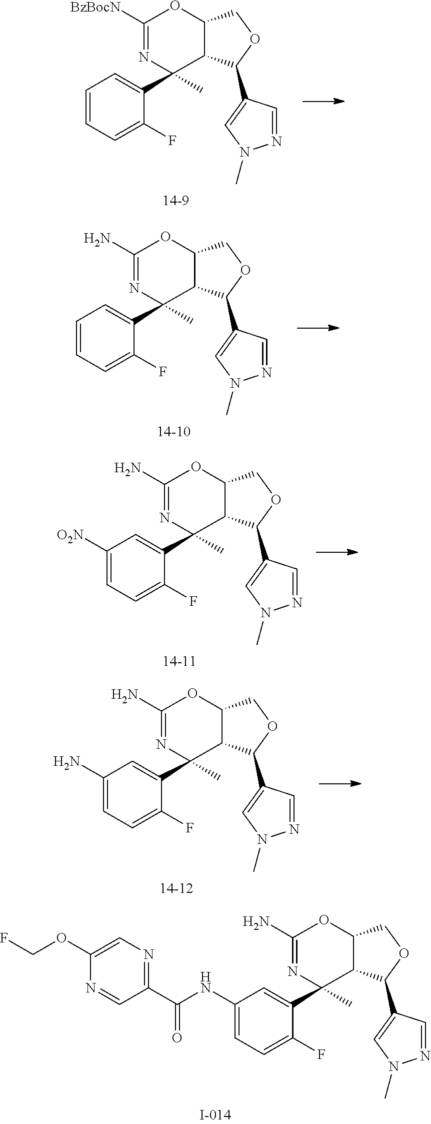

### Step 1

To a solution of Compound 14-1 (10.9 g, 99 mmol) in THF (219 mL) was added 1.45 mol/L of vinylmagnesium chloride in THF (82 mL, 119 mmol) at −78° C. After being stirred for 3 hours at −78° C., the reaction mixture was quenched with aqueous ammonium chloride, basified with potassium carbonate and extracted with ethyl acetate. The combined organic layers were washed with water. The solvent was evaporated and the residue was added to a amino silica gel column and eluted with hexane/EtOAc 20% to 100%. Collected fractions were evaporated to afford Compound 14-2 (9.97 g, 72.2 mmol, 73%) as a colourless oil.

¹H NMR (400 MHz, CDCl₃) δ: 1.76 (1H, d, J=6.4 Hz), 3.88 (3H, s), 5.18-5.23 (2H, m), 5.36 (1H, d, J=17.3 Hz), 6.09 (1H, ddd, J=17.1, 10.4, 6.0 Hz), 7.33 (1H, s), 7.45 (1H, s)

### Step 2

To a solution of Compound 14-2 (9.97 g, 72.2 mmol) in DMF (150 mL) was added 60 wt. % sodium hydride (4.33 g, 108 mmol). After being stirred for 10 minutes at room temperature, to the reaction mixture was added allyl bromide (12.5 mL, 144 mmol). After being stirred for 1 hour at room temperature, the reaction mixture was quenched with cold water and extracted with ethyl acetate. The combined organic layers were washed with water and brine, dried over sodium sulfate, and filtered. The solvent was evaporated. The crude product was added to a silica gel column and eluted with hexane/EtOAc 30% to 80%. Collected fractions were evaporated to afford Compound 14-3 (12.0 g, 67.3 mmol, 93%) as a white foam.

¹H NMR (400 MHz, CDCl₃) δ: 3.88 (3H, s), 3.94-4.05 (2H, m), 4.81 (1H, d, J=7.0 Hz), 5.15-5.33 (4H, m), 5.87-6.02 (2H, m), 7.31 (1H, s), 7.42 (1H, s).

### Step 3

To a solution of Compound 14-3 (12.0 g, 67.3 mmol) in dichloromethane (60 mL) was added [1,3-bis(2,4,6-trimethylphenyl)imidazolidin-2-ylidene](chloro)(phenylmethylidene)ruthenium; tricyclohexylphosphane (1.14 g, 1.35 mmol). After being stirred for 3 hours at 40° C., the solvent was evaporated. The crude product was added to a silica gel column and eluted with hexane/EtOAc 20% to 50%. Collected fractions were evaporated to afford Compound 14-4 (4.05 g, 27.0 mmol, 40%) as a white amorphous.

¹H NMR (400 MHz, CDCl₃) δ: 3.87 (3H, s), 4.64-4.70 (1H, m), 4.73-4.80 (1H, m), 5.78-5.82 (1H, m), 5.86-5.90 (1H, m), 6.02-6.05 (1H, m), 7.42 (1H, s), 7.45 (1H, s).

### Step 4

To a solution of Compound 14-4 (4.05 g, 27.0 mmol) in toluene (81 mL) were added nitroethane (5.81 mL, 81 mmol), isocyanatobenzene (11.7 mL, 108 mmol) and diisopropylethylamine (1.18 mL, 6.74 mmol). After being stirred for 10 hours at 130° C., the reaction mixture was cooled to room temperature and filtered. The filtrate was evaporate and added to a silica gel column and eluted with chloroform/methanol 0% to 20%. Collected fractions were evaporated to afford a crude product. The crude product was added to a silica gel column and eluted with hexane/EtOAc 30% to 70%. Collected fractions were evaporated to afford Compound 14-5 (2.75 g, 13.3 mmol, 49% as a mixture of isomers) as an orange solid.

¹H NMR (400 MHz, CDCl₃) δ: 1.98-2.08 (2H, m), 3.77-4.12 (6H, m), 5.10-5.30 (2H, m), 7.29-7.48 (2H, m).

### Step 5

To a solution of 1-bromo-2-fluorobenzene (11.6 g, 66.5 mmol) in toluene (110 mL) and THF (27.5 mL) was added 1.6 mol/L of n-butyl lithium in n-hexane (41.5 mL, 66.5 mmol) at −78° C. followed by boron trifluoride diethyl etherate (5.05 mL, 39.9 mmol) and a solution of Compound 14-5 (2.75 g, 13.3 mmol) in toluene (110 mL). After being stirred for 1 hour at −78° C., the reaction mixture was quenched with aqueous ammonium chloride solution and extracted with ethyl acetate. The combined organic layers were washed with brine, dried over sodium sulfate, and filtered. The solvent was evaporated. The crude product was added to a silica gel column and eluted with hexane/EtOAc 40% to 100%. Collected fractions were evaporated to afford Compound 14-6 (2.31 g, 7.62 mmol, 57% as a mixture of isomers) as a yellow amorphous. LC/MS: Method A, M+1=304, tR=1.37, 1.47 min.

### Step 6

To a solution of Compound 14-6 (2.31 g, 7.62 mmol) in acetic acid (23.1 mL) was added zinc (4.98 g, 76 mmol). After being stirred for 1 hour at 90° C., to the reaction mixture was added additional zinc (4.98 g, 76 mmol). After being stirred for 1 hour at 90° C., the reaction was quenched with 2 mol/L of aqueous sodium hydroxide solution (200 mL), and the mixture was diluted with ethyl acetate. The mixture was filtered through Celite (Registered trademark) pad. The filtrate was extracted with ethyl acetate. The organic layer was washed with brine, dried over sodium sulfate, and filtered to afford Compound 14-7 (2.02 g, 6.61 mmol, 87%) as a white amorphous. The product was used for the next reaction without further purification. LC/MS: Method A, M+1=306, tR=0.36 min.

### Step 7

To a solution of Compound 14-7 (2.02 g, 6.61 mmol) in dichloromethane (10.1 mL) was added benzoyl isothiocyanate (0.978 mL, 7.27 mL). After being stirred for 30 minutes at room temperature, to the reaction mixture was added EDC hydrochloride (1.52 g, 7.93 mmol). After being stirred 1 hour for 40° C., to the reaction mixture was added additional EDC hydrochloride (0.380 g, 1.98 mmol). After being stirred additional 1 hour for 40° C., the reaction mixture was evaporated. The residue was added to a silica gel column and eluted with hexane/EtOAc 10% to 100%. Collected fractions were evaporated to afford Compound 14-8 (1.08 g, 2.48 mmol, 38%) as a yellow amorphous.

¹H NMR (400 MHz, CDCl₃) δ: 3.47-3.53 (1H, m), 3.94 (3H, s), 4.05-4.15 (1H, m), 4.22 (1H, d, J=11.0 Hz), 4.64-4.68 (1H, m), 5.16 (1H, d, J=9.0 Hz), 7.09-7.56 (10H, m), 7.64 (1H, s), 8.28 (2H, d, J=7.3 Hz), 11.66-11.75 (1H, br).

### Step 8

To a solution of Compound 14-8 (1.07 g, 2.46 mmol) in THF (10.7 mL) were added di-tert-butyl dicarbonate (0.686 mL, 2.95 mmol) and DMAP (60.1 mg, 0.492 mmol). After being stirred for 3 hours at room temperature, the reaction mixture was evaporated. The residue was added to a silica gel column and eluted with hexane/EtOAc 50% to 100%. Collected fractions were evaporated to afford Compound 14-9 (839 mg, 1.57 mmol, 64%) as a white foam.

¹H NMR (400 MHz, CDCl₃) δ: 1.29 (3H, s), 1.46 (9H, s), 3.24 (1H, dd, J=10.3, 3.8 Hz), 3.92 (3H, s), 4.00-4.05 (2H, m), 4.51-4.53 (1H, m), 4.95 (1H, d, J=10.2 Hz), 7.05 (1H, ddd, J=12.7, 7.9, 1.1 Hz), 7.16 (1H, td, J=7.5, 1.2 Hz), 7.23-7.30 (1H, m), 7.43-7.52 (3H, m), 7.55-7.64 (3H, m), 7.81 (2H, d, J=6.9 Hz). (2.21 m, 535)

### Step 9

To a suspension of Compound 14-9 (839 mg, 1.57 mmol) in methanol (8.30 mL) was added potassium carbonate (325 mg, 2.35 mmol). After being stirred for 3 hours at room temperature, the reaction mixture was diluted with water and extracted with ethyl acetate. The combined organic layers were washed with brine, dried over sodium sulfate, and filtered. The solvent was evaporated. To a solution of the residue in dichloromethane (4.2 mL) was added TFA (4.2 mL, 54.5 mmol). After being stirred for 1 hour at room temperature, the reaction mixture was quenched with aqueous potassium carbonate solution and extracted with ethyl acetate. The combined organic layers were washed with brine, dried over sodium sulfate, and filtered. The residue was added to an amino silica gel column and eluted with chloroform/methanol 0% to 10%. Collected fractions were evaporated to afford Compound 14-10 (565 mg, 1.51 mmol, 96%) as a white foam.

¹H NMR (400 MHz, CDCl₃) δ: 1.39 (3H, s), 3.32 (1H, dd, J=9.3, 4.3 Hz), 3.89-3.97 (5H, m), 4.32-4.35 (1H, m), 5.07 (1H, d, J=9.4 Hz), 7.05 (1H, dd, J=12.5, 8.0 Hz), 7.13 (1H, t, J=7.5 Hz), 7.22-7.29 (1H, m), 7.38 (1H, td, J=8.0, 1.3 Hz), 7.46 (1H, s), 7.62 (1H, s).

### Step 10

To a solution of Compound 14-10 (565 mg, 1.51 mmol) in TFA (2.44 mL, 31.7 mmol) was added concentrated sulfuric acid followed by 70 wt. % nitric acid (0.116 mL, 1.81 mmol), and the mixture was stirred for 1 hour at −10° C. After being stirred for 1 hour at −10° C., the reaction mixture was quenched with aqueous potassium carbonate solution and extracted with ethyl acetate. The combined organic layers were washed with brine, dried over sodium sulfate, and filtered. The crude product was added to an amino silica gel column and eluted with chloroform/methanol 0% to 10%. Collected fractions were evaporated to afford Compound 14-11 (566 mg, 1.51 mmol, 100%) as a yellow solid.

¹H NMR (400 MHz, CDCl₃) δ: 1.39 (3H, s), 3.27 (1H, dd, J=9.3, 4.4 Hz), 3.94 (3H, s), 3.94-4.01 (2H, m), 4.32-4.36 (1H, m), 5.09 (1H, d, J=5.1 Hz), 7.21 (1H, dd, J=11.0, 9.0 Hz), 7.45 (1H, s), 7.61 (1H, s), 8.18 (1H, ddd, J=8.9, 4.0, 3.0 Hz), 8.35 (1H, dd, J=7.0, 2.9 Hz).

### Step 11

To a suspension of Compound 14-11 (566 mg, 1.51 mmol) in methanol (11.3 mL) was added concentrated hydrochloric acid (1.51 mL, 18.1 mmol) followed by zinc (690 mg, 10.6 mmol) at 0° C. After being stirred for 1.5 hours at 0° C., the reaction mixture was diluted with water and ethyl acetate, and basified with 2 mol/L of aqueous sodium hydroxide solution. The combined organic layers were washed with brine, dried over sodium sulfate, and filtered to afford Compound 14-12 (372 mg, 1.08 mmol, 71%) as a white amorphous. The product was used for the next reaction without further purification.

¹H NMR (400 MHz, CDCl₃) δ: 1.35 (3H, s), 3.30-3.35 (1H, m), 3.51-3.69 (2H, m), 3.89-3.98 (5H, m), 4.39-4.44 (1H, m), 5.03 (1H, d, J=9.3 Hz), 6.49-6.54 (1H, m), 6.66-6.71 (1H, m), 6.84 (1H, J=9.3 Hz), 7.45 (1H, s), 7.60 (1H, s). (0.38 min, 346)

### Step 12

To a suspension of Compound 14-12 (86 mg, 0.25 mmol) and 5-(fluoromethoxy)pyrazine-2-carboxylic acid (47.3 mg, 0.275 mmol) in methanol (1.73 mL) were added concentrated hydrochloric acid (0.125 mL, 0.25 mmol) and EDC hydrochloride (57.5 mg, 0.30 mmol). After being stirred for 3 hours at room temperature, the reaction mixture was diluted with aqueous potassium carbonate solution and extracted with ethyl acetate. The combined organic layers were washed with brine, dried over sodium sulfate, and filtered. The crude was purified by supercritical fluid chromatography (SFC) (Chiralpak (Registered trademark) IC; 30% ethanol with 0.1% diethylamine) to afford Compound I-014 (45.3 mg, 0.091 mmol, 36% yield) as a white solid.

¹H NMR (400 MHz, CDCl₃) δ: 1.62 (3H, s), 3.31-3.38 (1H, m), 3.91-3.97 (5H, m), 4.39-4.45 (1H, m), 5.07 (1H, d, J=9.7 Hz), 6.15 (2H, d, J=50.6 Hz), 7.09 (1H, t, J=10.2 Hz), 7.42-7.47 (2H, m), 7.62 (1H, s), 7.92-7.97 (1H, m), 8.29 (1H, s), 9.08 (1H, s), 9.49 (1H, s).

### Example 9

### Synthesis of Compound I-015

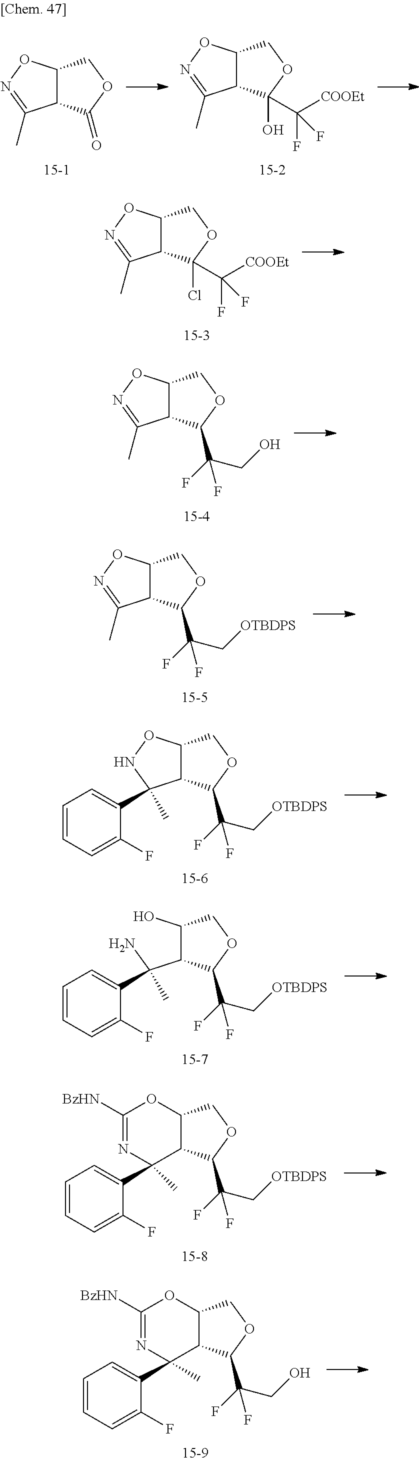

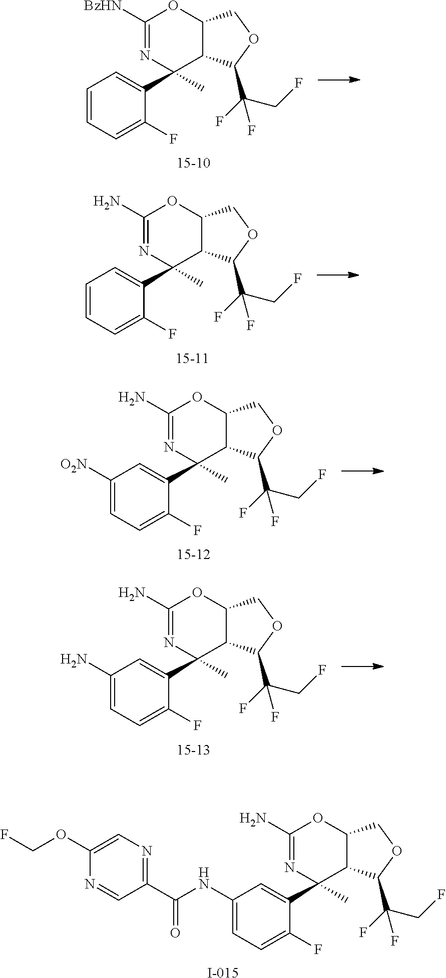

### Step 1

To a suspension of zinc (40.6 g, 621 mmol) in THF (313 mL) was added di(cyclopentadienyl)titanium dichloride (1.10 g, 4.43 mmol). After being stirred for 10 minutes at room temperature, Compound 15-1 (12.5 g, 89 mmol) and ethyl 2-bromo-2,2-difluoroacetate (22.6 ml, 177 mmol) were added to the reaction mixture. After being stirred for 8 hours at room temperature, the reaction mixture was filtered. The filtrate was diluted with ethyl acetate and aqueous ammonium chloride solution. The suspension was filtered through Celite (Registered trademark) pad. The filtrate was extracted with ethyl acetate. The combined organic layers were washed with water-brine (1:1) and brine, dried over sodium sulfate, and filtered. The solvent was evaporated. The crude product was added to a silica gel column and eluted with hexane/EtOAc 10% to 70%. Collected fractions were evaporated to afford Compound 15-2 (6.32 g, 23.8 mmol, 27%) as a yellow oil.

¹H NMR (400 MHz, CDCl₃) δ: 1.40 (3H, t, J=7.2 Hz), 2.08 (3H, s), 4.12 (1H, s), 4.18 (1H, d, J=10.4 Hz), 4.26-4.36 (2H, m), 4.42 (2H, q, J=7.2 Hz), 5.25-5.31 (1H, m).

### Step 2

To a solution of Compound 15-2 (6.32 g, 23.8 mmol) in dichloromethane (63.2 mL) was added thionyl chloride (3.48 mL, 47.6 mmol) at −10° C. followed by pyridine (5.78 mL, 71.5 mmol). After being stirred for 20 minutes at −10° C., the reaction mixture was quenched with iced water and extracted with chloroform. The combined organic layers were washed with brine, dried over sodium sulfate, and filtered. The solvent was evaporated. The crude product was added to a silica gel column and eluted with hexane/EtOAc 10% to 15%. Collected fractions were evaporated to afford Compound 15-3 (6.23 g, 22.0 mmol, 92%) as a yellow oil.

¹H NMR (400 MHz, CDCl₃) δ: 1.39 (3H, t, J=7.2 Hz), 2.12 (3H, s), 4.37-4.46 (3H, m), 4.51 (1H, dd, J=10.9, 4.3 Hz), 4.67 (1H, d, J=10.9 Hz), 5.42 (1H, dd, J=9.2, 4.3 Hz).

### Step 3

To a solution of Compound 15-3 (1.14 g, 4 mmol) and tributylstannane (2.91 g, 10.0 mmol) in methanol (5.6 mL) was added AIBN (32.8 mg, 0.200 mmol). After being stirred for 2 hours at 80° C., the reaction mixture was quenched with aqueous potassium fluoride solution. The suspension was filtered through Celite (Registered trademark) pad. Methanol (5.6 mL) was added to the organic layer of the filtrate. To the mixture was added sodium borohydride (378 mg, 10.0 mmol) at 0° C. After being stirred for 3 hours at room temperature, the reaction mixture was quenched with saturated aqueous ammonium chloride solution and extracted with ethyl acetate. The combined organic layers were washed with water and brine, dried over sodium sulfate, and filtered. The solvent was evaporated. The crude product was added to a silica gel column and eluted with hexane/EtOAc 30% to 70%. Collected fractions were evaporated to afford Compound 15-4 (500 mg, 2.41 mmol, 60%) as a yellow amorphous.

¹H NMR (400 MHz, CDCl₃) δ: 2.01-2.09 (4H, m), 3.86-4.03 (3H, m), 4.09-4.23 (2H, m), 4.41 (1H, d, J=26.1 Hz), 5.26-5.31 (1H, m).

### Step 4

To a solution of Compound 15-4 (495 mg, 2.39 mmol) in DMF (4.95 mL) were added t-butyldiphenylsilylchloride (0.737 mL, 2.87 mmol) and imidazole (244 mg, 3.59 mmol). After being stirred for 1 hour at room temperature, additional t-butyldiphenylsilylchloride (0.184 mL, 0.717 mmol) and imidazole (81 mg, 1.20 mmol) were added to the reaction mixture. After being stirred for additional 2 hours at room temperature, the reaction mixture was diluted with water and extracted with ethyl acetate. The combined organic layers were washed with water and brine, dried over sodium sulfate, and filtered. The solvent was evaporated. The crude product was added to a silica gel column and eluted with hexane/EtOAc 5% to 15%. Collected fractions were evaporated to afford Compound 15-5 (889 mg, 2.00 mmol, 83%) as a yellow foam.

¹H NMR (400 MHz, CDCl₃) δ: 1.06 (9H, s), 2.07 (3H, s), 3.80 (1H, ddd, J=12.9, 11.4, 5.0 Hz), 3.93-4.20 (4H, m), 4.55 (1H, d, J=26.2 Hz), 5.26 (1H, dd, J=9.3, 3.9 Hz), 7.38-7.48 (6H, m), 7.67 (4H, d, J=7.8 Hz).

### Step 5

The Compound 15-6 was prepared in a manner similar to the above protocols (Example 1, step 5). (yield: 100%)

LC/MS: Method A, M+1=542, tR=3.16 min.

### Step 6

The Compound 15-7 was prepared in a manner similar to the above protocols (Example 1, step 6). (yield: 34%) ¹H NMR (400 MHz, CDCl₃) δ: 1.01 (9H, s), 1.74 (3H, s), 3.36 (1H, dd, J=6.8, 4.6 Hz), 3.68 (1H, ddd, J=15.2, 11.3, 4.3 Hz), 3.79-3.86 (2H, m), 3.92-4.04 (2H, m), 4.36-4.50 (2H, m), 7.06-7.15 (2H, m), 7.23-7.33 (1H, m), 7.36-7.48 (7H, m), 7.61 (2H, d, J=6.9 Hz), 7.65 (2H, d, J=6.1 Hz).

### Step 7

The Compound 15-8 was prepared in a manner similar to the above protocols (Example 1, step 7). (yield: 86%)

LC/MS: Method A, M+1=673, tR=3.23 min.

### Step 8

To a solution of Compound 15-8 (396 mg, 0.589 mmol) in THF (3.96 mL) was added 1 mol/L of TBAF in THF (0.706 mL, 0.706 mmol). After being stirred at 1 hour at room temperature, the reaction mixture was diluted with water and extracted with ethyl acetate. The combined organic layers were washed with water and brine, dried over sodium sulfate, and filtered. The solvent was evaporated. The crude product was added to a silica gel column and eluted with hexane/EtOAc 30% to 70%. Collected fractions were evaporated to afford Compound 15-9 (244 mg, 0.562 mmol, 96%) as a yellow amorphous.

¹H NMR (400 MHz, CDCl₃) δ: 1.76 (3H, d, J=3.3 Hz), 2.15-2.22 (1H, br), 3.86-4.10 (4H, m), 4.23 (1H, dd, J=10.5, 2.1 Hz), 4.58 (1H, dd, J=24.7, 6.8 Hz), 4.66 (1H, dd, J=5.3, 1.9 Hz), 7.11-7.18 (2H, m), 7.23 (1H, td, J=8.1 Hz, 1.6 Hz), 7.31-7.39 (1H, m), 7.44 (2H, t, J=7.4 Hz), 7.50 (1H, t, J=7.3 Hz), 8.26 (2H, d, J=7.0 Hz), 11.44-11.57 (1H, br).

### Step 9

To a solution of Compound 15-9 (244 mg, 0.562 mmol) in dichloromethane (2.44 mL) was added DAST (0.445 mL, 3.37 mmol) at 0° C. After being stirred for 2 hours at room temperature and for 1 hour at 40° C., to the reaction mixture was added water (0.020 mL, 1.12 mmol). After being stirred for 3 hours at 40° C., the mixture was quenched with aqueous sodium bicarbonate solution and extracted with chloroform. The combined organic layers were washed with brine, dried over sodium sulfate, and filtered. The solvent was evaporated. To a solution of the residue in dichloromethane (2.44 mL) was added DAST (0.445 mL, 3.37 mmol) at 0° C. After being stirred for 15 hours at room temperature, the mixture was quenched with aqueous sodium bicarbonate and extracted with chloroform. The combined organic layers were washed with brine, dried over sodium sulfate, and filtered. The solvent was evaporated. The crude product was added to a silica gel column and eluted with hexane/EtOAc 10% to 50%. Collected fractions were evaporated to afford Compound 15-10 (158 mg, 0.361 mmol, 64%) as a yellow amorphous.

¹H NMR (400 MHz, CDCl₃) δ: 1.76 (3H, d, J=3.4 Hz), 3.86-3.93 (2H, m), 4.25 (1H, dd, J=10.4, 2.0 Hz), 4.48-4.89 (4H, m), 7.12-7.19 (2H, m), 7.23 (1H, td, J=8.2, 1.9 Hz), 7.33-7.39 (1H, m), 7.44 (2H, t, J=7.5 Hz), 7.52 (1H, t, J=7.4 Hz), 8.27 (2H, d, J=7.2 Hz), 11.49-11.57 (1H, br).

### Step 10

The Compound 15-11 was prepared in a manner similar to the above protocols the above protocols (Example 1, step 15). (yield: 93%) ¹H NMR (400 MHz, CDCl₃) δ: 1.59 (3H, s), 3.66-3.71 (1H, m), 3.79 (1H, d, J=10.0 Hz), 3.92 (1H, d, J=10.0 Hz), 4.35-4.85 (4H, m), 7.03-7.15 (2H, m), 7.22-7.36 (2H, m).

### Step 11

The Compound 15-12 was prepared in a manner similar to the above protocols the above protocols (Example 1, step 16). (yield: 99%) ¹H NMR (400 MHz, CDCl₃) δ: 1.57 (3H, s), 3.62-3.67 (1H, m), 3.81 (1H, d, J=10.5 Hz), 3.96 (1H, J=10.5 Hz), 4.24-4.93 (4H, m), 7.19-7.30 (1H, m), 8.16-8.22 (1H, m), 8.27-8.31 (1H, m).

### Step 12

The Compound 15-13 was prepared in a manner similar to the above protocols the above protocols (Example 1, step 17). (yield: 95%)

¹H NMR (400 MHz, CDCl₃) δ: 1.52 (3H, s), 3.52-3.60 (2H, br), 3.66 (1H, t, J=5.6 Hz), 3.78 (1H, d, J=10.0 Hz), 3.92 (1H, d, J=10.0 Hz), 4.34-4.84 (4H, m), 6.50-6.55 (1H, m), 6.59-6.64 (1H, m), 6.85 (1H, dd, J=11.3, 8.5 Hz).

### Step 13

The Compound I-015 was prepared in a manner similar to the above protocols the above protocols (Example 1, step 18, SFC; Chiralpak (Registered trademark) IC, 50% ethanol with 0.1% diethylamine) (yield: 37%).

¹H NMR (400 MHz, CDCl₃) δ: 1.58 (3H, s), 3.70 (1H, t, J=5.5 Hz), 3.81 (1H, d, J=9.8 Hz), 3.94 (1H, d, J=9.8 Hz), 4.39-4.86 (4H, m), 6.15 (2H, d, J=51.7 Hz), 7.10 (1H, t, J=9.9 Hz), 7.43-7.47 (1H, m), 7.85-7.91 (1H, m), 8.28 (1H, s), 9.07 (1H, s), 9.47 (1H, s).

The following compounds are prepared in a manner similar to the above. In the tables, tR means LC/MS retention time (minute).

TABLE 1

| No | Structure | ¹H NMR |
|---|---|---|
| I- 016 | 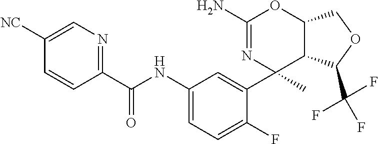 [SMILES: C[C@]1(c2cc(NC(=O)c3ccc(C#N)cn3)ccc2F)N=C(N)O[C@@H]2CO[C@@H](C(F)(F)F)[C@@H]21] | ¹H NMR (400 MHz, CDCl₃) δ: 1.60 (s, 3H), 3.54-3.57 (m, 1H), 3.90 (dd, J = 10.3, 1.5 Hz, 1H), 4.03 (d, J = 10.3 Hz, 1H), 4.43-4.49 (m, 2H), 7.13 (dd, J = 11.5, 8.9 Hz, 1H), 7.50 (dd, J = 7.2, 2.6 Hz, 1H), 7.90 (ddd, J = 8.9, 4.1, 2.6 Hz, 1H), 8.21 (dd, J = 8.0, 2.0 Hz, 1H), 8.43 (dd, J = 8.0, 0.9 Hz, 1H), 8.90 (dd, J = 2.0, 0.9 Hz, 1H), 9.84 (s, 1H). |
| I- 017 | 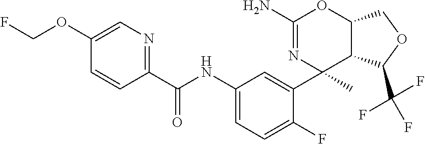 [SMILES: C[C@]1(c2cc(NC(=O)c3ccc(OCF)cn3)ccc2F)N=C(N)O[C@@H]2CO[C@@H](C(F)(F)F)[C@@H]21] | ¹H NMR (400 MHz, CDCl₃) δ: 1.59 (s, 3H), 3.54 (t, J = 5.7 Hz, 1H), 3.89 (d, J = 10.3 Hz, 1H), 4.02 (d, J = 10.3 Hz, 1H), 4.42-4.49 (m, 2H), 6.15 (dd, J = 51.1, 2.6 Hz, 2H), 7.10 (dd, J = 11.5, 8.8 Hz, 1H), 7.46 (dd, J = 7.0, 3.0 Hz, 1H), 7.87 (ddd, J = 8.8, 3.8, 3.0 Hz, 1H), 8.28 (d, J = 0.9 Hz, 1H), 9.07 (d, J = 0.9 Hz, 1H), 9.47 (s, 1H). |

TABLE 2

| | | |
|---|---|---|
| I- 018 | 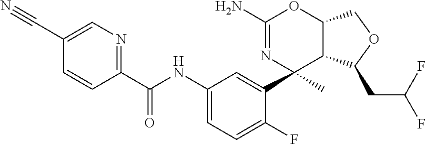 [SMILES: C[C@]1(c2cc(NC(=O)c3ccc(C#N)cn3)ccc2F)N=C(N)O[C@@H]2CO[C@@H](CC(F)F)[C@@H]21] | ¹H NMR (400 MHz, CDCl₃) δ: 1.57 (3H, s), 2.05-2.23 (1H, m), 2.31-2.45 (1H, m), 2.89 (1H, dd, J = 8.9, 4.4 Hz), 3.82 (1H, dd, J = 10.3, 2.1 Hz), 3.93 (1H, d, J = 10.4 Hz), 4.25- 4.32 (2H, m), 6.19 (1H, ddd, J = 56.8, 7.0, 2.4 Hz), 7.10 (1H, dd, J = 11.5, 8.8 Hz), 7.48 (1H, dd, J = 7.3, 2.8 Hz), 7.94 (1H, ddd, J = 8.7, 4.0, 2.9 Hz), 8.21 (1H, dd, J = 8.2, 2.0 Hz), 8.43 (1H, dd, J = 8.0, 0.6 Hz), 8.90 (1H, dd, J = 2.1, 0.7 Hz), 9.85 (1H, s). |
| I- 019 | 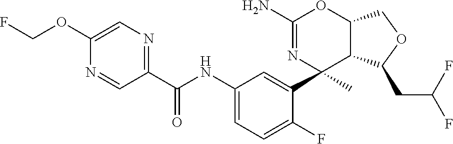 [SMILES: C[C@]1(c2cc(NC(=O)c3cnc(OCF)cn3)ccc2F)N=C(N)O[C@@H]2CO[C@@H](CC(F)F)[C@@H]21] | ¹H NMR (400 MHz, CDCl₃) δ: 1.57 (3H, s), 2.03-2.45 (2H, m), 2.88 (1H, dd, J = 8.0, 4.0 Hz), 3.82 (1H, d, J = 10.3 Hz), 3.92 (1H, d, J = 10.3 Hz), 4.29-4.33 (2H, m), 6.05 (1H, td, J = 56.9, 5.2 Hz), 6.15 (2H, dd, J = 51.4, 5.2 Hz), 7.09 (1H, dd, J = 11.3, 9.0 Hz), 7.40-7.45 (1H, m), 7.89-7.96 (1H, m), 8.29 (1H, s), 9.08 (1H, s), 9.49 (1H, s). |

TABLE 3

| | | |
|---|---|---|
| I- 020 | 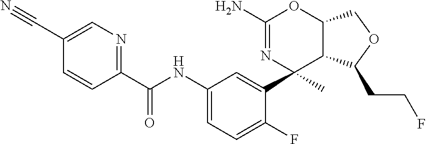 [SMILES: C[C@]1(c2cc(NC(=O)c3ccc(C#N)cn3)ccc2F)N=C(N)O[C@@H]2CO[C@@H](CCF)[C@@H]21] | ¹H NMR (400 MHz, CDCl₃) δ: 1.59 (3H, s), 1.86-2.05 (1H, m), 2.21-2.38 (1H, m), 2.93 (1H, dd, J = 8.8, 4.6 Hz), 3.80 (1H, dd, J = 10.3, 2.0 Hz), 3.92 (1H, d, J = 10.3 Hz), 4.24- 4.32 (2H, m), 4.62 (1H, dd, J = 7.5, 4.8 Hz), 4.74 (1H, dd, J = 7.5, 4.8 Hz), 7.10 (1H, dd, J = 10.7, 8.9 Hz), 7.46 (1H, dd, J = 7.0, 2.6 Hz), 7.95 (1H, ddd, J = 8.8, 4.0, 2.8 Hz), 8.21 (1H, dd, J = 8.2, 2.0 Hz), 8.43 (1H, d, J = 8.0 Hz), 8.91 (1H, dd, J = 1.6, 0.5 Hz), 9.86 (1H, s). |
| I- 021 | 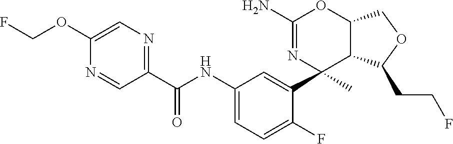 [SMILES: C[C@]1(c2cc(NC(=O)c3cnc(OCF)cn3)ccc2F)N=C(N)O[C@@H]2CO[C@@H](CCF)[C@@H]21] | ¹H NMR (400 MHz, CDCl₃) δ: 1.57 (3H, s), 1.85-2.06 (1H, m), 2.23-2.39 (1H, m), 2.91 (1H, dd, J = 8.3, 4.5 Hz), 3.79 (1H, d, J = 10.3 Hz), 3.91 (1H, d, J = 10.3 Hz), 4.22-4.32 (2H, m), 4.67 (2H, ddd, J = 46.7, 6.0, 4.7 Hz), 6.15 (2H, dd, J = 51.2, 4.0 Hz), 7.08 (1H, dd, J = 10.8, 8.8 Hz), 7.38- 7.43 (1H, m), 7.91-7.96 (1H, m), 8.29 (1H, s), 9.08 (1H, s), 9.49 (1H, s). |

TABLE 4

| | | |
|---|---|---|
| I- 022 | 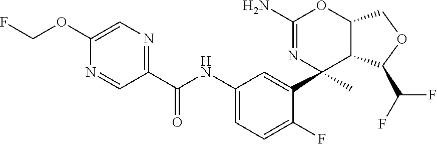 [SMILES: C[C@]1(c2cc(NC(=O)c3cnc(OCF)cn3)ccc2F)N=C(N)O[C@@H]2CO[C@@H](C(F)F)[C@@H]21] | ¹H NMR (400 MHz, CDCl₃) δ: 1.58 (3H, s), 3.46 (1H, dd, J = 7.0, 5.1 Hz), 3.83 (1H, dd, J = 10.3, 1.5 Hz), 3.99 (1H, d, J = 10.3 Hz), 4.25-4.36 (1H, m), 4.41 (1H, d, J = 4.3 Hz), 5.96 (1H, dd, J = 55.2, 1.6 Hz), 6.16 (2H, dd, J = 51.0, 2.9 Hz), 7.11 (1H, dd, J = 11.4, 8.7 Hz), 7.46 (1H, dd, J = 7.0, 2.8 Hz), 7.87-7.92 (1H, m), 8.29 (1H, s), 9.08 (1H, s), 9.49 (1H, s). |
| I- 023 | 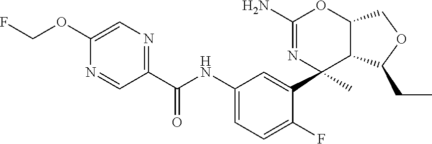 [SMILES: CC[C@@H]1OC[C@H]2OC(N)=N[C@](C)(c3cc(NC(=O)c4cnc(OCF)cn4)ccc3F)[C@@H]12] | ¹H NMR (400 MHz, CDCl₃) δ: 1.08 (3H, t, J = 7.3 Hz), 1.43-1.74 (1H, m), 1.56 (3H, s), 1.84-1.95 (1H, m), 2.85 (1H, dd, J = 8.5, 4.4 Hz), 3.76 (1H, dd, J = 10.3, 2.1 Hz), 3.89 (1H, d, J = 10.3 Hz), 4.03- 4.08 (1H, m), 4.27 (1H, dd, J = 4.4, 2.0 Hz), 6.15 (2H, ddd, J = 51.2, 5.3, 1.9 Hz), 7.08 (1H, dd, J = 11.6, 8.8 Hz), 7.38 (1H, dd, J = 7.1, 2.9 Hz), 7.96 (1H, ddd, J = 8.7, 3.9, 2.9 Hz), 8.29 (1H, d, J = 1.3 Hz), 9.08 (1H, d, J = 1.1 Hz), 9.49 (1H, s). |

TABLE 5

| | | |
|---|---|---|
| I- 024 | 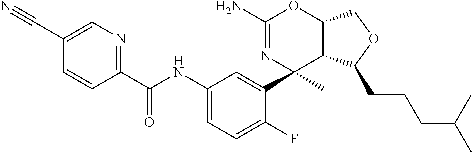 [SMILES: CC(C)CCC[C@@H]1OC[C@H]2OC(N)=N[C@](C)(c3cc(NC(=O)c4ccc(C#N)cn4)ccc3F)[C@@H]12] | ¹H NMR (400 MHz, CDCl₃) δ: 0.90 (6H, d, J = 6.7 Hz), 1.18-1.85 (10H, m), 2.84 (1H, dd, J = 8.4, 4.5 Hz), 3.76 (1H, dd, J = 10.2, 2.0 Hz), 3.89 (1H, d, J = 10.4 Hz), 4.07-4.14 (1H, m), 4.25-4.28 (1H, m), 7.10 (1H, dd, J = 11.6, 8.9 Hz), 7.43 (1H, dd, J = 7.0, 2.8 Hz), 7.96 (1H, ddd, J = 8.8, 3.9, 3.0 Hz), 8.20 (1H, dd, J = 8.2, 2.0 Hz), 8.43 (1H, d, J = 8.3 Hz), 8.91 (1H, s), 9.85 (1H, s). |
| I- 024 | 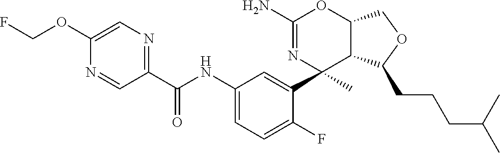 [SMILES: CC(C)CCC[C@@H]1OC[C@H]2OC(N)=N[C@](C)(c3cc(NC(=O)c4cnc(OCF)cn4)ccc3F)[C@@H]12] | ¹H NMR (400 MHz, CDCl₃) δ: 0.90 (6H, d, J = 6.5 Hz), 1.13-1.86 (7H, m), 1.58 (3H, s), 2.83 (1H, dd, J = 8.3, 4.3 Hz), 3.76 (1H, d, J = 10.3 Hz), 3.88 (1H, d, J = 10.3 Hz), 4.10 (1H, t, J = 8.3 Hz), 4.25-4.29 (1H, m), 6.15 (2H, d, J = 50.7 Hz), 7.08 (1H, dd, J = 11.3, 8.8 Hz), 7.30-7.39 (1H, m), 7.93-7.98 (1H, m), 8.29 (1H, s), 9.08 (1H, s), 9.49 (1H, s). |

TABLE 6

| | | |
|---|---|---|
| I- 027 | 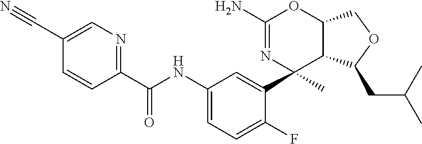 [SMILES: CC(C)C[C@@H]1OC[C@H]2OC(N)=N[C@](C)(c3cc(NC(=O)c4ccc(C#N)cn4)ccc3F)[C@@H]12] | ¹H NMR (400 MHz, CDCl₃) δ: 0.99 (6H, d, J = 6.7 Hz), 1.48-1.97 (3H, m), 1.54 (3H, s), 2.79 (1H, dd, J = 8.4, 4.6 Hz), 3.76 (1H, dd, J = 10.3, 2.0 Hz), 3.87 (1H, d, J = 10.3 Hz), 4.15-4.21 (1H, m), 4.25 (1H, dd, J = 4.4, 1.8 Hz), 7.09 (1H, dd, J = 11.5, 8.8 Hz), 7.43 (1H, dd, J = 7.0, 2.8 Hz), 7.95 (1H, ddd, J = 6.7, 3.9, 2.8 Hz), 8.20 (1H, dd, J = 8.0, 1.9 Hz), 8.43 (1H, dd, J = 8.2, 0.8 Hz), 8.90 (1H, dd, J = 1.9, 0.8 Hz), 9.84 (1H, s). |
| I- 028 | 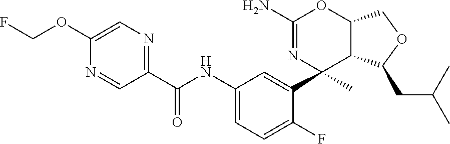 [SMILES: CC(C)C[C@@H]1OC[C@H]2OC(N)=N[C@](C)(c3cc(NC(=O)c4cnc(OCF)cn4)ccc3F)[C@@H]12] | ¹H NMR (400 MHz, CDCl₃) δ: 0.99 (6H, d, J = 6.8 Hz), 1.49-1.70 (2H, m), 1.56 (3H, s), 1.85-1.96 (1H, m), 2.78 (1H, dd, J = 8.4, 4.5 Hz), 3.75 (1H, dd, J = 10.2, 1.9 Hz), 3.87 (1H, d, J = 10.2 Hz), 4.18 (1H, t, J = 9.2 Hz), 4.25 (1H, dd, J = 4.3, 1.8 Hz), 6.15 (2H, ddd, J = 51.2, 4.8, 1.9 Hz), 7.08 (1H, dd, J = 11.7, 8.8 Hz), 7.38 (1H, dd, J = 7.3, 2.8 Hz), 7.91-7.97 (1H, m), 8.29 (1H, s), 9.08 (1H, s), 9.49 (1H, s). |

TABLE 7

| | | |
|---|---|---|
| I- 029 | 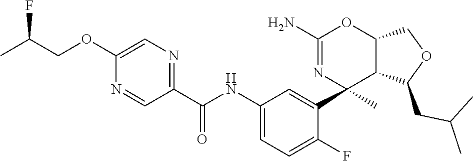 [SMILES: CC(C)C[C@@H]1OC[C@H]2OC(N)=N[C@](C)(c3cc(NC(=O)c4cnc(OC[C@@H](C)F)cn4)ccc3F)[C@@H]12] | ¹H NMR (400 MHz, CDCl₃) δ: 0.99 (6H, d, J = 6.7 Hz), 1.48 (3H, dd, J = 23.3, 6.4 Hz), 1.49- 1.66 (2H, m), 1.55 (3H, s), 1.83-1.96 (1H, m), 2.78 (1H, dd, J = 8.4, 4.5 Hz), 3.75 (1H, dd, J = 10.3, 2.0 Hz), 3.87 (1H, d, J = 10.3 Hz), 4.18 (1H, t, J = 9.4 Hz), 4.25 (1H, dd, J = 4.3, 1.6 Hz), 4.43-4.62 (2H, m), 4.96-5.16 (1H, m), 7.07 (1H, dd, J = 11.7, 8.7 Hz), 7.37 (1H, dd, J = 7.0, 2.8 Hz), 7.92- 7.97 (1H, m), 8.23 (1H, d, J = 1.1 Hz), 8.99 (1H, d, J = 1.1 Hz), 9.48 (1H, s). |
| I- 030 | 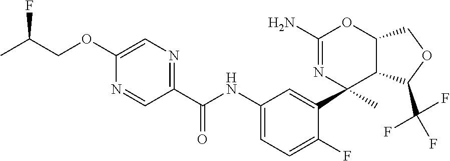 [SMILES: C[C@@H](F)COc1cnc(C(=O)Nc2ccc(F)c([C@@]3(C)N=C(N)O[C@@H]4CO[C@@H](C(F)(F)F)[C@@H]43)c2)cn1] | ¹H NMR (400 MHz, CDCl₃) δ: 1.48 (3H, dd, J = 23.5, 6.5 Hz), 1.58 (3H, s), 3.51-3.55 (1H, m), 3.89 (1H, dd, J = 10.3, 1.6 Hz), 4.02 (1H, d, J = 10.3 Hz), 4.41- 4.63 (4H, m), 4.95-5.17 (1H, m), 7.10 (1H, dd, J = 11.4, 8.7 Hz), 7.45 (1H, dd, J = 7.0, 2.8 Hz), 7.88 (1H, ddd, J = 9.0, 4.1, 3.0 Hz), 8.23 (1H, d, J = 1.1 Hz), 8.99 (1H, d, J = 1,1 Hz), 9.48 (1H, s). |

TABLE 8

| | | |
|---|---|---|
| I- 031 | 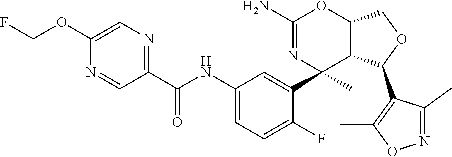 [SMILES: Cc1noc(C)c1[C@@H]1OC[C@H]2OC(N)=N[C@](C)(c3cc(NC(=O)c4cnc(OCF)cn4)ccc3F)[C@H]21] | ¹H NMR (400 MHz, CDCl₃) δ: 1.29 (3H, s), 2.38 (3H, s), 2.44 (3H, s), 3.35 (1H, dd, J = 10.5, 3.5 Hz), 4.01 (1H, d, J = 10.5 Hz), 4.07 (1H, d, J = 10.8, 2.0 Hz), 4.42-4.47 (1H, m), 4.90 (1H, d, J = 10.5 Hz), 6.16 (2H, dd, J = 51.2, 5.0 Hz), 7.07 (1H, t, J = 10.0 Hz), 7.46- 7.50 (1H, m), 7.87-7.93 (1H, m), 8.30 (1H, s), 9.09 (1H, s), 9.49 (1H, s). |
| I- 051 | 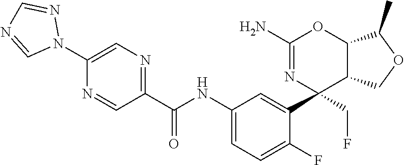 [SMILES: C[C@H]1OC[C@H]2[C@@H]1OC(N)=N[C@]2(CF)c1cc(NC(=O)c2cnc(-n3cncn3)cn2)ccc1F] | ¹H NMR (400 MHz, CDCl₃) δ: 1.14 (3H, d, J = 6.7 Hz), 3.19-3.27 (1H, m), 3.90 (1H, t, J = 9.9 Hz), 4.04 (1H, d, J = 4.4 Hz), 4.14 (1H, q, J = 6.7 Hz), 4.18-4.24 (1H, m), 4.31 (1H, br s), 4.46-4.72 (2H, m), 7.15 (1H, dd, J = 11.6, 8.8 Hz), 7.62-7.58 (1H, m), 8.05-7.98 (1H, m), 8.22 (1H, s), 9.25 (2H, s), 9.34 (1H, d, J = 1.1 Hz), 9.59 (1H, s). |

TABLE 9

| | | |
|---|---|---|
| I- 070 | 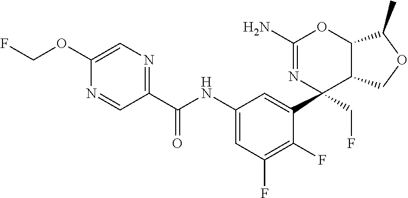 [SMILES: C[C@H]1OC[C@H]2[C@@H]1OC(N)=N[C@]2(CF)c1cc(NC(=O)c2cnc(OCF)cn2)cc(F)c1F] | ¹H NMR (400 MHz, CDCl₃) δ: 1.39 (3H, s), 3.31-3.37 (1H, m), 3.91-3.99 (2H, m), 3.93 (3H, s), 4.32-4.45 (5H, m), 5.07 (1H, d, J = 9.3 Hz), 7.07 (1H, t, J = 10.1 Hz), 7.40-7.45 (1H, m), 7.46 (1H, s), 7.61 (1H, s), 7.80 (1H, s), 7.93-7.99 (1H, m), 8.14 (1H, s), 9.84 (1H, s). |

TABLE 10

| | | |
|---|---|---|
| I- 072 | 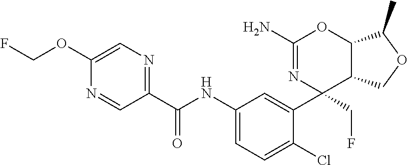 [SMILES: C[C@H]1OC[C@H]2[C@@H]1OC(N)=N[C@]2(CF)c1cc(NC(=O)c2cnc(OCF)cn2)ccc1Cl] | ¹H NMR (400 MHz, CDCl₃) δ: 1.39 (3H, s), 3.31-3.37 (1H, m), 3.91-3.99 (2H, m), 3.93 (3H, s), 4.32-4.45 (5H, m), 5.07 (1H, d, J = 9.3 Hz), 7.07 (1H, t, J = 10.1 Hz), 7.40-7.45 (1H, m), 7.46 (1H, s), 7.61 (1H, s), 7.80 (1H, s), 7.93-7.99 (1H, m), 8.14 (1H, s), 9.84 (1H, s). |
| I- 080 | 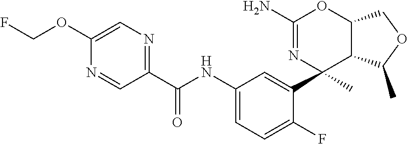 [SMILES: C[C@@H]1OC[C@H]2OC(N)=N[C@](C)(c3cc(NC(=O)c4cnc(OCF)cn4)ccc3F)[C@@H]12] | ¹H NMR (400 MHz, CDCl₃) δ: 1.48 (3H, d, J = 6.0 Hz), 1.57 (3H, s), 2.73 (1H, dd, J = 8.8, 4.4 Hz), 3.85-3.86 (2H, m), 4.20-4.23 (1H, m), 4.25-4.28 (1H, br m), 6.09 (1H, d, J = 3.0 Hz), 6.21 (1H, d, J = 3.0 Hz), 7.07-7.10 (1H, m), 7.39 (1H, dd, J = 7.1, 2.8 Hz), 7.91-7.98 (1H, m), 8.29 (1H, s), 9.08 (1H, s), 9.48 (1H, s). |

TABLE 11

| | | |
|---|---|---|
| I-081 | 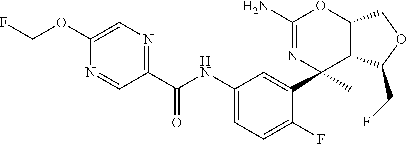 [SMILES: C[C@]1(c2cc(NC(=O)c3cnc(OCF)cn3)ccc2F)N=C(N)O[C@@H]2CO[C@@H](CF)[C@@H]21] | ¹H NMR (400 MHz, CDCl₃) δ: 1.58 (3H, s), 3.19 (1H, dd, J = 8.8, 4.5 Hz), 3.84 (1H, d, J = 10.3 Hz), 3.95 (1H, d, J = 10.3 Hz), 4.25-4.27 (2H, br m), 4.33-4.36 (2H, br m), 4.56 (1H, ddd, J = 47.0, 10.2, 4.0 Hz), 4.75 (1H, ddd, J = 47.0, 10.2, 4.0 Hz), 6.09 (1H, d, J = 3.0 Hz), 6.22 (1H, d, J = 3.0 Hz), 7.08-7.11 (1H, m), 7.44 (1H, dd, J = 7.0, 2.8 Hz), 7.91-7.92 (1H, m), 8.29 (1H, s), 9.08 (1H, s), 9.49 (1H, s). |
| I-082 | 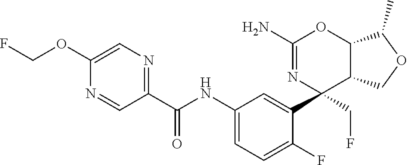 [SMILES: C[C@@H]1OC[C@H]2[C@@H]1OC(N)=N[C@]2(CF)c1cc(NC(=O)c2cnc(OCF)cn2)ccc1F] | ¹H NMR (400 MHz, CDCl₃) δ: 1.39 (3H, s), 3.31-3.37 (1H, m), 3.91-3.99 (2H, m), 3.93 (3H, s), 4.32-4.45 (5H, m), 5.07 (1H, d, J = 9.3 Hz), 7.07 (1H, t, J = 10.1 Hz), 7.40-7.45 (1H, m), 7.46 (1H, s), 7.61 (1H, s), 7.80 (1H, s), 7.93-7.99 (1H, m), 8.14 (1H, s), 9.84 (1H, s). |

TABLE 12

| | | |
|---|---|---|
| I-083 | 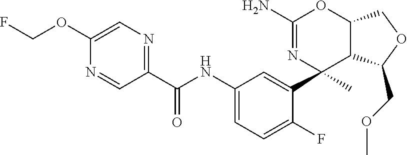 | ¹H NMR (400 MHz, CDCl₃) δ: 1.56 (3H, s), 3.13 (1H, dd, J = 10.3, 5.3 Hz), 3.47 (3H, s), 3.55 (1H, dd, J = 10.3, 5.3 Hz), 3.79 (2H, ddd, J = 22.6, 10.3, 2.0 Hz), 3.92 (1H, d, J = 10.3 Hz), 4.19-4.34 (4H, m), 6.09 (1H, d, J = 2.8 Hz), 6.21 (1H, d, J = 2.8 Hz), 7.07- 7.10 (1H, m), 7.39 (1H, dd, J = 7.0, 2.8 Hz), 7.92-7.96 (1H, m), 8.29 (1H, s), 9.08 (1H, s), 9.48 (1H, s). |
| I-087 | 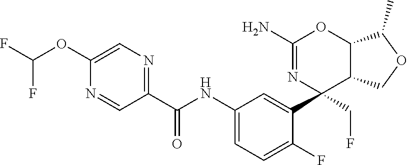 [SMILES: C[C@@H]1OC[C@H]2[C@@H]1OC(N)=N[C@]2(CF)c1cc(NC(=O)c2cnc(OC(F)F)cn2)ccc1F] | ¹H NMR (400 MHz, CDCl₃) δ: 1.25 (3H, d, J = 6.5 Hz), 3.38 (1H, td, J = 9.3, 4.0 Hz), 4.03-4.09 (4H, m), 4.50-4.57 (4H, m), 7.11 (1H, dd, J = 11.7, 8.9 Hz), 7.33-7.69 (2H, m), 7.96 (1H, ddd, J = 8.9, 4.1, 2.8 Hz), 8.33 (1H, d, J = 1.3 Hz), 9.07 (1H, d, J = 1.3 Hz), 9.48 (1H, s). |

TABLE 13

| | | |
|---|---|---|
| I-090 | 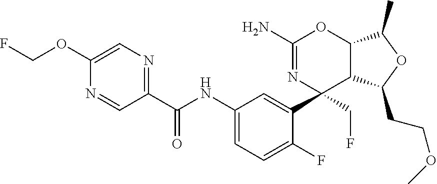 | ¹H NMR (400 MHz, CDCl₃) δ: 1.12 (3H, d, J = 6.8 Hz), 1.77-1.82 (1H, m), 2.14-2.21 (1H, m), 3.03 (1H, dd, J = 9.6, 4.3 Hz), 3.37 (3H, s), 3.54-3.64 (2H, m), 4.05 (1H, d, J = 4.5 Hz), 4.26 (1H, t, J = 9.0 Hz), 4.20-4.58 (2H, br s), 4.60 (1H, s), 4.72 (1H, s), 6.09 (1H, d, J = 5.8 Hz), 6.21 (1H, d, J = 5.8 Hz), 7.10-7.13 (1H, m), 7.49 (1H, dd, J = 7.0, 2.7 Hz), 7.98-8.00 (1H, m), 8.29 (1H, s), 9.08 (1H, s), 9.51 (1H, s). |
| I-091 | 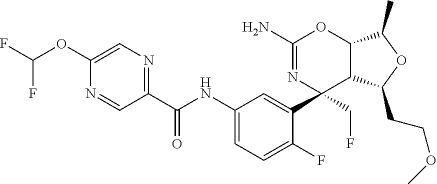 | ¹H NMR (400 MHz, CDCl₃) δ: 1.12 (3H, d, J = 6.7 Hz), 1.79-1.80 (1H, m), 2.15-2.17 (1H, m), 3.03 (1H, dd, J = 9.5, 4.4 Hz), 3.37 (3H, s), 3.57-3.60 (2H, m), 4.04 (1H, d, J = 4.4 Hz), 4.21-4.56 (1H, m), 4.24 (1H, t, J = 9.5 Hz), 4.60 (1H, s), 4.72 (1H, s), 7.11- 7.14 (1H, m), 7.48-7.50 (1H, m), 7.51 (1H, t, J = 82.3 Hz), 7.96-7.99 (1H, m), 8.34 (1H, s), 9.07 (1H, s), 9.49 (1H, s). |

TABLE 14

| | | |
|---|---|---|
| I-092 | 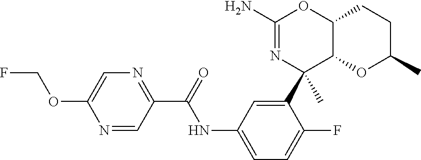 [SMILES: C[C@@H]1CC[C@H]2OC(N)=N[C@](C)(c3cc(NC(=O)c4cnc(OCF)cn4)ccc3F)[C@H]2O1] | ¹H NMR (400 MHz, CDCl₃) δ: 1.24-1.31 (1H, m), 1.36 (3H, d, J = 7.4 Hz), 1.61 (3H, d, J = 1.3 Hz), 1.82-1.86 (2H, m), 2.05-2.14 (1H, m), 3.82 (1H, br s), 4.20 (1H, s), 4.31-4.37 (1H, m), 6.15 (1H, ddd, J = 51.1, 10.8, 1.9 Hz), 7.08 (1H, dd, J = 11.5, 8.8 Hz), 7.38 (1H, dd, J = 6.8, 2.8 Hz), 8.05-8.09 (1H, m), 8.29 (1H, d, J = 1.0 Hz), 9.07 (1H, d, J = 1.0 Hz), 9.53 (1H, br s). |
| I-093 | 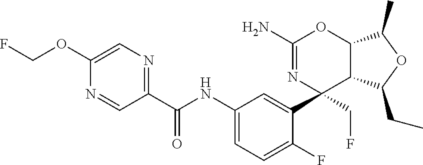 [SMILES: CC[C@@H]1O[C@H](C)[C@H]2OC(N)=N[C@](CF)(c3cc(NC(=O)c4cnc(OCF)cn4)ccc3F)[C@H]21] | ¹H NMR (400 MHz, CDCl₃) δ: 1.07-1.10 (5H, m), 1.56-1.60 (1H, m), 1.90-1.92 (1H, m), 2.94 (1H, dd, J = 9.7, 4.4 Hz), 4.04 (1H, d, J = 4.4 Hz), 4.10-4.12 (2H, m), 4.58 (1H, s), 4.70 (1H, s), 6.08 (1H, d, J = 5.1 Hz), 6.21 (1H, d, J = 5.1 Hz), 7.10-7.13 (1H, m), 7.49 (1H, dd, J = 6.9, 2.8 Hz), 7.98-8.00 (1H, m), 8.27 (1H, s), 9.06 (1H, s), 9.50 (1H, s). |

TABLE 15

| | | |
|---|---|---|
| I-095 | 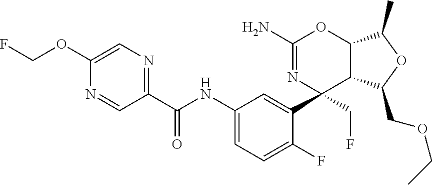 [SMILES: CCOC[C@@H]1O[C@H](C)[C@H]2OC(N)=N[C@](CF)(c3cc(NC(=O)c4cnc(OCF)cn4)ccc3F)[C@H]21] | ¹H NMR (400 MHz, CDCl₃) δ: 1.13 (3H, d, J = 6.8 Hz), 1.26 (3H, t, J = 7.0 Hz), 3.36 (1H, dd, J = 9.6, 4.3 Hz), 3.58-3.63 (3H, m), 3.76 (1H, d, J = 9.6 Hz), 4.08 (1H, d, J = 4.3 Hz), 4.16 (1H, dd, J = 6.8, 4.3 Hz), 4.31-4.34 (1H, br m), 4.60 (1H, dd, J = 26.5, 8.8 Hz), 4.71 (1H, dd, J = 26.5, 8.8 Hz), 6.09 (1H, d, J = 5.0 Hz), 6.21 (1H, d, J = 5.0 Hz), 7.11-7.14 (1H, m), 7.49 (1H, dd, J = 6.8, 2.6 Hz), 7.99- 8.02 (1H, m), 8.30 (1H, s), 9.08 (1H, s), 9.54 (1H, s). |

TABLE 16

| | | |
|---|---|---|
| I-096 | 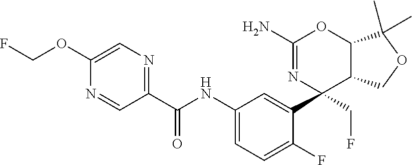 [SMILES: CC1(C)OC[C@H]2[C@@H]1OC(N)=N[C@]2(CF)c1cc(NC(=O)c2cnc(OCF)cn2)ccc1F] | ¹H NMR (400 MHz, CDCl₃) δ: 1.16 (3H, s), 1.28 (3H, s), 3.47-3.52 (1H, m), 3.91 (1H, d, J = 4.4 Hz), 3.97 (1H, t, J = 9.6 Hz), 4.13 (1H, t, J = 9.6 Hz), 4.41 (2H, br), 4.51 (1H, dd, J = 18.0, 8.9 Hz), 4.63 (1H, dd, J = 18.0, 8.9 Hz), 6.15 (2H, dd, J = 51.1, 5.1 Hz), 7.12 (1H, dd, J = 11.6, 8.8 Hz), 7.56 (1H, dd, J = 6.8, 3.5 Hz), 7.96 (1H, dt, J = 8.8, 3.5 Hz), 8.28 (1H, s), 9.07 (1H, s), 9.50 (1H, s). |
| I-097 | 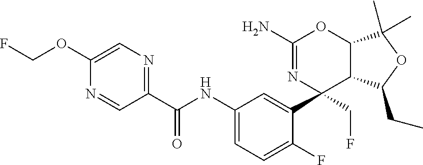 [SMILES: CC[C@@H]1OC(C)(C)[C@H]2OC(N)=N[C@](CF)(c3cc(NC(=O)c4cnc(OCF)cn4)ccc3F)[C@@H]12] | ¹H NMR (400 MHz, CDCl₃) δ: 1.08 (3H, t, J = 7.3 Hz), 1.16 (3H, s), 1.27 (3H, s), 1.50- 1.61 (1H, m), 1.80-1.87 (1H, m), 3.20 (1H, dd, J = 8.9, 4.4 Hz), 3.91 (1H, d, J = 4.4 Hz), 4.15 (1H, t, J = 8.9 Hz), 4.41 (2H, br), 4.56 (1H, dd, J = 25.2, 8.9 Hz), 4.68 (1H, dd, J = 25.2, 8.9 Hz), 6.15 (2H, ddd, J = 51.2, 7.3, 1.9 Hz), 7.12 (1H, dd, J = 11.7, 8.9 Hz), 7.49 (1H, dd, J = 6.9, 3.1 Hz), 7.99 (1H, ddd, J = 8.9, 3.1, 3.1 Hz), 8.29 (1H, s), 9.08 (1H, s), 9.50 (1H, s). |

TABLE 17

| | | |
|---|---|---|
| I-098 | 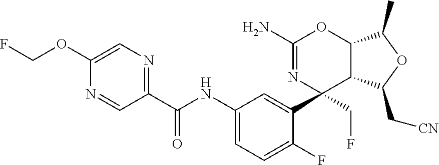 [SMILES: C[C@H]1O[C@@H](CC#N)[C@H]2[C@@H]1OC(N)=N[C@]2(CF)c1cc(NC(=O)c2cnc(OCF)cn2)ccc1F] | ¹H NMR (400 MHz, CDCl₃) δ: 1.21 (3H, d, J = 6.8 Hz), 2.89 (1H, dd, J = 17.3, 3.8 Hz), 3.02 (1H, dd, J = 17.3, 3.8 Hz), 3.26 (1H, dd, J = 9.9, 4.4 Hz), 4.09 (1H, d, J = 4.4 Hz), 4.18 (1H, q, J = 6.8 Hz), 4.32-4.46 (4H, m), 4.92 (1H, dd, J = 46.7, 8.5 Hz), 6.09 (1H, d, J = 3.1 Hz), 6.22 (1H, d, J = 3.1 Hz), 7.11-7.17 (1H, m), 7.61 (1H, dd, J = 6.8, 2.6 Hz), 7.89-7.96 (1H, m), 8.30 (1H, s), 9.08 (1H, s), 9.51 (1H, s). |

TABLE 18

| | | |
|---|---|---|
| I-111 | 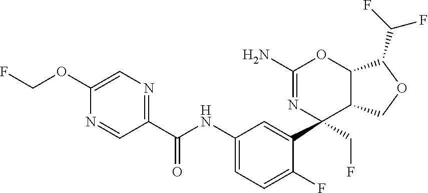 [SMILES: NC1=N[C@](CF)(c2cc(NC(=O)c3cnc(OCF)cn3)ccc2F)[C@H]2CO[C@@H](C(F)F)[C@H]2O1] | ¹H NMR (400 MHz, CDCl₃) δ: 3.33-3.39 (1H, m), 4.04-4.08 (1H, m), 4.11-4.26 (2 H, m), 4.41-4.73 (7 H, m), 6.15 (2H, dd, J = 38.4, 4.8, 3.6 Hz), 7.11 (1H, t, J = 10.2 Hz), 7.60 (1 H, d, J = 6.4 Hz), 7.95 (1H, t, J = 4.3 Hz), 8.27 (1H, s), 9.06 (1H, s), 9.49 (1H, s). |
| I-112 | 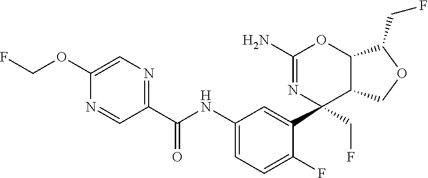 [SMILES: NC1=N[C@](CF)(c2cc(NC(=O)c3cnc(OCF)cn3)ccc2F)[C@H]2CO[C@@H](CF)[C@H]2O1] | ¹H NMR (400 MHz, CDCl₃) δ: 3.34-3.37 (1H, m), 3.67-3.71 (1H, m), 3.99-4.11 (2 H, m), 4.35-4.44 (4 H, m), 4.62-4.73 (1 H, m), 5.87 (1H, t, J = 55.4 Hz), 6.15 (2H, d, J = 50.9 Hz), 7.11-7.14 (1H, m), 7.58- 7.60 (1H, m), 7.95-7.98 (1H, m), 8.29 (1H, d, J = 4.1 Hz), 9.08 (1H, d, J = 4.1 Hz), 9.51 (1H, s). |

TABLE 19

| | | |
|---|---|---|
| I-113 | 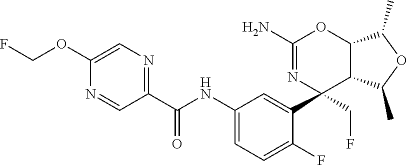 [SMILES: C[C@@H]1O[C@@H](C)[C@H]2[C@@H]1OC(N)=N[C@]2(CF)c1cc(NC(=O)c2cnc(OCF)cn2)ccc1F] | ¹H NMR (400 MHz, CDCl₃) δ: 1.23 (3H, d, J = 6.3 Hz), 1.43 (3H, d, J = 6.0 Hz), 2.95 (1H, dd, J = 8.3, 4.2 Hz), 4.03-4.10 (2H, m), 4.36-4.43 (1 H, m), 4.49 (1H, br s), 4.56 (1H, dd, J = 17.2, 8.9 Hz), 4.68 (1H, dd, J = 17.2, 8.9 Hz), 6.14 (1H, dd, J = 51.2, 1.9 Hz), 6.16 (1H, dd, J = 51.2, 1.9 Hz), 7.09 (1H, dd, J = 11.6, 8.8 Hz), 7.51 (1H, dd, J = 6.9, 2.8 Hz), 7.96-8.00 (1H, m), 8.27 (1H, d, J = 1.0 Hz), 9.06 (1 H, d, J = 1.0 Hz), 9.49 (1H, hr s). |

TABLE 20

| | | |
|---|---|---|
| I-114 | 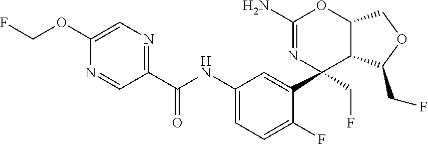 [SMILES: NC1=N[C@](CF)(c2cc(NC(=O)c3cnc(OCF)cn3)ccc2F)[C@H]2[C@H](CF)OC[C@H]2O1] | ¹H NMR (400 MHz, CDCl₃) δ: 3.37 (1H, dd, J = 8.9, 4.4 Hz), 3.83 (1H, d, J = 9.3 Hz), 3.94 (1H, d, J = 9.3 Hz), 4.39-4.76 (7H, m), 4.82 (1H, dd, J = 16.1, 8.6 Hz), 6.09 (1H, dd, J = 5.5, 2.1 Hz), 6.22 (1H, dd, J = 5.5, 2.1 Hz), 7.12 (1H, dd, J = 11.5, 7.8 Hz), 7.56 (1H, dd, J = 7.8, 2.8 Hz), 7.95-7.99 (1H, m), 8.29 (1H, d, J = 1.3 Hz), 9.08 (1H, d, J = 1.3 Hz), 9.51 (1H, s). |

TABLE 21

| | | |
|---|---|---|
| I-115 | 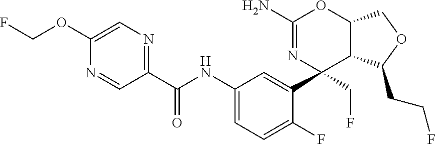 [SMILES: NC1=N[C@](CF)(c2cc(NC(=O)c3cnc(OCF)cn3)ccc2F)[C@H]2[C@H](CCF)OC[C@H]2O1] | ¹H NMR (400 MHz, CDCl₃) δ: 1.89-2.02 (1H, m), 2.18-2.31 (1 H, m), 3.03 (1H, dd, J = 8.7, 4.2 Hz), 3.81 (1H, d, J = 10.3 Hz), 3.91 (1H, d, J = 10.3 Hz), 4.35-4.40 (4H, m), 4.49-4.68 (2 H, m), 4.57-4.79 (2H, m), 6.09 (1H, dd, J = 7.0, 2.0 Hz), 6.22 (1H, dd, J = 7.0, 2.0 Hz), 7.11 (1H, dd, J = 11.6, 8.6 Hz), 7.51 (1H, dd, J = 8.6, 2.8 Hz), 7.96-8.02 (1H, m), 8.29 (1H, d, J = 1.4 Hz), 9.08 (1H, d, J = 1.4 Hz), 9.51 (1 H, s). |

TABLE 22

| | | |
|---|---|---|
| I-116 | 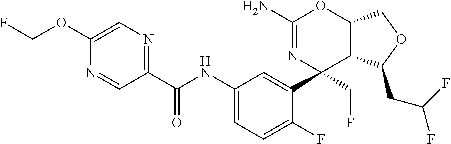 [SMILES: NC1=N[C@](CF)(c2cc(NC(=O)c3cnc(OCF)cn3)ccc2F)[C@H]2[C@H](CC(F)F)OC[C@H]2O1] | ¹H NMR (400 MHz, CDCl₃) δ: 2.02-2.18 (1H, m), 2.30-2.42 (1H, m), 3.02 (1H, dd, J = 9.0, 4.3 Hz), 3.83 (1H, d, J = 10.3 Hz), 3.92 (1H, d, J = 10.3 Hz), 4.37-4.47 (4 H, m), 4.43-4.57 (1H, m), 4.62-4.80 (1H, m), 5.88-6.20 (1H, m), 6.09 (1H, d, J = 4.8 Hz), 6.22 (1H, d, J = 4.8 Hz), 7.11 (1H, dd, J = 11.7, 8.9 Hz), 7.54 (1H, dd, J = 11.7, 8.9 Hz), 7.96-8.01 (1H, m), 8.29 (1H, d, J = 1.4 Hz), 9.08 (1H, d, J = 1.4 Hz), 9.51 (1H, s). |

TABLE 23

| | | |
|---|---|---|
| I-117 | 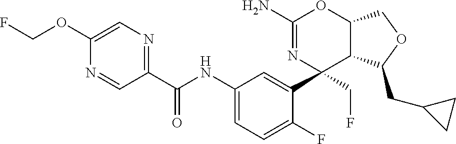 [SMILES: NC1=N[C@](CF)(c2cc(NC(=O)c3cnc(OCF)cn3)ccc2F)[C@H]2[C@H](CC3CC3)OC[C@H]2O1] | ¹H NMR (400 MHz, CDCl₃) δ: 0.10-0.23 (2H, m), 0.45-0.60 (2H, m), 0.90-0.98 (1H, m), 1.40-1.48 (1 H, m), 1.69-1.78 (1H, m), 3.02 (1 H, dd, J = 8.6, 4.3 Hz), 3.79 (1 H, d, J = 10.3 Hz), 3.91 (1H, d, J = 10.3 Hz), 4.28-4.42 (4H, m), 4.53 (1H, dd, J = 14.5, 8.8 Hz), 4.65 (1H, dd, J = 14.5, 8.8 Hz), 6.09 (1H, d, J = 4.8 Hz), 6.22 (1 H, d, J = 4.8 Hz), 7.10 (1H, dd, J = 11.6, 7.8 Hz), 7.48 (1H, dd, J = 7.8, 2.7 Hz), 7.98-8.04 (1H, m), 8.29 (1H, d, J = 1.3 Hz), 9.08 (1H, d, J = 1.3 Hz), 9.51 (1H, s). |

TABLE 24

| | M + H | tR | LC/MS |
|---|---|---|---|
| No | Observed | (min) | method |
| I-016 | 464 | 1.27 | B |
| I-017 | 488 | 1.28 | B |
| I-018 | 460 | 1.08 | B |
| I-019 | 484 | 1.13 | B |
| I-020 | 442 | 1.04 | B |
| I-021 | 466 | 1.06 | B |
| I-022 | 470 | 1.11 | B |
| I-023 | 448 | 1.14 | B |
| I-024 | 480 | 1.62 | B |
| I-025 | 504 | 1.64 | B |
| I-027 | 452 | 1.39 | B |
| I-028 | 476 | 1.41 | B |

TABLE 25

| | | | |
|---|---|---|---|
| I-029 | 504 | 1.6 | B |
| I-030 | 516 | 1.47 | B |
| I-031 | 515 | 1.22 | B |
| I-051 | 471 | 1.02 | B |
| I-070 | 470 | 1.25 | B |

TABLE 26

| | | | |
|---|---|---|---|
| I-072 | 468 | 1.27 | B |
| I-080 | 434 | 1.14 | B |
| I-081 | 452 | 1.13 | B |
| I-082 | 452 | 0.95 | B |
| I-083 | 464 | 1.13 | B |
| I-087 | 470 | 1.02 | B |
| I-090 | 510 | 1.26 | B |
| I-091 | 528 | 1.37 | B |
| I-092 | 448 | 1.4 | B |
| I-093 | 480 | 1.39 | B |
| I-095 | 510 | 1.4 | B |

TABLE 27

| | | | |
|---|---|---|---|
| I-096 | 466 | 1.07 | B |
| I-097 | 494 | 1.28 | B |
| I-098 | 491 | 1.03 | B |
| I-111 | 470 | 0.90 | B |
| I-112 | 488 | 1.11 | B |
| I-113 | 466 | 1.07 | B |
| I-114 | 470 | 1.10 | B |
| I-115 | 484 | 1.13 | B |
| I-116 | 502 | 1.14 | B |
| I-117 | 492 | 1.28 | B |

Further examples are described bellow.

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW) and/or exact mass monoisotopic molecular weight. Data acquisition was performed with appropriate software.

Compounds are described by their experimental retention times (Rₜ) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, etc. . . . ). For molecules with multiple isotopic patterns (Br, Cl.), the reported value is the one obtained for the lowest isotope mass. All results were obtained with experimental uncertainties that are commonly associated with the method used. Hereinafter, “DAD” means Diode Array Detector, “SQD” Single Quadrupole Detector. Table: LCMS Method codes (Flow expressed in mL/min; column temperature (T) in ° C.; Run time in minutes).

TABLE 28

| Method code | Instrument | column | mobile phase | gradient | $\begin{matrix}Flow \\ Col T\end{matrix}$ | Run time |
|---|---|---|---|---|---|---|
| 1 | Waters: Acquity ® IClass UPLC ®- DAD and Xevo G2-S QTOF | Waters: BEH C18 (1.7 μm, 2.1 × 50 mm) | A: 95% CH₃COONH₄ 6.5 mM + 5% CH₃CN, B: CH₃CN | From 95% A to 5% A in 4.6 min, held for 0.4 min | $\begin{matrix}1 \\ 50\end{matrix}$ | 5 |
| 2 | Waters: Acquity ® UPLC ®- DAD and SQD | Waters: BEH C18 (1.7 μm, 2.1 × 50 mm) | A: 95% CH₃COONH₄ 6.5 mM + 5% CH₃CN, B: CH₃CN | From 95% A to 5% A in 2.0 min, held for 0.5 min | $\begin{matrix}0.8 \\ 50\end{matrix}$ | 2.5 |
| 3 | Waters: Acquity ® IClass UPLC ®- DAD and SQD | Agilent: RRHD (1.8 μm, 2.1 × 50 mm) | A: 95% CH₃COONH₄ 6.5 mM + 5% CH₃CN, B: CH₃CN | From 95% A to 5% A in 2.0 min, held for 0.5 min | $\begin{matrix}0.8 \\ 50\end{matrix}$ | 2.5 |
| 4 | Waters: Acquity UPLC ®-DAD and Quattro Micro ™ | Waters: BEH C18 (1.7 μm, 2.1 × 100 mm) | A: 95% CH₃COONH₄ 7 mM/5% CH₃CN, B: CH₃CN | 84.2% A for 0.49 min, to 10.5% A in 2.18 min, held for 1.94 min, back to 84.2% A in 0.73 min, held for 0.73 min. | $\begin{matrix}0.343 \\ 40\end{matrix}$ | 6.2 |
| 5 | Waters: Acquity ® IClass UPLC ®- DAD and Xevo G2-S QTOF | Waters: BEH C18 (1.7 μm, 2.1 × 50 mm) | A: 95% CH₃COONH₄ 6.5 mM + 5% CH₃CN, B: CH₃CN | From 95% A to 40% A in 1.2 min, to 5% A in 0.6 min, held for 0.2 min | $\begin{matrix}1 \\ 50\end{matrix}$ | 2 |

The GC measurement was performed using a Gas Chromatography system, as specified in the respective methods, coupled to a Mass Spectrometer (MS). The MS detector was configured with an electronic impact ionization source/chemical ionization source (EI/CI). It is within the knowledge of the skilled person to set the tune parameters (e.g. scanning range, MS temperatures . . . ) in order to obtain ions allowing the identification of the compound's nominal monoisotopic molecular weight (MW). Data acquisition was performed with appropriate software.

Compounds are described by their experimental retention times (Rₜ) and ions. For EI, if not specified differently in the table of data, the reported molecular ion corresponds to the [M]⁺ (molecular ion). For CI, if not specified differently in the table of data, the reported molecular ion corresponds to the [M+H]⁺ (protonated molecular ion) and in case the compound was not directly ionizable, the type of adduct is specified (i.e. [M+CH5]⁺, etc. . . . ). For molecules with multiple isotopic patterns (Br, Cl.), the reported value is the one obtained for the lowest isotope mass. All results were obtained with experimental uncertainties that are commonly associated with the method used.

Following table shows GCMS method. In the table, Flow expressed in mL/min; Run time in minute.

TABLE 29

| Method code | Instrument | Ionization mode | Column | Oven program | $\begin{matrix}\begin{matrix}Carrier \\ gas \text{/} Iny . \\ mode\end{matrix} \\ Flow\end{matrix}$ | Run time |
|---|---|---|---|---|---|---|
| 6 | Agilent: GC6890- MSD5973N | EI | Agilent: J&W HP- 5MS column (0.18 μm, 0.18 mm × 20 m) | 50° C., hold for 0.8 min, then 60° C./min for 4.17 min until 300° C., hold for 3.0 min | $\begin{matrix}\begin{matrix}He \text{/} Split , \\ 1 \text{:} 50\end{matrix} \\ 0.7\end{matrix}$ | 8 |

### Example 10

### Synthesis of Compound II-016

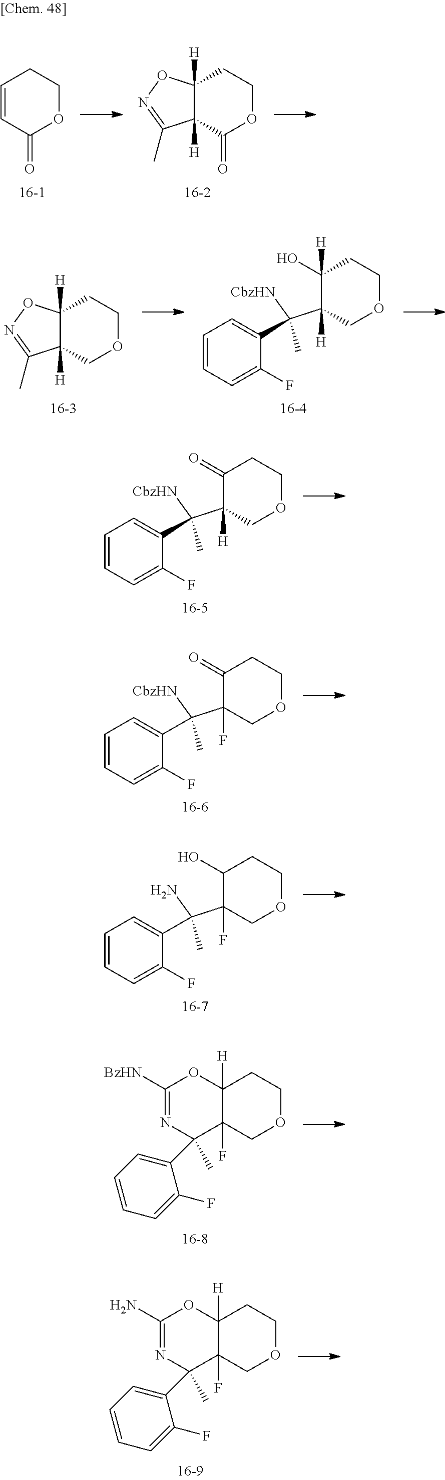

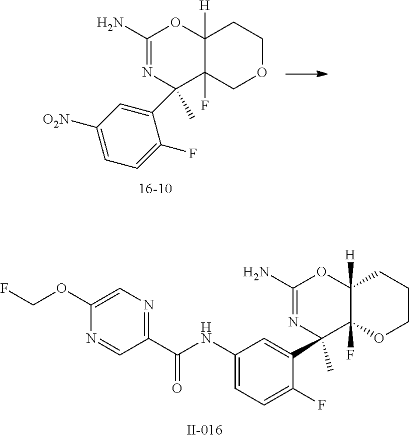

### Step 1

A mixture of 16-1 (3.00 g, 29.1 mmol), and phenyl isocycnate (9.47 mL, 87.2 mmol), nitroethane (3.15 mL, 43.6 mmol), and DIPEA (0.501 mL, 2.91 mmol) in toluene (90 mL) was stirred at 70° C. for 5 hours under N₂. After the mixture was cooled to rt, the resulting slurry was filtered. The solid was washed with toluene and the filtrate was evaporated. The crude product was added to a silica gel column and eluted with n-heptane/EtOAc 100/0 to 70/30. Collected fractions were evaporated to afford Compound 16-2 (4.36 g, 28.1 mmol, 97%) as a yellow oil.

GC/MS: Method 6, M=155, tR=3.71 min.

### Step 2

To a mixture of 16-2 (4.36 g, 28.1 mmol) was added dropwise DIBAL (1 mol/L; 30.9 mL, 30.9 mmol) in DCM (49 mL) at −78° C. under N₂. The mixture was stirred at −78° C. for 30 minutes. The reaction was quenched with MeOH (5.2 mL), followed by sulfuric acid (3.6 mol/L; 35 mL) below −30° C. The resulting solution was stirred at room temperature. The organic layer was separated, dried over sodium sulfate, and filtered. The solvent was evaporated to afford a crude product (3.94 g), which was used as such for the next step without further purification.

To a mixture of the crude product (7.60 g) in DCM (85 mL) and acetnitrile (85 mL) were added triethylsilane (23.2 mL, 145 mmol) followed by BF₃-OEt₂ (17.9 mL, 145 mmol) at −78° C. under N₂. The reaction was stirred at 0° C. for 45 minutes. The reaction was quenched with NaHCO₃ aq. The organic layer was separated, dried over sodium sulfate, and filtered. The solvent was evaporated. The crude product was added to a silica gel column and eluted with n-heptane/EtOAc 60/40 to 0/100. Collected fractions were evaporated to afford Compound 16-3 (4.91 g, 34.8 mmol, 72% in 2 steps) as a yellow oil.

GC/MS: Method 6, M=139, tR=3.42 min.

### Step 3

To a mixture of 16-3 (4.91 g, 34.8 mmol) was added dropwise BuLi (2.5 mol/L; 34.8 mL, 87.0 mmol) in 2-Me THF (81 mL) at −78° C. under N₂. The mixture was stirred at −78° C. for 10 minutes. BF₃-OEt₂ (17.9 mL, 145 mmol) was added dropwise. After the mixture was stirred at the same temperature for 10 minutes, 16-3 (4.91 g, 34.8 mmol) in 2-Me THF (30 mL) was added dropwise at −78° C. The mixture was stirred at −78° C. for 10 minutes and then allowed to warm to 0° C. The reaction was quenched with water (5.2 mL). The organic layer was separated. The aqueous layer was extracted with EtOAc. The combined organic layers were dried over sodium sulfate, and filtered. The solvent was evaporated to afford a crude product (14.5 g), which was used as such for the next step without further purification.

The crude product (14.5 g), Pd/C (1.64 g) in EtOH (102 mL) was stirred at room temperature for 18 hours under H₂. The reaction was diluted with EtOH. The mixture was filtered through a Celite (Registered trademark) pad and washed with EtOH. The filtrate was evaporated to afford a crude product (3.72 g), which was used as such for the next step without further purification.

To a mixture of the crude product (3.72 g), Na₂CO₃ (3.30 g, 31.1 mmol) in THF (164 mL) and H₂O (20 mL) was added benzoyl chloroformate (4.44 mL, 31.1 mmol) at 0° C. The mixture was stirred at room temperature overnight. The mixture was diluted with H₂O. The organic layer was separated. The aqueous layer was extracted with EtOAc. The combined organic layers were dried over sodium sulfate, and filtered. The filtrate was evaporated. The crude product was added to a silica gel column and eluted with DCM/EtOAc 100/0 to 60/40. Collected fractions were evaporated to afford Compound 16-4 (4.50 g, 12.1 mmol, 35% in 3 steps) as a colorless oil.

LC/MS: Method 2, M+1=374, tR=1.35 min.

### Step 4

A mixture of Compound 16-4 (2.94 g, 7.88 mmol), PCC (10.2 g, 47.3 mmol) in DCM (51 mL) was stirred at room temperature for 6 hours. The reaction was quenched with NaHCO aq. and the organic layer was separated. The aqueous layer was extracted with DCM. The combined organic layers were dried over sodium sulfate, and filtered. The filtrate was evaporated. The crude product was added to a silica gel column and eluted with n-heptane/EtOAc 100/0 to 50/50. Collected fractions were evaporated to afford Compound 16-5 (1.41 g, 3.80 mmol, 48%) as a colorless oil.

LC/MS: Method 2, M+1=372, tR=1.37 min.

### Step 5

A mixture of Compound 16-5 (460 mg, 1.24 mmol), Selectfluor (2.19 g, 6.19 mmol) in acetonitrile (23 mL) was stirred at 70° C. for 34 hours. The reaction was quenched with Na₂CO₃ aq. and the organic layer was separated. The aqueous layer was extracted with DCM. The combined organic layers were dried over sodium sulfate, and filtered. The filtrate was evaporated. The crude product was added to a silica gel column and eluted with n-heptane/EtOAc 100/0 to 40/60. Collected fractions were evaporated to afford Compound 16-6 (155 mg, 0.398 mmol, 32%, distereomeric ratio=7:3) as a colorless oil.

LC/MS: Method 2, M+1=390, tR=1.41, 1.43 min.

### Step 6

To a mixture of Compound 16-6 (120 mg, 0.308 mmol) in MeOH (2.4 mL) was added NaBH₄ (23.3 mg, 0.616 mmol) at room temperature. The mixture was stirred at room temperature for 1 hour. The reaction was diluted with Na₂CO₃ aq. and the organic layer was separated. The aqueous layer was extracted with EtOAc. The combined organic layers were dried over sodium sulfate, and filtered. The solvent was evaporated to afford a crude product (114 mg), which was used as such for the next step without further purification.

The crude product (95.0 mg), Pd/C (25.8 mg) in MeOH (4.9 mL) was stirred at room temperature for 5 hours under H₂. The mixture was filtered through a Celite (Registered trademark) pad. The filtrate was evaporated. The crude product was purified by ion exchange chromatography and eluted with MeOH/ammonia. Collected fractions were evaporated to afford Compound 16-7 (45.0 mg, 0.175 mmol, 72%, 4 diasteremeric mixtures) as a colorless oil.

LC/MS: Method 3, M+1=258, tR=0.67, 0.70, 0.80, 0.87 min.

### Step 7

A mixture of Compound 16-7 (183 mg, 0.715 mmol) and benzoyl isothiocyanate (0.14 mL, 1.07 mmol) in DCM (7.4 mL) was stirred at room temperature for 18 hours. EDC-HCl (206 mg, 1.07 mmol) was added and the mixture was stirred at 45° C. for 6 h. The solvent was evaporated. The crude product was added to a silica gel column and eluted with n-heptane/EtOAc 100/0 to 60/40. Collected fractions were evaporated to afford Compound 16-8 (202 mg, 0.523 mmol, 73%) as a white solid. LC/MS: Method 1, M+1=387, tR=2.27 min.

### Step 8

A mixture of Compound 16-8 (240 mg, 0.621 mmol) and hydrazine hydrate (0.30 mL, 6.21 mmol) in MeOH (4.0 mL) and THF (4.0 mL) was stirred at room temperature for 4.5 hours. The solvent was evaporated. The crude product was added to a silica gel column and eluted with DCM/MeOH 100/0 to 85/15. Collected fractions were evaporated to afford Compound 16-9 (175 mg, 0.620 mmol, 99%) as a white solid. LC/MS: Method 2, M+1=283, tR=0.80, 0.84 min.

### Step 9

To a solution of Compound 16-9 (175 mg, 0.620 mmol) in trifluoroacetic acid (0.95 mL) were added sulfuric acid (0.18 mL, 3.41 mmol) and nitric acid (0.042 mL, 12.4 mmol) at −15° C., and the reaction mixture was stirred for at for 30 minutes-10° C. The reaction mixture was quenched with K₂CO₃ aq. below 0° C. The mixture was diluted with EtOAc and H₂O. The organic layer was separated. The aqueous layer was extracted with EtOAc. The combined organic layers were dried over sodium sulfate, and filtered. The solvent was evaporated. The crude product was added to a silica gel column and eluted with DCM/MeOH 95/5. Collected fractions were evaporated to afford Compound 16-10 (145 mg, 0.443 mmol, 71%, 4 diastereomeric mixtures) as a yellow oil. LC/MS: Method 1, M+1=328, tR=1.20, 1.26, 1.28, 1.34 min.

### Step 10

Compound 16-10 (145 mg, 0.443 mmol), Pd/C (33.0 mg), HCl (6 mol/L; 0.177 mL, 0.886 mmol) in EtOH (4 mL) was stirred at room temperature for 18 hours under H₂. The mixture was filtered through a Celite (Registered trademark) pad and washed with EtOH. The residue was taken up in H₂O and MTBE. The aqueous layer was separated. The aqueous layer was basified with Na₂CO₃ aq. and extracted with EtOAc. The combined organic layers were dried over sodium sulfate, and filtered. The solvent was evaporated to give a crude product (112 mg), which was used as such for the next step without further purification.

A mixture of the crude product (112 mg) and HCl (6M; 0.066 mL, 0.396 mmol) in MeOH (4.7 mL) was stirred at room temperature for 15 minutes. 5-(Fluoromethoxy)pyrazine-2-carboxylic acid (71.3 mg, 0.414 mmol) and EDC-HCl (94.9 mg, 0.494 mmol) were added and the mixture was stirred at room temperature for 30 minutes. The solvent was evaporated and the residue was taken up in EtOAc and Na₂CO₃ aq. The organic layer was separated. The aqueous layer was extracted with EtOAc. The combined organic layers were dried over sodium sulfate, and filtered. The solvent was evaporated. The crude product was added to a silica gel column and eluted with DCM/MeOH (7% ammonia solution) 100/0 to 95/5. Collected fractions were evaporated. The crude product was purified by chiral SFC (stationary phase: Chiralcel OD-H 5 μm 250×21.1 mm, mobile phase: 80% CO₂, 20% MeOH (0.3% iPrNH₂)) to afford a mixture of isomers. The mixture was re-purified by chiral SFC (stationary phase: Chiralcel OJ-H 5 μm 250×20 mm, mobile phase: 85% CO₂, 15% EtOH (0.3% iPrNH₂)) to afford a white solid. The solid was diluted with DCM and washed with water. The organic layer was separated, dried over sodium sulfate, filtered, and evaporated to afford Compound II-016 (8.0 mg, 0.0177 mol).

LC/MS: Method 1, M+1=452, tR=1.48 min.

¹H NMR (CDCl₃) δ: 1.64-1.71 (m, 4H), 2.11 (dddt, J=15.0, 12.4, 5.4, 2.8, 2.8 Hz, 1H), 3.58-3.67 (m, 1H), 3.68-3.83 (m, 2H), 4.12 (brs, 1H), 4.22 (t, J=12.6 Hz, 1H), 6.15 (dq, J=51.2, 2.3 Hz, 2H), 7.08 (dd, J=11.4, 8.8 Hz, 1H), 7.59 (dd, J=6.7, 2.6 Hz, 1H), 7.68-7.74 (m, 1H), 8.29 (d, J=1.2 Hz, 1H), 9.08 (d, J=1.2 Hz, 1H), 9.48 (s, 1H).

### Example 11

### Synthesis of Compound II-017

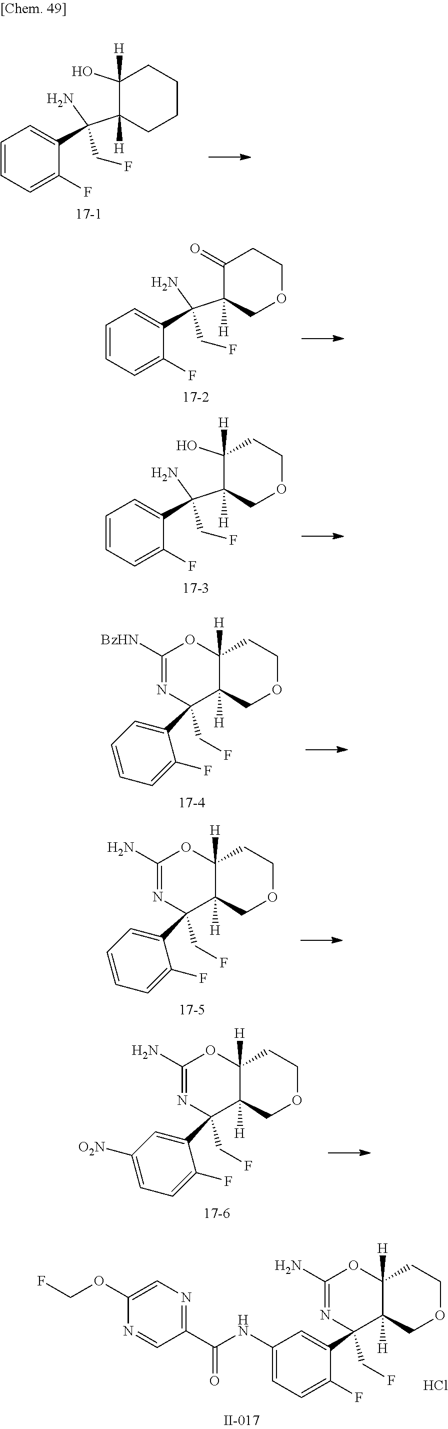

### Step 1

To a mixture of Compound 17-1 (4.0 g, 15.5 mmol) in acetone (120 mL) and water (40 mL) was added chromium trioxide (7.77 g, 77.7 mmol) at 0° C. Then sulfuric acid (11.0 mL, 206 mmol) was added dropwise at 0° C. and the mixture was stirred at 0° C. for 4 hours. The reaction was quenched with Na₂SO₃ aq. and the organic layer was separated. The aqueous layer was extracted with EtOAc. The combined organic layers were dried over sodium sulfate, and filtered. The filtrate was evaporated. The crude product was added to a silica gel column and eluted with n-heptane/EtOAc 70/30 to 40/60. Collected fractions were evaporated to afford Compound 17-2 (3.92 g, 15.4 mmol, 98%) as a colorless oil.

LC/MS: Method 1, M+1=256, tR=1.08, 1.16 min.

### Step 2

To a mixture of tetramethylammonium triacetoxyborohydride (8.25 g, 31.3 mmol) in THF (12 mL) was added acetic acid (4.38 mL, 76.4 mmol) at −25° C. The mixture was stirred at room temperature for 40 minutes and left at 0° C. overnight. Compound 17-2 (1.0 g, 3.92 mmol) in THF (4 mL) was added dropwise to the mixture. The resulting mixture was stirred at −20° C. for 24 hours and left to warm to 0° C. for 2 hours. The reaction was quenched with sodium potassium tartrate aq. and then NaHCO₃ aq. was added at 0° C. The organic layer was separated. The aqueous layer was extracted with EtOAc. The combined organic layers were dried over sodium sulfate, and filtered. The solvent was evaporated. The crude product was purified by HPLC (stationary phase: Xbridge C18 50×100 mm, 5 μm, mobile phase: gradient from 85% NH₄HCO₃ 0.25% solution in water, 15% CH₃CN to 65% NH₄HCO₃ 0.25% solution in water, 35% CH₃CN). Collected fractions were evaporated to afford Compound 17-3 (520 mg, 2.02 mmol, 52%) as a white solid.

LC/MS: Method 5, M+1=258, tR=0.90 min.

### Step 3

A mixture of Compound 17-3 (520 mg, 2.02 mmol) and benzoyl isothiocyanate (0.408 mL, 3.03 mmol) in DCM (21 mL) was stirred at room temperature for overnight. EDC-HCl (581 mg, 3.03 mmol) was added and the mixture was stirred at 45° C. for 6 hours. The solvent was evaporated. The crude product was added to a silica gel column and eluted with n-heptane/EtOAc 100/0 to 60/40. Collected fractions were evaporated to afford Compound 17-4 (630 mg, 1.63 mmol, 81%) as a white solid. LC/MS: Method 1, M+1=387, tR=2.13 min.

### Step 4

A mixture of Compound 17-4 (630 mg, 1.63 mmol) and hydrazine hydrate (0.792 mL, 16.3 mmol) in MeOH (10 mL) and THF (10 mL) was stirred at room temperature for 4.5 hours. The solvent was evaporated. The crude product was added to a silica gel column and eluted with DCM/MeOH 100/0 to 85/15. Collected fractions were evaporated to afford Compound 17-5 (680 mg, 1.20 mmol, 74%) as a white solid. LC/MS: Method 1, M+1=283, tR=0.76 min.

### Step 5

To a solution of Compound 17-5 (680 mg, 1.20 mmol) in trifluoroacetic acid (1.8 mL) were added sulfuric acid (0.35 mL, 6.62 mmol) and nitric acid (0.082 mL, 1.81 mmol) at below −10° C., and the reaction mixture was stirred for at −10° C. for 30 minutes. The reaction mixture was quenched with K₂CO₃ aq. below 0° C. The mixture was diluted with EtOAc and H₂O. The organic layer was separated. The aqueous layer was extracted with EtOAc. The combined organic layers were dried over sodium sulfate, and filtered. The solvent was evaporated. The crude product was added to a silica gel column and eluted with DCM/MeOH 97/3. Collected fractions were evaporated to afford Compound 17-6 (335 mg, 1.02 mmol, 85%) as a yellow oil.

LC/MS: Method 1, M+1=328, tR=0.94 min.

### Step 6

Compound 17-6 (324 mg, 0.990 mmol), Pd/C (73.7 mg), HCl (6 mol/L; 0.396 mL, 1.98 mmol) in EtOH (3.2 mL) was stirred at room temperature for 18 hours under H₂. The mixture was filtered through a Celite (Registered trademark) pad and washed with EtOH. The residue was taken up in H₂O and MTBE. The aqueous layer was separated. The aqueous layer was basified with Na₂CO₃ aq. and extracted with EtOAc. The combined organic layers were dried over sodium sulfate, and filtered. The solvent was evaporated to give a crude product (230 mg), which was used as such for the next step without further purification.

A mixture of the crude product (230 mg) and HCl (6M; 0.135 mL, 0.812 mmol) in MeOH (9.7 mL) was stirred at rt for 15 minutes. 5-(Fluoromethoxy)pyrazine-2-carboxylic acid (146 mg, 0.851 mmol) and EDC-HCl (195 mg, 1.02 mmol) were added and the mixture was stirred at rt for 30 minutes. The solvent was evaporated and the residue was taken up in EtOAc and Na₂CO₃ aq. The organic layer was separated. The aqueous layer was extracted with EtOAc. The combined organic layers were dried over sodium sulfate, and filtered. The solvent was evaporated. The crude product was added to a silica gel column and eluted with DCM/MeOH (7% ammonia solution) 100/0 to 95/5. Collected fractions were evaporated. The product was dissolved in iPrOH (3.5 mL) and warmed to 50° C. HCl (6M; 0.13 mL, 0.774 mmol) and isopropyl ether (7 mL) were added dropwise. The resulting mixture was stirred at rt for 30 minutes. The slurry was filtered and the solid was dried under vacuum to afford Compound II-017 (265 mg, 0.587 mmol, 70%) as a white solid.

LC/MS: Method 1, M+1=452, tR=1.28 min.

¹H NMR (DMSO-d₆) δ: 1.71-1.84 (m, 4H), 2.12 (dd, J=12.1, 4.6 Hz, 1H), 2.41 (td, J=10.8, 4.1 Hz, 1H), 2.55-2.62 (m, 1H), 3.20-3.28 (m, 1H), 3.87 (br dd, J=11.7, 4.8 Hz, 1H), 4.24 (br d, J=10.7 Hz, 1H), 4.44 (td, J=10.8, 4.9 Hz, 1H), 4.94-5.25 (m, 2H), 6.10-6.36 (m, 2H), 7.33 (dd, J=12.4, 9.0 Hz, 1H), 7.93 (dd, J=7.2, 2.6 Hz, 1H), 8.10-8.21 (m, 1H), 8.43 (br s, 1H), 8.60 (d, J=1.5 Hz, 1H), 8.99 (d, J=1.4 Hz, 1H), 9.18 (br s, 1H), 10.44 (br s, 1H), 10.88 (s, 1H).

Test Examples for the compounds of the present invention are mentioned below.

### Pharmacological Examples

The compounds provided in the present invention are inhibitors of the beta-site APP-cleaving enzyme 1 (BACE1). Inhibition of BACE1, an aspartic protease, is believed to be relevant for treatment of Alzheimer's Disease (AD). The production and accumulation of beta-amyloid peptides (Abeta) from the beta-amyloid precursor protein (APP) is believed to play a key role in the onset and progression of AD. Abeta is produced from the amyloid precursor protein (APP) by sequential cleavage at the N- and C-termini of the Abeta domain by BACE1 and gamma-secretase, respectively.

Compounds of Formula (I) are expected to have their effect selectively at BACE1 versus BACE2 by virtue of their ability to selectively bind to BACE1 versus BACE2 and inhibit the BACE1 versus BACE2 enzymatic activity. The behaviour of such inhibitors is tested using a biochemical competitive radioligand binding assay, a biochemical Fluorescence Resonance Energy Transfer (FRET) based assay and a cellular αLisa assay described below, which are suitable for the identification of such compounds.

### Test Example 1: BACE1 and BACE2 Biochemical Competitive Radioligand Binding Assay

To explore the BACE1 versus BACE2 enzyme selectivity, the binding affinity (Ki) to the respective purified enzymes was determined in a competitive radioligand binding assay, i.e. in competition with a tritiated non-selective BACE1/BACE 2 inhibitor.

Briefly in test tubes, compounds of interest were combined with the radioligand and the BACE1 or BACE2-containing HEK 293 derived membrane. The competitive binding reaction was performed at pH 6.2 and incubated at room temperature until the equilibrium was reached. Afterwards free radioligand was separated from bound radioligand by filtration with a Brandell 96 harvester. The filter was washed 4 times with washing buffer and the filter sheets were punched into scintillation vials. Ultima Gold scintillation cocktail was added and samples were shaken. The day after, the vials were counted in a Tricarb scintillation counter to obtain the disintegrations per minute (dpm) of the bound radioligand.

Calculating the % CTL=(sample/HC)*100, with HC being the high control, i.e. total binding of radioligand, allowed to fit curves through the data points of the different doses of test compound. The pIC₅₀ or IC₅₀ was calculated and could be converted to Kᵢ by the formula Kᵢ=IC₅₀/(1+([RL]/K_{d})), with [RL] being the used concentration of radioligand and K_{d} the determinate dissociation constant of the radioligand-membrane complex.

### Test Example 2

### (1) BACE1 Biochemical FRET Based Assay

This assay is a Fluorescence Resonance Energy Transfer Assay (FRET) based assay. The substrate for this assay is an APP derived 13 amino acids peptide that contains the ‘Swedish’ Lys-Met/Asn-Leu mutation of the amyloid precursor protein (APP) beta-site secretase cleavage site. This substrate also contains two fluorophores: (7-methoxycoumarin-4-yl) acetic acid (Mca) is a fluorescent donor with excitation wavelength at 320 nm and emission at 405 nm and 2,4-dinitrophenol (Dnp) is a proprietary quencher acceptor. The distance between those two groups has been selected so that upon light excitation, the donor fluorescence energy is significantly quenched by the acceptor, through resonance energy transfer. Upon cleavage by BACE1, the fluorophore Mca is separated from the quenching group Dnp, restoring the full fluorescence yield of the donor. The increase in fluorescence is linearly related to the rate of proteolysis.

Briefly in a 384-well format recombinant BACE1 protein in a final concentration of 0.04 μg/mL was incubated for 450 minutes at room temperature with 10 μm substrate in incubation buffer (final concentrations: 33.3 mM Citrate buffer pH 5.0, 0.033% PEG, 3% DMSO) in the absence or presence of compound. Next the amount of proteolysis was directly measured by fluorescence measurement at T=0′-120′ and T=450′ (excitation at 320 nm and emission at 405 nm). Results were expressed in RFU (Relative Fluorescence Units), as difference between T450 and Tx (Tx is chosen depending on the reaction speed between 0 and 120 minutes).

A best-fit curve was fitted by a minimum sum of squares method to the plot of % Controlmin versus compound concentration. From this an IC₅₀ value (inhibitory concentration causing 50% inhibition of activity) can be obtained.

$$\begin{matrix}LC = Median of the low control values \\ = Low control \text{:} Reaction without enzyme\end{matrix}$$

$$\begin{matrix}HC = Median of the high control values \\ = High Control \text{:} Reaction with enzyme\end{matrix}$$

$$\% Effect = 100 - [ ( sample - LC ) \text{/} {( H C - L C )}^{*} 1 \; 00 ]$$

$$\% Control = ( sample \text{/} HC ) * 100$$

$$\% Controlmin = ( sample - LC ) \text{/} ( HC - LC ) * 100$$

A compound of the formula (I) has BACE1 inhibiting activity, and it is sufficient that the compound can inhibit the BACE1 receptor.

Specifically, by the protocol above shown, IC50 is preferably 5000 nM or less, more preferably 1000 nM or less, further preferably 100 nM or less.

### (2) BACE2 Biochemical FRET Based Assay

This assay is a Fluorescence Resonance Energy Transfer Assay (FRET) based assay. The substrate for this assay contains the ‘Swedish’ Lys-Met/Asn-Leu mutation of the amyloid precursor protein (APP) beta-secretase cleavage site. This substrate also contains two fluorophores: (7-methoxycoumarin-4-yl) acetic acid (Mca) is a fluorescent donor with excitation wavelength at 320 nm and emission at 405 nm and 2,4-dinitrophenol (Dnp) is a proprietary quencher acceptor. The distance between those two groups has been selected so that upon light excitation, the donor fluorescence energy is significantly quenched by the acceptor, through resonance energy transfer. Upon cleavage by the beta-secretase, the fluorophore Mca is separated from the quenching group Dnp, restoring the full fluorescence yield of the donor. The increase in fluorescence is linearly related to the rate of proteolysis.

Briefly in a 384-well format recombinant BACE2 protein in a final concentration of 0.4 μg/mL was incubated for 450 minutes at room temperature with 10 μM substrate in incubation buffer (final concentrations: 33.3 mM Citrate buffer pH 5.0, 0.033% PEG, 2% DMSO) in the absence or presence of compound. Next the amount of proteolysis was directly measured by fluorescence measurement at T=0 and T=450 (excitation at 320 nm and emission at 405 nm). Results were expressed in RFU (Relative Fluorescence Units), as difference between T450 and T0.

A best-fit curve was fitted by a minimum sum of squares method to the plot of % Controlmin versus compound concentration. From this an IC₅₀ value (inhibitory concentration causing 50% inhibition of activity) can be obtained.

$$\begin{matrix}LC = Median of the low control values \\ = Low control \text{:} Reaction without enzyme\end{matrix}$$

$$\begin{matrix}HC = Median of the high control values \\ = High Control \text{:} Reaction with enzyme\end{matrix}$$

$$\% Effect = 100 - [ ( sample - LC ) \text{/} {( H C - L C )}^{*} 1 \; 00 ]$$

$$\% Control = ( sample \text{/} HC ) * 100$$

$$\% Controlmin = ( sample - LC ) \text{/} ( HC - LC ) * 100$$

The following exemplified compounds were tested essentially as described above and exhibited the following activity:

TABLE 30

| | BACE1 IC50 | BACE2 IC50 | |
|---|---|---|---|
| Compound | (nM) | (nM) | Selectivity |
| I-001 | 19.1 | 339 | 17.8 |
| I-002 | 9.55 | 309 | 32.4 |
| I-003 | 3.80 | 97.7 | 25.7 |
| I-014 | 44.7 | 10000 | 223 |
| I-015 | 14.1 | 955 | 67.7 |
| I-016 | 5.70 | 355 | 62.3 |
| I-017 | 39.8 | 2000 | 50.3 |
| I-018 | 1.20 | 45.7 | 38.1 |
| I-019 | 4.07 | 245 | 60.2 |
| I-020 | 2.19 | 46.8 | 21.4 |
| I-021 | 8.51 | 316 | 37.1 |
| I-022 | 24.0 | 661 | 27.5 |
| I-023 | 4.79 | 245 | 51.2 |
| I-025 | 1.26 | 11.7 | 9.3 |
| I-026 | 6.46 | 240 | 37.2 |
| I-028 | 6.76 | 589 | 87.1 |
| I-029 | 22.9 | 4370 | 191 |
| I-030 | 66.1 | 10000 | 151 |
| I-031 | 12.0 | 3390 | 282 |
| I-051 | 5.01 | 316 | 63.1 |
| I-082 | 11 | 309 | 28 |
| I-116 | 13 | 389 | 31 |
| II-016 | 12.3 | 151 | 12.3 |
| II-017 | 7.6 | 178 | 23.4 |

### Test Example 3-1: Lowering Effect on the Brain β Amyloid in Rats

Compound of the present invention is suspended in 0.5% methylcellulose, the final concentration is adjusted to 2 mg/mL, and this is orally administered to male Crl:SD rat (7 to 9 weeks old) at 10 mg/kg. In a vehicle control group, only 0.5% methylcellulose is administered, and an administration test is performed at 3 to 8 animals per group. A brain is isolated 3 hours after administration, a cerebral hemisphere is isolated, a weight thereof is measured, the hemisphere is rapidly frozen in liquid nitrogen, and stored at −80° C. until extraction date. The frozen cerebral hemisphere is transferred to a homogenizer manufactured by Teflon (Registered trademark) under ice cooling, a 4-fold volume of a weight of an extraction buffer (containing 1% CHAPS ({3-[(3-chloroamidopropyl)dimethylammonio]-1-propanesulfonate}), 20 mmol/L Tris-HCl (pH 8.0), 150 mmol/L NaCl, Complete (Roche) protease inhibitor) is added, up and down movement is repeated, and this is homogenized to solubilize for 2 minutes. The suspension is transferred to a centrifugation tube, allowed to stand on an ice for 3 hours or more and, thereafter centrifuged at 100,000×g, 4° C. for 20 minutes. After centrifugation, the supernatant is transferred to an ELISA plate (product No. 294-62501, Wako Junyaku Kogyo) for measuring β amyloid 40. ELISA measurement is performed according to the attached instruction. The lowering effect is calculated as a ratio compared to the brain β amyloid 40 level of vehicle control group of each test.

### Test Example 3-2: Lowering Effect on the Brain β Amyloid in Mice

Compound of the present invention was dissolved in 20% hydroxyl-beta-cyclodextrin, the final concentration was adjusted to 2 mg/mL, and this was orally administered to male Crl:CD1 (ICR) mouse (6 to 8 weeks old) at 1 to 10 mg/kg. In a vehicle control group, only 20% hydroxyl-beta-cyclodextrin was administered, and an administration test was performed at 3 to 6 animals per group. A brain was isolated 1 to 6 hours after administration, a cerebral hemisphere was isolated, a weight thereof was measured, the hemisphere was rapidly frozen in liquid nitrogen, and stored at −80° C. until extraction date.

The frozen cerebral hemisphere was transferred to a homogenize tube containing ceramic beads in a 8-fold volume of a weight of an extraction buffer (containing 0.4% DEA (diethylamine), 50 mmol/L NaCl, Complete protease inhibitor (Roche)) and incubated on an ice for 20 minutes. Thereafter, the homogenization was done using MP BIO FastPrep (Registered trademark)-24 with Lysing matrix D 1.4 mm ceramic beads (20 seconds at 6 m/s). Then, the tube spins down for 1 minute, the supernatant was transferred to a centrifugation tube, and centrifuged at 221,000×g, 4° C. for 50 minutes. After centrifugation, the supernatant was transferred to Nunc Maxisorp (Registered trademark) plate (Thermo Fisher Scientific) coating with antibody against N-terminal of β amyloid for measuring total β amyloid, and the plate was incubated overnight at 4° C. The plate was washed with TBS-T (Tris buffered saline containing 0.05% Triton X-100), and HRP-conjugated 4G8 dissolved in PBS (pH 7.4) containing 0.1% casein was added in the plate and incubated at 4° C. for 1 hour. After it was washed with TBS-T, SuperSignal ELISA Pico Chemiluminescent Substrate (Thermo Scientific) was added in the plate. Then, the chemi-luminescence counting was measured by ARVO (Registered trademark) MX 1420 Multilabel Counter (Perkin Elmer) as soon as possible. The lowering effect was calculated as a ratio compared to the brain total β amyloid level of vehicle control group of each test.

### Test Example 4-1: CYP3A4 Fluorescent MBI Test

The CYP3A4 fluorescent MBI test is a test of investigating enhancement of CYP3A4 inhibition of a compound by a metabolism reaction. 7-benzyloxytrifluoromethylcoumarin (7-BFC) is debenzylated by the CYP3A4 enzyme (enzyme expressed in Escherichia coli) and 7-hydroxytrifluoromethylcoumarin (7-HFC) is produced as a fluorescing metabolite. The test is performed using 7-HFC production reaction as a marker reaction.

The reaction conditions are as follows: substrate, 5.6 μmol/L 7-BFC; pre-reaction time, 0 or 30 minutes; substrate reaction time, 15 minutes; reaction temperature, 25° C. (room temperature); CYP3A4 content (expressed in Escherichia coli), 62.5 pmol/mL at pre-reaction time, 6.25 pmol/mL (10-fold dilution) at reaction time; concentrations of the compound of the present invention, 0.625, 1.25, 2.5, 5, 10, 20 μmol/L (6 points).

An enzyme in a K-Pi buffer (pH 7.4) and a compound of the present invention solution as a pre-reaction solution are added to a 96-well plate at the composition of the pre-reaction. A part of pre-reaction solution is transferred to another 96-well plate, and diluted 10-fold by a substrate in a K-Pi buffer. NADPH as a co-factor is added in order to initiate a marker reaction (without preincubation). After a predetermined time of the marker reaction, acetonitrile/0.5 mol/L Tris (trishydroxyaminomethane)=4/1 (v/v) solution is added in order to terminate the marker reaction. On the other hand, NADPH is also added to a remaining pre-reaction solution in order to initiate a pre-reaction (with preincubation). After a predetermined time of a pre-reaction, a part is transferred to another 96-well plate, and diluted 10-fold by a substrate in a K-Pi buffer in order to initiate the marker reaction. After a predetermined time of the marker reaction, acetonitrile/0.5 mol/L Tris (trishydroxyaminomethane)=4/1 (v/v) solution is added in order to terminate the marker reaction. Fluorescent values of 7-HFC as a metabolite are measured in each index reaction plate with a fluorescent plate reader (Ex=420 nm, Em=535 nm).

The sample adding DMSO to a reaction system instead of compound of the present invention solution is adopted as a control (100%) because DMSO is used as a solvent to dissolve a compound of the present invention. Remaining activity (%) is calculated at each concentration of the compound of the present invention added as the solution, and IC₅₀ value is calculated by reverse-presumption using a logistic model with a concentration and an inhibition rate. When a difference subtracting IC₅₀ value with preincubation from that without preincubation is 5 μM or more, this is defined as positive (+). When the difference is 3 μM or less, this is defined as negative (−).

### (Test Example 4-2: CYP3A4(MDZ) MBI Test)

CYP3A4(MDZ) MBI test is a test of investigating mechanism based inhibition (MBI) potential on CYP3A4 inhibition of a compound. CYP3A4 inhibition is evaluated using 1-hydroxylation reaction of midazolam (MDZ) by pooled human liver microsomes as a marker reaction.

The reaction conditions were as follows: substrate, 10 μmol/L MDZ; pre-reaction time, 0 or 30 minutes; substrate reaction time, 2 minutes; reaction temperature, 37° C.; protein content of pooled human liver microsomes, 0.5 mg/mL at pre-reaction time, 0.05 pmg/mL (at 10-fold dilution) at reaction time; concentrations of the compound of the present invention, 1, 5, 10, 20 μmol/L (4 points).

Pooled human liver microsomes in a K-Pi buffer (pH 7.4) and a compound of the present invention solution as a pre-reaction solution were added to a 96-well plate at the composition of the pre-reaction. A part of pre-reaction solution was transferred to another 96-well plate, and diluted 10-fold by a substrate in a K-Pi buffer. NADPH as a co-factor was added to initiate the marker reaction (without preincubation). After a predetermined time of the marker reaction, methanol/acetonitrile=1/1 (v/v) solution was added in order to terminate the marker reaction. On the other hand, NADPH was also added to a remaining pre-reaction solution in order to initiate a pre-reaction (with preincubation). After a predetermined time of a pre-reaction, a part was transferred to another 96-well plate, and diluted 10-fold by a substrate in a K-Pi buffer in order to initiate the marker reaction. After a predetermined time of the marker reaction, methanol/acetonitrile=1/1 (v/v) solution is added in order to terminate the marker reaction. After centrifuged at 3000 rpm for 15 minutes, 1-hydroxymidazolam in the supernatant is quantified by LC/MS/MS.

The sample adding DMSO to a reaction system instead of compound of the present invention solution was adopted as a control (100%) because DMSO is used as a solvent to dissolve a compound of the present invention. Remaining activity (%) was calculated at each concentration of the compound of the present invention added as the solution, and IC₅₀ value was calculated by reverse-presumption using a logistic model with a concentration and an inhibition rate. Shifted IC value was calculated as “IC value without preincubation (0 minutes)/IC value with preincubation (30 minutes)”. When a shifted IC value was 1.5 or more, this was defined as positive. When a shifted IC value was less than 1.1, this was defined as negative.

### Test Example 5: CYP Inhibition Test

The CYP inhibition test is a test to assess the inhibitory effect of a compound of the present invention towards typical substrate metabolism reactions on CYP enzymes in human liver microsomes. The marker reactions on human main five CYP enzymes (CYP1A2, 2C9, 2C19, 2D6, and 3A4) were used as follows; 7-ethoxyresorufin 0-deethylation (CYP1A2), tolbutamide methyl-hydroxylation (CYP2C9), mephenytoin 4′-hydroxylation (CYP2C19), dextromethorphan 0-demethylation (CYP2D6), and terfenadine hydroxylation (CYP3A4). The commercially available pooled human liver microsomes were used as an enzyme resource.

The reaction conditions were as follows: substrate, 0.5 μmol/L ethoxyresorufin (CYP1A2), 100 μmol/L tolbutamide (CYP2C9), 50 μmol/L S-mephenytoin (CYP2C19), 5 μmol/L dextromethorphan (CYP2D6), 1 μmol/L terfenadine (CYP3A4); reaction time, 15 minutes; reaction temperature, 37° C.; enzyme, pooled human liver microsomes 0.2 mg protein/mL; concentrations of the compound of the present invention, 1, 5, 10, 20 μmol/L (4 points).

Five kinds of substrates, human liver microsomes, and a compound solution of the present invention in 50 mmol/L Hepes buffer were added to a 96-well plate at the composition as described above as a reaction solution. NADPH as a cofactor was added to this 96-well plate in order to initiate marker reactions. After the incubation at 37° C. for 15 minutes, a methanol/acetonitrile=1/1 (v/v) solution was added in order to terminate the marker reactions. After the centrifugation at 3000 rpm for 15 minutes, resorufin (CYP1A2 metabolite) in the supernatant was quantified by a fluorescent plate reader or LC/MS/MS, and hydroxytolbutamide (CYP2C9 metabolite), 4′-hydroxymephenytoin (CYP2C19 metabolite), dextrorphan (CYP2D6 metabolite), and terfenadine alcohol metabolite (CYP3A4 metabolite) in the supernatant were quantified by LC/MS/MS.

The sample adding DMSO to a reaction system instead of compound of the present invention solution was adopted as a control (100%) because DMSO was used as a solvent to dissolve a compound of the present invention. Remaining activity (%) was calculated at each concentration of a compound of the present invention, and IC₅₀ value was calculated by reverse presumption using a logistic model with a concentration and an inhibition rate.

### Test Example 6: Fluctuation Ames Test

Each 20 μL of freeze-stored Salmonella typhimurium (TA98 and TA100 strain) is inoculated in 10 mL of liquid nutrient medium (2.5% Oxoid nutrient broth No. 2), and the cultures are incubated at 37° C. under shaking for 10 hours. 7.70 to 8.00 mL of TA98 culture is centrifuged (2000×g, 10 minutes) to remove medium, and the bacteria is suspended in 7.70 mL of Micro F buffer (K₂HPO₄: 3.5 g/L, KH₂PO₄: 1 g/L, (NH₄)₂SO₄: 1 g/L, trisodium citrate dihydrate: 0.25 g/L, MgSO₄-7H₂O: 0.1 g/L), and the suspension is added to 120 mL of Exposure medium (Micro F buffer containing Biotin: 8 μg/mL, histidine: 0.2 μg/mL, glucose: 8 mg/mL). 3.10 to 3.42 mL of TA100 culture is added to 130 mL of Exposure medium to prepare the test bacterial solution. 588 μL of the test bacterial solution (or mixed solution of 498 μL of the test bacterial solution and 90 μL of the S9 mix in the case with metabolic activation system) are mixed with each 12 μL of the following solution: DMSO solution of the compound of the present invention (several stage dilution from maximum dose 50 mg/mL at 2 to 3-fold ratio); DMSO as negative control; 50 μg/mL of 4-nitroquinoline-1-oxide DMSO solution as positive control for TA98 without metabolic activation system; 0.25 μg/mL of 2-(2-furyl)-3-(5-nitro-2-furyl)acrylamide DMSO solution as positive control for TA100 without metabolic activation system; 40 μg/mL of 2-aminoanthracene DMSO solution as positive control for TA98 with metabolic activation system; or 20 μg/mL of 2-aminoanthracene DMSO solution as positive control for TA100 with metabolic activation system. A mixed solution is incubated at 37° C. under shaking for 90 minutes. 460 μL of the bacterial solution exposed to the compound of the present invention is mixed with 2300 μL of Indicator medium (Micro F buffer containing biotin: 8 μg/mL, histidine: 0.2 μg/mL, glucose: 8 mg/mL, Bromo Cresol Purple: 37.5 μg/mL), each 50 μL is dispensed into 48 wells/dose in the microwell plates, and is subjected to stationary cultivation at 37° C. for 3 days. A well containing the bacteria, which has obtained the ability of proliferation by mutation in the gene coding amino acid (histidine) synthetase, turns the color from purple to yellow due to pH change. The number of the yellow wells among the 48 total wells per dose is counted, and evaluate the mutagenicity by comparing with the negative control group. (−) means that mutagenicity is negative and (+) means positive.

### Test Example 7: Solubility Test

The solubility of each compound of the present invention was determined under 1% DMSO addition conditions. A 10 mmol/L solution of the compound was prepared with DMSO, and 2 μL of the compound of the present invention solution was added, respectively, to 198 μL of JP 1st fluid (water was added to 2.0 g of sodium chloride and 7.0 mL of hydrochloric acid to reach 1000 mL) and JP 2nd fluid (1 volume of water was added to 1 volume of the solution which 3.40 g of potassium dihydrogen phosphate and 3.55 g of anhydrous disodium hydrogen phosphate dissolve in water to reach 1000 mL). The mixture was left standing for 16 hours at 25° C. or shaken for 1 hour at room temperature, and the mixture was vacuum-filtered. The filtrate was ten or one hundred-fold diluted with methanol/water=1/1 (v/v) or MeCN/MeOH/H₂O (=1/1/2), and the compound concentration in the filtrate was measured with LC/MS or solid phase extraction (SPE)/MS by the absolute calibration method.

### Test Example 8: Metabolic Stability Test

Using a commercially available pooled human liver microsomes, a compound of the present invention was reacted for a constant time, a remaining rate was calculated by comparing a reacted sample and an unreacted sample, thereby, a degree of metabolism in liver was assessed.

A reaction was performed (oxidative reaction) at 37° C. for 0 minute or 30 minutes in the presence of 1 mmol/L NADPH in 0.2 mL of a buffer (50 mmol/L Tris-HCl pH 7.4, 150 mmol/L potassium chloride, 10 mmol/L magnesium chloride) containing 0.5 mg protein/mL of human liver microsomes. After the reaction, 50 μL of the reaction solution was added to 100 μL of a methanol/acetonitrile=1/1 (v/v), mixed and centrifuged at 3000 rpm for 15 minutes. The compound of the present invention in the supernatant was quantified by LC/MS/MS or solid phase extraction (SPE)/MS, and a remaining amount of the compound of the present invention after the reaction was calculated, letting a compound amount at 0 minute reaction time to be 100%.

TABLE 31

| | Remaining rate (%) |
|---|---|
| No. | at 30 min |
| I-001 | 86 |
| I-002 | 78 |
| I-003 | 69 |
| I-017 | 103 |
| I-070 | 52 |
| I-082 | 92 |
| I-087 | 85 |
| I-096 | 65 |
| I-112 | 91 |
| I-114 | 102 |
| I-115 | 93 |
| I-116 | 92 |

### Test Example 9: hERG Test

For the purpose of assessing risk of an electrocardiogram QT interval prolongation, effects on delayed rectifier K+ current (I_{Kr}), which plays an important role in the ventricular repolarization process of the compound of the present invention, was studied using CHO cells expressing human ether-a-go-go related gene (hERG) channel.

A cell was retained at a membrane potential of −80 mV by whole cell patch clamp method using an automated patch clamp system (QPatch; Sophion Bioscience A/S). After application of leak potential at −50 mV, I_{Kr} induced by depolarization pulse stimulation at +20 mV for 2 seconds and, further, repolarization pulse stimulation at −50 mV for 2 seconds was recorded.

After the generated current was stabilized, extracellular solution (NaCl: 145 mmol/L, KCl: 4 mmol/L, CaCl₂:2 mmol/L MgCl₂:1 mmol/L, 1 mmol/L, HEPES (4-(2-hydroxyethyl)-1-piperazineethanesulfonic acid: 10 mmol/L, glucose:10 mmol/L pH=7.4) in which the compound of the present invention have been dissolved at an objective concentration was applied to the cell under the room temperature condition for 10 minutes. From the recording I_{Kr}, an absolute value of the tail peak current was measured based on the current value at the resting membrane potential using an analysis software (QPatch assay software; Sophion Bioscience A/S). Further, the % inhibition relative to the tail peak current before application of the compound of the present invention was calculated, and compared with the vehicle-applied group (0.1% dimethyl sulfoxide solution) to assess influence of the compound of the present invention on I_{Kr}.

The following data show the inhibition at 3 μM of the compounds of the present invention.

TABLE 32

| | hERG inhibition (%) |
|---|---|
| No. | at 3 μM |
| I-001 | 10.4 |
| I-002 | 21.9 |
| I-003 | 8.4 |
| I-017 | 32 |
| I-070 | 9.4 |
| I-082 | 6.7 |
| I-087 | 28.7 |
| I-096 | 19.4 |
| I-112 | 5 |
| I-114 | 12.4 |
| I-115 | 9.9 |
| I-116 | 33.9 |

### Test Example 10: Powder Solubility Test

Appropriate amounts of the compound of the present invention are put into appropriate containers. 200 μL of JP 1^{st} fluid (water is added to 2.0 g of sodium chloride and 7.0 mL of hydrochloric acid to reach 1000 mL), 200 μL of JP 2^{nd} fluid (1 volume of water is added to 1 volume of the solution which 3.40 g of potassium dihydrogen phosphate and 3.55 g of anhydrous disodium hydrogen phosphate dissolve in water to reach 1000 mL), 200 μL of fasted state simulated intestinal fluid (FaSSIF), and 200 μL of fed state simulated intestinal fluid (FeSSIF) are added to the respective containers. When total amount of the compound of the present invention is dissolved after the addition of the test fluid, the compound is added as appropriate. The containers are sealed, and shaken for 1 and/or 24 hours at 37° C. The mixtures were filtered, and 100 μL of methanol is added to each of the filtrate (100 μL) so that the filtrates are two-fold diluted. The dilution ratio may be changed if necessary. After confirming that there is no bubbles and precipitates in the diluted solution, the containers are sealed and shaken. Quantification is performed by HPLC with an absolute calibration method.

### Test Example 11: Pharmacokinetic Study

Materials and methods for studies on oral absorption

(1) Animal: mouse or rat
(2) Breeding conditions: mouse or rat was allowed free access to the tap water and the solid food.
(3) Dose and grouping: orally or intravenously administered at a predetermined dose; grouping was as follows (Dose depends on the compound)
Oral administration: approximately 1 to 30 mg/kg (n=2 to 3)
Intravenous administration: approximately 0.5 to 10 mg/kg (n=2 to 3)
(4) Dosing formulation: for oral administration, in a solution or a suspension state; for intravenous administration, in a solubilized state
(5) Dosing method: in oral administration, forcedly administer using a syringe attached a flexible feeding tube; in intravenous administration, administer from caudal vein using a syringe attached with a needle.
(6) Evaluation items: blood was collected at the scheduled time, and the plasma concentration of the compound of the present invention was measured by LC/MS/MS
(7) Statistical analysis: regarding the transition of the plasma concentration of the compound of the present invention, the area under the plasma concentration-time curve (AUC) was calculated by trapezoidal method, and the bioavailability (BA) of the compound of the present invention was calculated from the AUCs of the oral administration group and intravenous administration group

### Test Example 12: Brain Distribution Studies

Compound of the present invention was intravenously administered to a rat at approximately 0.5 mg/mL/kg dosage. 30 minutes later, all blood was drawn from the abdominal aorta under isoflurane anesthesia for death from exsanguination.

The brain was enucleated and 20 to 25% of homogenate thereof was prepared with distilled water.

The obtained blood was used as plasma after centrifuging. The control plasma was added to the brain sample at 1:1. The control brain homogenate was added to the plasma sample at 1:1. Each sample was measured using LC/MS/MS. The obtained area ratio (a brain/plasma) was used for the brain Kp value.

### Example 13: Ames Test

Ames test is performed by using Salmonellas (Salmonella typhimurium) TA 98, TA100, TA1535 and TA1537 and Escherichia coli WP2uvrA as test strains with or without metabolic activation in the pre-incubation method to check the presence or absence of gene mutagenicity of compounds of the present invention.

### Test Example 14: P-Gp Substrate Test

1. Cell line:

a. MDR1/LLC-PK1 (Becton Dickinson)

b. LLC-PK1 (Becton Dickinson)

2. Reference substrates:

a. Digoxin (2 μM)

Methods and Procedures

1. MDR1 expressing LLC-PK1 cells and its parent cells were routinely cultured in Medium A (Medium 199 (Invitrogen) supplemented with 10% FBS (Invitrogen), gentamycin (0.05 mg/mL, Invitrogen) and hygromycin B (100 μg/mL, Invitrogen)) at 37° C. under 5% CO_{2/95}% 02 gasses. For the transport experiments, these cells were seeded on Transwell (Registered trademark) insert (96-well, pore size: 0.4 μm, Coaster) at a density of 1.4×10⁴ cells/insert and added Medium B (Medium 199 supplemented with 10% FBS and gentamycin at 0.05 mg/mL) to the feeder tray. These cells were incubated in a CO₂ incubator (5% CO_{2/95}% 02 gasses, 37° C.) and replace apical and basolateral culture medium every 48-72 hr after seeding. These cells were used between 4 and 6 days after seeding.
2. The medium in the culture insert seeded with MDR1 expressing cells or parent cells were removed by aspiration and rinsed by HBSS. The apical side (140 μL) or basolateral side (175 μL) was replaced with transport buffer containing reference substrates and the present invention and then an aliquot (50 μL) of transport buffer in the donor side was collected to estimate initial concentration of reference substrate and the present invention. After incubation for designed time at 37° C., an aliquot (50 μL) of transport buffer in the donor and receiver side were collected. Assay was performed by duplicate or triplicate.
3. Reference substrate and the present invention in the aliquot was quantified by LC/MS/MS.

Calculations

Permeated amounts across monolayers of MDR1 expressing and parent cells were determined, and permeation coefficients (Pe) were calculated using Excel 2003 from the following equitation:

Pe (cm/sec)=Permeated amount (pmol)/area of cell membrane (cm²)/initial concentration (nM)/incubation time (sec)

Where, permeated amount was calculated from permeation concentration (nM, concentration of the receiver side) of the substance after incubation for the defined time (sec) multiplied by volume. (mL) and area of cell membrane was used 0.1433 (cm2). The efflux ratio was calculated using the following equation:

Efflux Ratio=Basolateral-to-Apical Pe/Apical-to-Basolateral Pe

The net flux was calculated using the following equation:

Net flux=Efflux Ratio in MDR1 expressing cells/Efflux Ratio in parent cells

TABLE 33

| No. | P-gp ER ratio |
|---|---|
| I-001 | 3.8 |
| I-002 | 3.4 |
| I-003 | 3.6 |
| I-017 | 3.4 |
| I-070 | 3.8 |
| I-082 | 3.6 |
| I-087 | 5.8 |
| I-096 | 3.3 |
| I-112 | 5.3 |
| I-114 | 2.1 |
| I-115 | 3 |
| I-116 | 5.2 |

### Test Example 15: Inhibitory Effects on P-Gp Transport

Materials

1. Cell line:

a. MDR1/LLC-PK1 (Becton Dickinson)

b. LLC-PK1 (Becton Dickinson)

2. Reference substrates:

a. [³H]Digoxin (1 μM)

b. [¹⁴C]Mannitol (1 μM)

3. Reference inhibitor:

Verapamil (1 μM)

Methods and Procedures

1. MDR1 expressing LLC-PK1 cells and its parent cells are routinely cultured in Medium A (Medium 199 (Invitrogen) supplemented with 10% FBS (Invitrogen), gentamycin (0.05 mg/mL, Invitrogen) and hygromycin B (100 μg/mL, Invitrogen)) at 37° C. under 5% CO_{2/95}% 02 gasses. For the transport experiments, these cells are seeded on Transwell (Registered trademark) insert (96-well, pore size: 0.4 μm, Coaster) at a density of 1.4×10⁴ cells/insert and added Medium B (Medium 199 supplemented with 10% FBS and gentamycin at 0.05 mg/mL) to the feeder tray. These cells are incubated in a CO₂ incubator (5% CO_{2/95}% 02 gasses, 37° C.) and replace apical and basolateral culture medium every 48-72 hr after seeding. These cells are used between 6 and 9 days after seeding.
2. The medium in the culture insert seeded with MDR1 expressing cells or parent cells are removed by aspiration and rinsed by HBSS. The apical side (150 μL) or basolateral side (200 μL) is replaced with transport buffer containing reference substrates with or without the compound of the present invention and then an aliquot (50 μL) of transport buffer in the donor side is collected to estimate initial concentration of reference substrate. After incubation for designed time at 37° C., an aliquot (50 μL) of transport buffer in the donor and receiver side are collected. Assay is performed by triplicate. 3. An aliquot (50 μL) of the transport buffer is mixed with 5 mL of a scintillation cocktail, and the radioactivity is measured using a liquid scintillation counter.

Calculations

Permeated amounts across monolayers of MDR1 expressing and parent cells are determined, and permeation coefficients (Pe) were calculated using Excel 2003 from the following equitation:

Pe (cm/sec)=Permeated amount (pmol)/area of cell membrane (cm²)/initial concentration (nM)/incubation time (sec)

Where, permeated amount was calculated from permeation concentration (nM, concentration of the receiver side) of the substance after incubation for the defined time (sec) multiplied by volume (mL) and area of cell membrane is used 0.33 (cm²). The efflux ratio will be calculated using the following equation:

Efflux Ratio=Basolateral-to-Apical Pe/Apical-to-Basolateral Pe

The net flux is calculated using the following equation:

Net flux=Efflux Ratio in MDR1 expressing cells/Efflux Ratio in parent cells

The percent of control is calculated as the net efflux ratio of reference compounds in the presence of the compound of the present invention to that in the absence of the compound of the present invention.
IC₅₀ values are calculated using the curve-fitting program XLfit.

### (Test Example 16: P-Gp Substrate Test Using mdr1a/1b (−/−) B6 Mice)

Materials

Animal: mdr1a/1b (−/−) B6 mice (KO mouse) or C57BL/6J mice (Wild mouse)

Methods and Procedures

1. Animals may be fed prior to dosing of the compounds of the present invention.
2. The compounds of the present invention are dosed to three animals for each time point and blood and brain samples are removed at selected time points (e.g. 15 min, 30 min, 1 hr, 2 hr, 4 hr, 6 hr, 8 hr, or 24 hr) after dosing. Blood (0.3-0.7 mL) is collected via trunk blood collection with syringe containing anticoagulants (EDTA and heparin). Blood and tissue (e.g. brain) samples are immediately placed on melting ice.
3. Blood samples are centrifuged (1780×g for 10 minutes) for cell removal to obtain plasma. Then, plasma samples are transferred to a clean tube and stored in a −70° C. freezer until analysis.
4. Tissue (e.g. brain) samples are homogenized at a 1:3 ratio of tissue weight to ml of stilled water and transferred to a clean tube and stored in a −70° C. freezer until analysis.
5. Plasma and tissue (e.g. brain) samples are prepared using protein precipitation and analyzed by LC/MS/MS. The analytical method is calibrated by including a standard curve constructed with blank plasma or brain samples and known quantities of analyte. Quality control samples are included to monitor the accuracy and precision of the methodology.
6. Plasma and brain concentration values (ng/mL and ng/g) are introduced into an appropriate mathematical tool used for calculating the pharmacokinetic parameters. A common platform is the WinNonlin (Registered trademark) pharmacokinetic software modeling program.

Calculations

Kp; Tissue to Plasma concentration ratio
Kp ratio=Kp in KO mouse/Kp in Wild mouse

KO/Wild ratio of AUC Tissue/AUC Plasma={AUC Tissue/AUC Plasma (KO mouse)}/{AUC Tissue/AUC Plasma (Wild mouse)}

### Example 17: Anesthetized Guinea Pig Cardiovascular Study

Animal species: Guinea pig (Slc:Hartley, 4-5 weeks old, male), N=4
Study design:
Dosage: 3, 10, and 30 mg/kg (in principle)
(The compounds of the present invention are administered cumulatively)

### Formulation:

Composition of Vehicle; Dimethylacetamide (DMA): Polyethylene glycol 400 (PEG400):
Distilled water (D.W.)=1:7:2 (in principle).
The compounds of the present invention are dissolved with DMA and then added PEG400 and D.W. Finally, 1.5, 5, and 15 mg/mL solutions are prepared.
Dosing route and schedule:
Intravenous infusion for 10 min (2 mL/kg).
0 to 10 min: 3 mg/kg, 30 to 40 min: 10 mg/kg, 60 to 70 min: 30 mg/kg
Vehicle is administered by the same schedule as the above.
Group composition:
Vehicle group and the compound of the present invention group (4 guinea pigs per group).
Evaluation method:
Evaluation items:
Mean blood pressure [mmHg], Heart rate (derived from blood pressure waveform [beats/min]), QTc (ms), and Toxicokinetics.

### Experimental Procedure:

Guinea pigs are anesthetized by urethane (1.4 g/kg, i.p.), and inserted polyethylene tubes into carotid artery (for measuring blood pressure and sampling blood) and jugular vein (for infusion test compounds). Electrodes are attached subcutaneously (Lead 2). Blood pressure, heart rate and electrocardiogram (ECG) are measured using PowerLab (Registered trademark) system (ADInstruments).

### Toxicokinetics:

Approximately 0.3 mL of blood (approximately 120 μL as plasma) is drawn from carotid artery with a syringe containing heparin sodium and cooled with ice immediately at each evaluation point. Plasma samples are obtained by centrifugation (4° C., 10000 rpm, 9300×g, 2 minutes). The procedure for separation of plasma is conducted on ice or at 4° C. The obtained plasma (TK samples) is stored in a deep freezer (set temperature: −80° C.).

Analysis methods: Mean blood pressure and heart rate are averaged a 30-second period at each evaluation time point. ECG parameters (QT interval [ms] and QTc are derived as the average waveform of a 10-second consecutive beats in the evaluation time points. QTc [Fridericia's formula; QTc=QT/(RR)1/3)] is calculated using the PowerLab (Registered trademark) system. The incidence of arrhythmia is visually evaluated for all ECG recordings (from 0.5 hours before dosing to end of experiment) for all four animals.
Evaluation time points:
Before (pre dosing), and 10, 25, 40, 55, 70, and 85 min after the first dosing.
Data analysis of QTc:
Percentage changes (%) in QTc from the pre-dose value are calculated (the pre-dose value is regarded as 100%). Relative QTc is compared with vehicle value at the same evaluation point.

### Test Example 18: Pharmacology in the Beagle Dog

Test compounds were tested to evaluate the effect on the beta-amyloid profile in cerebrospinal fluid (CSF) of dogs after a single dose, in combination with pharmacokinetic (PK) follow up and limited safety evaluation.

In the case of compounds shown below, two or 4 beagle dogs (1 or 2 male, 1 or 2 female) were dosed with vehicle (1 mL/kg of an aqueous solution of 20% cyclodextrin) and 4 beagle dogs (2 males and 2 females) per dose group were dosed with test compound at the doses indicated in the following Table in an aqueous 20% cyclodextrin solution with a concentration in mg/mL identical to the dose given in mg/kg) on an empty stomach.

CSF was taken in conscious animals directly from the lateral ventricle via a cannula which was screwed in the skull and covered with subcutaneous tissue and skin, before and at 4, 8, 25 and 49 hours after dosing. Eight hours after dosing the animals got access to their regular meal for 30 minutes. Blood was taken for PK follow up (0.5, 1, 2, 4, 8, 25 and 49 hours) and serum samples for biochemistry were taken before and at 8 and 25 h after dosing. The CSF samples were used for measurement of Abeta 1-37, Abeta 1-38, Abeta 1-40 and Abeta 1-42. The results are summarized in the following Table below:

TABLE 34

| | | % Decrease | | |
|---|---|---|---|---|
| | % Decrease | in Abeta 1-42 | % Decrease | |
| | in Abeta 1-42 | at 24 h^{(a)} | in Abeta 1-42 | |
| | at 8 h | or 25 h^{(b)} | at 49 h | |
| | post dosing | post dosing | post dosing | |
| | compared to | compared to | compared to | Dose |
| Co. No. | own baseline | own baseline | own baseline | (mg/kg) |
| I-001 | −37 | −68 | −30 | 2.5 |
| I-002 | −57 | NR | NR | 2.5 |
| I-003 | −64 | −33 | NR | 0.31 |
| I-003 | −70 | −31 | NR | 0.63 |
| I-003 | −78 | −46 | NR | 1.25 |
| I-035 | −52 | NR | NR | 2.5 |
| I-035 | −63 | −27 | NR | 5 |
| I-082 | −58 | −41 | NR | 0.63 |

% decrease indicated at 8 h and at last time point at which relevant decrease (>20% decrease) was observed.

### Test Example 19: Dansyl GSH Trapping Test

Dansyl glutathione (glutathione) trapping is a test of investigating reactive metabolites.

The reaction conditions are as follows: substrate, 50 μmol/L the compounds of the present invention; trapping reagent, 0.1 mmol/L dansyl GSH; protein content of pooled human liver microsomes, 1 mg/mL; pre-reaction time, 5 minutes; reaction time, 60 minutes; reaction temperature, 37° C.

Pooled human liver microsomes and a solution of the compound of the present invention in K-Pi buffer (pH 7.4) as a pre-reaction solution are added to a 96-well plate at the composition of the pre-reaction. NADPH as a cofactor is added to initiate a reaction. After a predetermined time of a reaction, a part is transferred to another 96-well plate, and a solution of acetonitrile including 5 mmol/L dithiothreitol was added to stop the reaction. After centrifuged at 3000 rpm for 15 minutes, fluorescence peak area of the dansyl GSH trapped metabolites is quantified by HPLC with fluorescence detection.

### Test Example 20: [¹⁴C]-KCN Trapping Test

[¹⁴C]-potassium cyanide (KCN) trapping is a test of investigating reactive metabolites.

The reaction conditions are as follows: substrate, 10 or 50 μmol/L the compounds of the present invention; trapping reagent, 1 mmol/L [¹⁴C]-KCN (11.7 μCi/tube); protein content of pooled human liver microsomes, 1 mg/mL; pre-reaction time, 5 minutes; reaction time, 60 minutes; reaction temperature, 37° C.

Pooled human liver microsomes and a solution of the compound of the present invention in K-Pi buffer (pH 7.4) as a pre-reaction solution are added to a 96-well plate at the composition of the pre-reaction. NADPH as a cofactor is added to initiate a reaction. After a predetermined time, the metabolic reactions are terminated and [¹⁴C]-KCN trapped metabolites are extracted to 100 μL methanol solutions by spin-column. Radio peak area of the [¹⁴C]-KCN trapped metabolites is quantified by Radio-HPLC system.

### Formulation Examples

The following Formulation Examples are only exemplified and not intended to limit the scope of the present invention.

### Formulation Example 1: Tablet

| | |
|---|---|
| Compound of the present invention | 15 mg |
| Lactose | 15 mg |
| Calcium stearate | 3 mg |

All of the above ingredients except for calcium stearate are uniformly mixed. Then the mixture is crushed, granulated and dried to obtain a suitable size of granules. Then, calcium stearate is added to the granules. Finally, tableting is performed under a compression force.

### Formulation Example 2: Capsules

| | |
|---|---|
| Compound of the present invention | 10 mg |
| Magnesium stearate | 10 mg |
| Lactose | 80 mg |

The above ingredients are mixed uniformly to obtain powders or fine granules, and then the obtained mixture is filled in capsules.

### Formulation Example 3: Granules

| | |
|---|---|
| Compound of the present invention | 30 g |
| Lactose | 265 g |
| Magnesium stearate | 5 g |

After the above ingredients are mixed uniformly, the mixture is compressed. The compressed matters are crushed, granulated and sieved to obtain suitable size of granules.

### Formulation Example 4: Orally Disintegrated Tablets

The compounds of the present invention and crystalline cellulose are mixed, granulated and tablets are made to give orally disintegrated tablets.

### Formulation Example 5: Dry Syrups

The compounds of the present invention and lactose are mixed, crushed, granulated and sieved to give suitable sizes of dry syrups.

### Formulation Example 6: Injections

The compounds of the present invention and phosphate buffer are mixed to give injection.

### Formulation Example 7: Infusions

The compounds of the present invention and phosphate buffer are mixed to give injection.

### Formulation Example 8: Inhalations

The compound of the present invention and lactose are mixed and crushed finely to give inhalations.

### Formulation Example 9: Ointments

The compounds of the present invention and petrolatum are mixed to give ointments.

### Formulation Example 10: Patches

The compounds of the present invention and base such as adhesive plaster or the like are mixed to give patches.

### INDUSTRIAL APPLICABILITY

The compounds of the present invention can be a medicament useful as an agent for treating or preventing a disease induced by production, secretion and/or deposition of amyloid β proteins.

1. A compound of Formula (I): 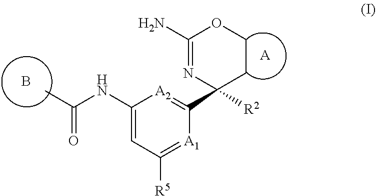
wherein 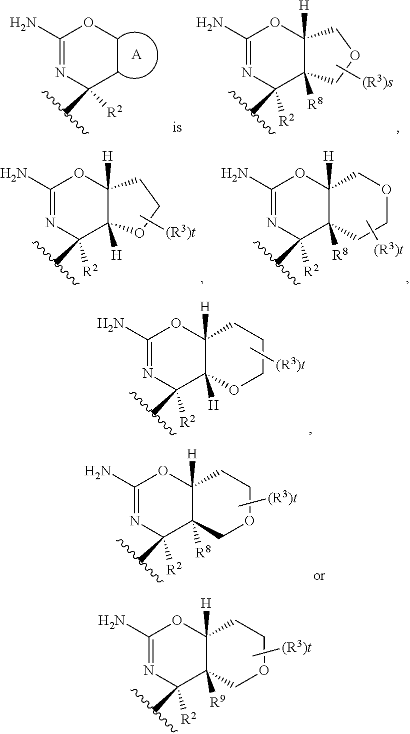
wherein
R² is C1-C3 alkyl optionally substituted with halogen;
R³ is each independently C1-C8 alkyl optionally substituted with one or more group(s) selected from halogen, cyano, C1-C3 alkyloxy, C1-C3 haloalkyloxy, 3- to 6-membered non-aromatic carbocyclyl optionally substituted with halogen, and 3- to 6-membered non-aromatic heterocyclyl optionally substituted with halogen;
aromatic heterocyclyl optionally substituted with one or more group(s) selected from C1-C3 alkyl and C1-C3 haloalkyl; or halogen;
s is an integer from 0 to 3;
t is an integer from 0 to 3;
R⁵ is a hydrogen atom or halogen;
A₁ is CR⁶ or N;
A₂ is CR¹⁰ or N;
R⁶ is a hydrogen atom, halogen, or C1-C3 alkyl optionally substituted with halogen;
R¹⁰ is a hydrogen atom, halogen, or C1-C3 alkyl optionally substituted with halogen; 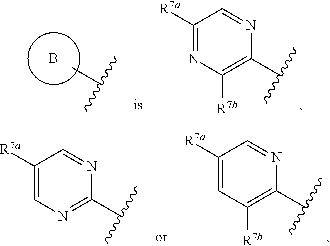
wherein R^{7a} is halogen; cyano; C1-C6 alkyloxy optionally substituted with one or more group(s) selected from cyano, halogen, hydroxy, non-aromatic carbocyclyl and aromatic heterocyclyl; C1-C6 alkyl optionally substituted with one or more group(s) selected from cyano, halogen, and hydroxy; non-aromatic carbocyclyl optionally substituted with one or more group(s) selected from cyano and halogen; non-aromatic heterocyclyl optionally substituted with one or more group(s) selected from cyano and aromatic heterocyclyl; C1-C6 alkenyloxy optionally substituted with one or more group(s) selected from cyano, halogen, and hydroxy; C1-C6 alkynyloxy optionally substituted with one or more group(s) selected from cyano, halogen, and hydroxy; or aromatic heterocyclyl optionally substituted with one or more group(s) selected from C1-C6 alkyl;
R^{7b} is a hydrogen atom, halogen, C1-C3 alky optionally substituted with halogen, or amino;
R⁸ is a hydrogen atom or halogen; and
R⁹ is halogen;
provided that when R⁸ is a hydrogen atom, then s is an integer from 1 to 3;
provided that the following compounds are excluded: 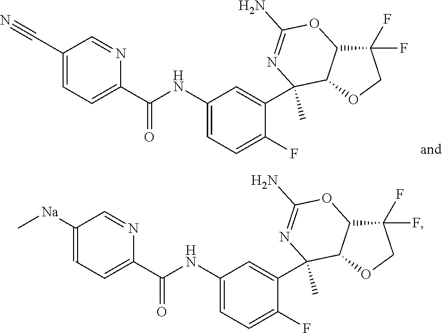
or its pharmaceutically acceptable salt.

2. The compound according to claim 1, wherein s is an integer from 1 to 3; and R³ is each independently C1-C8 alkyl optionally substituted with one or more group(s) selected from halogen, cyano, C1-C3 alkyloxy, C1-C3 haloalkyloxy, 3- to 6-membered non-aromatic carbocyclyl optionally substituted with halogen, and 3- to 6-membered non-aromatic heterocyclyl optionally substituted with halogen,
or its pharmaceutically acceptable salt.

3. The compound according to claim 1, wherein
A₁ is CR⁶ or N; A₂ is CR¹⁰ or N;
provided that when A₁ is N, then A₂ is CR¹⁰,
or its pharmaceutically acceptable salt.

4. The compound according to claim 1, wherein A₁ is CR⁶; A₂ is CR¹⁰; R⁶ is fluoro or chloro; and R¹⁰ is a hydrogen atom,
or its pharmaceutically acceptable salt.

5. The compound according to claim 1,
wherein R⁵ is a hydrogen atom or fluoro,
or its pharmaceutically acceptable salt.

6. The compound according to claim 1,
wherein R⁵ is a hydrogen atom,
or its pharmaceutically acceptable salt.

7. The compound according to claim 1, wherein 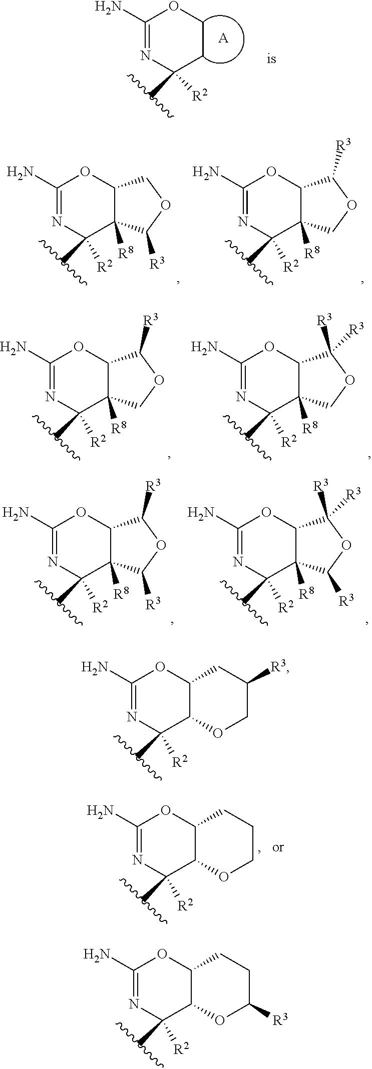
or its pharmaceutically acceptable salt.

8. The compound according to claim 7, wherein 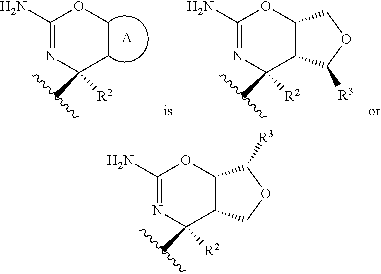
or its pharmaceutically acceptable salt.

9. The compound according to claim 1, wherein 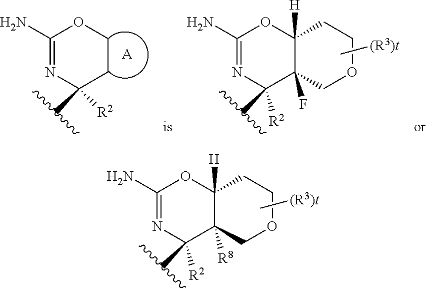
or its pharmaceutically acceptable salt.

10. The compound according to claim 1, wherein R² is monofluoromethyl or difluoromethyl,
or its pharmaceutically acceptable salt.

11. The compound according to claim 1, wherein R³ is each independently C1-C6 alkyl optionally substituted with one or more substituents selected from the group consisting of halogen, cyano, and alkyloxy,
or its pharmaceutically acceptable salt.

12. The compound according to claim 11,
wherein R³ is each independently methyl or C1-C6 alkyl substituted with fluoro,
or its pharmaceutically acceptable salt.

13. The compound according to claim 1, wherein 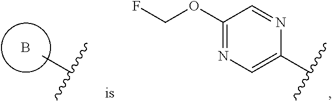
or its pharmaceutically acceptable salt.

14. The compound according to claim 1, selected from the group consisting of Compound I-001, I-002, I-003, I-004, I-005, I-006, I-013, I-014, I-015, I-030, I-082, I-112, I-116, and I-117,
or its pharmaceutically acceptable salt.

15. A pharmaceutical composition comprising the compound according to claim 1, or its pharmaceutically acceptable salt.

16. A BACE 1 inhibitor comprising the compound according to claim 1, or its pharmaceutically acceptable salt.

17. (canceled)

18. The pharmaceutical composition according to claim 15 that is effective for treating or preventing Alzheimer dementia, mild cognitive impairment or prodromal Alzheimer's disease, for preventing the progression of Alzheimer dementia, mild cognitive impairment, or prodromal Alzheimer's disease, or for preventing the progression in a patient asymptomatic at risk for Alzheimer dementia.

19. (canceled)

20. A method for inhibiting BACE1 activity comprising administering the compound according to claim 1, or its pharmaceutically acceptable salt.

21. A method for treating or preventing Alzheimer dementia, mild cognitive impairment or prodromal Alzheimer's disease, for preventing the progression of Alzheimer dementia, mild cognitive impairment, or prodromal Alzheimer's disease, or for preventing the progression in a patient asymptomatic at risk for Alzheimer dementia, comprising: administering the compound according to claim 1, or its pharmaceutically acceptable salt.
